SEQUENCE LISTING (56 sequences)

SEQ ID NO: 1 - protein, 1443 aa, synthetic construct
MDYKDDDDKPKKKRKVTGSRALSGGGSGGSGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKGGSGGSGGSGGSGGSVVSASALSPSLIN

SEQ ID NO: 2 - protein, 2050 aa, synthetic construct
MDYKDDDDKPKKKRKVTGSRALSGGGSGGSGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKGGSGGSGGSGGSGGSVVSIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 3 - protein, 2050 aa, synthetic construct
MDYKDDDDKPKKKRKVTGSRALSGGGSGGSGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKGGSGGSGGSGGSGGSVVSIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYGSYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 4 - protein, 2050 aa, synthetic construct; identical to SEQ ID NO: 2

SEQ ID NO: 5 - protein, 2050 aa, synthetic construct
MDYKDDDDKPKKKRKVTGSRALSGGGSGGSGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKGGSGGSGGSGGSGGSVVSIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLYSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 6 - protein, 830 aa, synthetic construct
MSRLDKSKIINGALELLNGVGIEGLTTRKLAQKLGVEQPTLYWHVKNKRALLDALPIEMLDRHHTHSCPLEPESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGGDYKDHDGDYKDHDIDYKDDDDKGGSGSGSIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEGDGSSVSYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDGSGEGRGSLL

SEQ ID NO: 7 - protein, 1180 aa, synthetic construct
MGPKKKRKVGSGSAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERPAYPYDVPDYAGGSGGSGGSVVSIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 8 - protein, 1987 aa, synthetic construct
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLANLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNKRPAATKKAGQAKKKKGGSGGSGGSGGSGGSVVSIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHENLIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDGSYPYDVPDYAYPYDVPDYAYPYDVPDYA

SEQ ID NO: 9 - protein, 620 aa, synthetic construct
VVSIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 10 - protein, 7 aa, synthetic construct
PKKKRKV

SEQ ID NO: 11 - protein, 16 aa, synthetic construct
KRPAATKKAGQAKKKK

SEQ ID NO: 12 - protein, 15 aa, synthetic construct
GGSGGSGGSGGSGGS

SEQ ID NO: 13 - protein, 10 aa, synthetic construct
ASALSPSLIN

SEQ ID NO: 14 - RNA, 20 nt, synthetic construct
  source 1..20: mol_type = other RNA
acttcaggttcaaagaagcc

SEQ ID NO: 15 - RNA, 20 nt, synthetic construct
  source 1..20: mol_type = other RNA
ccctgggtggggaaaaccag

SEQ ID NO: 16 - RNA, 20 nt, synthetic construct
  source 1..20: mol_type = other RNA
ttttccccacccagggccta

SEQ ID NO: 17 - RNA, 20 nt, synthetic construct
  source 1..20: mol_type = other RNA
agggagaacggggcctaccg

SEQ ID NO: 18 - RNA, 20 nt, synthetic construct
  source 1..20: mol_type = other RNA
cagacatctaataccacggt

SEQ ID NO: 19 - RNA, 20 nt, synthetic construct
  source 1..20: mol_type = other RNA
agtgataagacacccgcttt

SEQ ID NO: 20 - RNA, 20 nt, synthetic construct
  source 1..20: mol_type = other RNA
gcatgacaaaggtgccgtga

SEQ ID NO: 21 - RNA, 20 nt, synthetic construct
  source 1..20: mol_type = other RNA
gtgccgtgatggttctgtcc

SEQ ID NO: 22 - RNA, 20 nt, synthetic construct
  source 1..20: mol_type = other RNA
ggaggaacatgcttcggaac

SEQ ID NO: 23 - RNA, 20 nt, synthetic construct
  source 1..20: mol_type = other RNA
cctgccttttgggcagttaa

SEQ ID NO: 24 - RNA, 20 nt, synthetic construct
  source 1..20: mol_type = other RNA
tcggcctttaactgcccaaa

SEQ ID NO: 25 - RNA, 20 nt, synthetic construct
  source 1..20: mol_type = other RNA
ggtctgccggaaggtctaca

SEQ ID NO: 26 - RNA, 19 nt, synthetic construct
  source 1..19: mol_type = other RNA
tgtactctctgaggtgctc

SEQ ID NO: 27 - RNA, 19 nt, synthetic construct
  source 1..19: mol_type = other RNA
acgcagataagaaccagtt

SEQ ID NO: 28 - RNA, 19 nt, synthetic construct
  source 1..19: mol_type = other RNA
catcaagtcagccatcagc

SEQ ID NO: 29 - RNA, 19 nt, synthetic construct
  source 1..19: mol_type = other RNA
gagtcaccctcctggaaac

SEQ ID NO: 30 - RNA, 19 nt, synthetic construct
  source 1..19: mol_type = other RNA
gctagggatgaagaataaa

SEQ ID NO: 31 - RNA, 19 nt, synthetic construct
  source 1..19: mol_type = other RNA
ttgaccaatagccttgaca

SEQ ID NO: 32 - RNA, 19 nt, synthetic construct
  source 1..19: mol_type = other RNA
tgcaaatatctgtctgaaa

SEQ ID NO: 33 - RNA, 19 nt, synthetic construct
  source 1..19: mol_type = other RNA
aaattagcagtatcctctt

SEQ ID NO: 34 - RNA, 20 nt, synthetic construct
  source 1..20: mol_type = other RNA
aatatgtcacattctgtctc

SEQ ID NO: 35 - RNA, 20 nt, synthetic construct
  source 1..20: mol_type = other RNA
ggactatgggaggtcactaa

SEQ ID NO: 36 - RNA, 20 nt, synthetic construct
  source 1..20: mol_type = other RNA
gaaggttacacagaaccaga

SEQ ID NO: 37 - RNA, 20 nt, synthetic construct
  source 1..20: mol_type = other RNA
gccctgtaagcatcctgctg

SEQ ID NO: 38 - RNA, 20 nt, synthetic construct
  source 1..20: mol_type = other RNA
gatttctaagaaaccagttg

SEQ ID NO: 39 - RNA, 20 nt, synthetic construct
  source 1..20: mol_type = other RNA
ttctggagctggagcaaagc

SEQ ID NO: 40 - RNA, 20 nt, synthetic construct
  source 1..20: mol_type = other RNA
gatttctaagaaaccagttg

SEQ ID NO: 41 - RNA, 20 nt, synthetic construct
  source 1..20: mol_type = other RNA
cttgacctggaagactcagc

SEQ ID NO: 42 - RNA, 20 nt, synthetic construct
  source 1..20: mol_type = other RNA
ctgagaacgacccctaggag

SEQ ID NO: 43 - RNA, 20 nt, synthetic construct
  source 1..20: mol_type = other RNA
gaggaatgtgcaaacggccc

SEQ ID NO: 44 - RNA, 20 nt, synthetic construct
  source 1..20: mol_type = other RNA
agcctacaccctggaggtac

SEQ ID NO: 45 - RNA, 20 nt, synthetic construct
  source 1..20: mol_type = other RNA
ccactggggatagttccctg

SEQ ID NO: 46 - RNA, 20 nt, synthetic construct
  source 1..20: mol_type = other RNA
tgacaccgtgaactttccca

SEQ ID NO: 47 - RNA, 20 nt, synthetic construct
  source 1..20: mol_type = other RNA
taagttggtgtagacgcctg

SEQ ID NO: 48 - RNA, 20 nt, synthetic construct
  source 1..20: mol_type = other RNA
tgaattctaggccaagactt

SEQ ID NO: 49 - RNA, 20 nt, synthetic construct
  source 1..20: mol_type = other RNA
ccttggtgaagtctcctttg

SEQ ID NO: 50 - RNA, 20 nt, synthetic construct
  source 1..20: mol_type = other RNA
ggtggggcatctttatagct

SEQ ID NO: 51 - RNA, 20 nt, synthetic construct
  source 1..20: mol_type = other RNA
acattacacacaaataggga

SEQ ID NO: 52 - RNA, 20 nt, synthetic construct
  source 1..20: mol_type = other RNA
gttgagggttttttggtggg

SEQ ID NO: 53 - RNA, 20 nt, synthetic construct
  source 1..20: mol_type = other RNA
gtcccctccaccccacagtg

SEQ ID NO: 54 - RNA, 20 nt, synthetic construct
  source 1..20: mol_type = other RNA
gtattactgatattggtggg

SEQ ID NO: 55 - RNA, 20 nt, synthetic construct
  source 1..20: mol_type = other RNA
catggtgatacatttgcaaa

SEQ ID NO: 56 - RNA, 23 nt, synthetic construct
  source 1..23: mol_type = other RNA
cattgagatagtgtggggaaggg

(19) United States
(12) Patent Application Publication
(10) Pub. No.: US 2026/0226429 A1
(43) Pub. Date: Aug. 6, 2026

(54) ENGINEERED LYSINE ACYLTRANSFERASES AND METHODS OF USE THEREOF

(71) Applicant: William Marsh Rice University, Houston, TX (US)

(72) Inventors: Isaac HILTON, Houston, TX (US); Jacob GOELL, Houston, TX (US); Shriya SHAH, Houston, TX (US); Sunghwan KIM, Houston, TX (US)

(73) Assignee: William Marsh Rice University, Houston, TX (US)

(21) Appl. No.: 19/155,146

(22) PCT Filed: Feb. 8, 2024
(86) PCT No.: PCT/US2024/015007

§ 371 Date: Aug. 8, 2025

Related U.S. Application Data
(60) Provisional application No. 63/484,142, filed on Feb. 9, 2023.

(51) Int. Cl. C12N 9/10; C12N 9/22; C12N 15/11; C12N 15/90

(52) U.S. Cl. CPC C12N 9/1029; C12N 9/226; C12N 15/11; C12N 15/907; C12Y 203/01048; C07K 2319/00; C12N 2310/20

(57) ABSTRACT

The present disclosure provides engineered lysine acyltransferases, such as acetyltransferases (e.g., p300 or CBP), comprising one or more mutations which decrease cellular toxicity as compared to endogenous lysine acyltransferase, such as acetyltransferase (e.g., p300 or CBP). Further provided are methods for the use of the engineered lysine acyltransferase, such as acetyltransferase (e.g., p300 or CBP), for activating gene expression and cell therapy.

### REFERENCE TO RELATED APPLICATIONS

The present application claims the priority benefit of United States provisional application No. 63/484,142, filed Feb. 9, 2023, the entire contents of which is incorporated herein by reference.

### STATEMENT REGARDING FEDERALLY SPONSORED RESEARCH

The invention was made with government support under Grant Nos. R35GM143532 and R561HG012206 awarded by the National Institutes of Health. The government has certain rights in the invention.

### SEQUENCE LISTING

This application contains a Sequence Listing XML, which has been submitted electronically and is hereby incorporated by reference in its entirety. Said XML Sequence Listing, created on Feb. 8, 2024, is named RICEP0121WO.xml and is 64,295 bytes in size.

### BACKGROUND

### 1. Field

The disclosure relates generally to the field of molecular biology. More particularly, it concerns an engineered lysine acyltransferase, such as acetyltransferases and methods of use thereof for target gene activation.

### 2. Related Art

Epigenome editing in human cells requires a DNA-binding domain fused to an epigenome-modifying domain that elicits a functional effect on gene regulatory outputs and the chromatin environment. While substantial success has been derived in engineering transactivation domains that recruit RNA Pol II to modulate transcription, adapting lysine-modifying enzymes has proven to be much more difficult. Given their propensity to modify proteins in a DNA-binding domain independent manner, these proteins, especially those that harbor histone-modifying transcriptional activation potential, often have highly promiscuous activity that can render them toxic to the cell.

Over the past decade, catalytically deactivated CRISPR-Cas (dCas) systems have emerged as powerful technologies to study epigenetic regulatory mechanisms in eukaryotic cells (Goell & Hilton 2021, Nakamura et al., 2021). Epigenome editing platforms leverage the dCas protein as a scaffold to recruit epigenetic modifiers, or effectors, to targeted loci of interest (Goell & Hilton 2021, Nakamura et al., 2021). This capability has been instrumental for understanding the fundamental principles of heritable gene silencing (Nuñez et al., 2021), enhancer biology (Li et al., 2020; Klann et al., 2017; Fulco et al., 2016), and transcriptional activation/repression (O'Geen et al., 2017; Matharu et al., 2019). By site-specifically depositing specific epigenetic modification(s) at the locus of interest, epigenome editing enables interrogation of whether the addition or erasure of a specific modification is necessary or instructive for the transcription of endogenous loci. The programmable nature of this class of technologies makes them flexible and highly useful for elucidating causal relationships between epigenetic modifications and genomic activity. Despite the potential of such tools, a better understanding of the cytotoxicity and off-target profiles these effector domains harbor is necessary for greater adoption and future clinical applications. Epigenome editing effector domains with active enzymatic activity are especially susceptible to pervasive guide RNA independent off-targets and may require substantial engineering to overcome these concerns (Nuñez et al., 2021; Hofacker et al., 2020; Pflueger et al., 2018; Gemberling et al., 2021).

Lysine acylation is a post-translational modification (PTM) that plays a key role in regulating chromatin structure and gene expression (Ali et al., 2018). In particular, acylation of lysine residues on histone proteins has been shown to play a crucial role in determining chromatin accessibility and recruitment of epigenetic reader domains in eukaryotes (Nitsch et al., 2021; Dai et al., 2021). Lysine acylation is dynamic and is regulated by a number of enzymes, allowing for the regulation of gene expression in response to various stimuli (Fellows et al., 2018; Trefely et al., 2020; Sabari et al., 2015; Sheikh & Akhtar, 2019; Shvedunova & Akhtar, 2022). It has also been shown that many different lysine acetyltransferases harbor the ability to deposit longer-chain acylations onto their substrates in addition to acetylation (Tan et al., 2011). One such enzyme is p300, and its paralog CBP, which have been shown to deposit propionyl, butyryl, crotonyl, lactyl, and other acylations onto histones (Kaczmarska et al., 2017). These acylations are largely derived through metabolic processes such as fatty acid oxidation and the tricarboxylic acid (TCA) cycle, where they are generated as an adduct to coenzyme A and used as a substrate by lysine acyltransferases and deposited onto histones and other proteins (Dai et al., 2021). As a result, these acylations are highly context-specific and are present in cell types, wherein specific metabolic pathways are active and corresponding feedstocks are available. Longer-chain acylations are chemically distinct from acetylation. However, they are thought to serve similar functions and have been linked to active transcription despite having different epigenetic reader affinities (Flynn et al., 2015; Filippakopoulos et al., 2012; Li et al., 2016). Despite this fundamental importance, there are currently no tools with which to study the roles of different acylations at endogenous loci in living cells. Thus, there is an unmet need for engineered lysine-modifying enzymes.

### SUMMARY

In a first embodiment, the present disclosure provides a polypeptide comprising an engineered human lysine acyltransferase (e.g., lysine acetyltransferase (KAT) or lysine crotonyltransferase) or effector domain thereof comprising at least one amino acid mutation in the acyl-CoA binding site.

In some aspects, the human lysine acyltransferase (e.g., KAT) is p300 or CBP. In certain aspects, the at least one amino acid mutation is between amino acids 1380-1420. In some aspects, the at least one mutation is an I1417N, I1395G, P1388C, Y1397Q, Y1397A, and/or C1385Q mutation. In some aspects, the at least one mutation is an Q1390R, K1407Y. L1409K, and/or I1417N mutation. In certain aspects, the p300 comprises an I1417N mutation. In particular aspects, the p300 comprising a I1417N mutation has decreased cellular toxicity as compared to endogenous p300.

In some aspects, the lysine acyltransferase (e.g., KAT) is fused to a reverse Tetracycline repressor (TetR or rTetR) protein. In certain aspects, the lysine acyltransferase (e.g., KAT) is fused to a (Clustered Regularly Interspaced Short Palindromic Repeats associated) Cas protein. In particular aspects, the Cas protein is dCas9 or ddCas12a. In certain aspects, the Cas protein is dCasMINI, dCas12i, dCas12F, or dCas12j. In specific aspects, the lysine acyltransferase (e.g., KAT) is fused to a Zinc finger or TALE protein.

In some aspects, the polypeptide further comprises a reporter. In certain aspects, the reporter is a fluorescent protein, such as mCherry.

In some aspects, the engineered acyltransferase may comprise a sequence having at least 80%, 85%, 90%, 91%, 92%, 93%, 94%, 95%, 96%, 97%, 98%, 99%, or 100% sequence similarity to SEQ ID Nos: 1-8. The acyltransferase may comprise p300 having at least 80%, 85%, 90%, 91%, 92%, 93%, 94%, 95%, 96%, 97%, 98%, 99%, or 100% sequence similarity to SEQ ID NO: 9 and/or 1, 2, 3, 4, or 5 point mutations to SEQ ID NO: 9. The acyltransferase may comprise a nuclear localization sequence having at least 80%, 85%, 90%, 91%, 92%, 93%, 94%, 95%, 96%, 97%, 98%, 99%, or 100% sequence similarity to SEQ ID NO: 10 (PKKKRKV) or SEQ ID NO: 11 (KRPAATKKAGQAKKKK). The acyltransferase may comprise a glycine-serine linker having at least 80%, 85%, 90%, 91%, 92%, 93%, 94%, 95%, 96%, 97%, 98%, 99%, or 100% sequence similarity to SEQ ID NO: 12 (GGSGGSGGSGGSGGS) and/or an entry site having at least 80%, 85%, 90%, 91%, 92%, 93%, 94%, 95%, 96%, 97%, 98%, 99%, or 100% sequence similarity to SEQ ID NO: 13 (ASALSPSLIN).

A further embodiment provides a fusion protein comprising the engineered lysine acyltransferase (e.g., lysine acetyltransferase (KAT) or lysine crotonyltransferase) polypeptide of the present embodiments or aspects thereof (e.g., an engineered human lysine acyltransferase (e.g., lysine acetyltransferase (KAT) or lysine crotonyltransferase) or effector domain thereof comprising at least one amino acid mutation in the acyl-CoA binding site) and a Cas polypeptide, Zinc finger polypeptide, or TALE polypeptide. In some aspects, the Cas polypeptide is dCas9, ddCas12a, dCasMINI, dCas12i, dCas12f, or dCas12j. In certain aspects, the fusion protein further comprises a linker between the engineered lysine acyltransferase (e.g., lysine acetyltransferase (KAT) or lysine crotonyltransferase) and the Cas polypeptide, Zinc finger polypeptide, or TALE polypeptide. In certain aspects, the fusion protein activates transcription of a target gene by activating distal regulatory elements.

Further provided herein is an isolated polynucleotide encoding the fusion protein of the present embodiments or aspects thereof (e.g., engineered lysine acyltransferase (e.g., lysine acetyltransferase (KAT) or lysine crotonyltransferase) or effector domain thereof comprising at least one amino acid mutation in the acyl-CoA binding site fused to a Cas polypeptide. Zinc finger polypeptide, or TALE polypeptide). Another embodiment provides a vector comprising the isolated polynucleotide encoding the fusion protein of the present embodiments or aspects thereof (e.g., engineered lysine acyltransferase (e.g., lysine acetyltransferase (KAT) or lysine crotonyltransferase) or effector domain thereof comprising at least one amino acid mutation in the acyl-CoA binding site fused to a Cas polypeptide, Zinc finger polypeptide, or TALE polypeptide). In some embodiments, the isolated polynucleotide encoding the fusion protein of the present embodiments or aspects thereof is delivered as mRNA. In some aspects, the vector is a viral vector. In certain aspects, viral vector is a lentiviral vector, an adenoviral vector, a retroviral vector, a vaccinia viral vector, an adeno-associated viral vector, a herpes viral vector, or a polyoma viral vector. In some aspects, the viral vector is a lentiviral vector. In certain aspects, the lentiviral vector has increased packaging and transduction efficiency as compared to a lentiviral vector encoding endogenous p30t.

A further embodiment provides a DNA targeting system comprising the fusion protein of the present embodiments or aspects thereof (e.g., engineered lysine acyltransferase (e.g., lysine acetyltransferase (KAT) or lysine crotonyltransferase) or effector domain thereof comprising at least one amino acid mutation in the acyl-CoA binding site fused to a Cas polypeptide, Zinc finger polypeptide, or TALE polypeptide) and at least one guide RNA (gRNA).

In some aspects, the at least one gRNA targets a target region, the target region comprises a target enhancer, target regulatory element, a cis-regulatory region of a target gene, or a trans-regulatory region of a target gene. In certain aspects, the target region is a distal or proximal cis-regulatory region of the target gene. In some aspects, the target region is an enhancer region or a promoter region of the target gene. In some aspects, the target gene is an endogenous gene or a transgene. In particular aspects, the DNA targeting system comprises between one and ten different gRNAs. In some aspects, the DNA targeting system comprises one gRNA. In some aspects, the target region is located on the same chromosome as the target gene. In specific aspects, the target region is located about 1 base pair to about 100,000 or about 1,000,000 base pairs upstream of a transcription start site of the target gene. In some aspects, the target region is located about 1004) base pairs to about 50,000 base pairs upstream of the transcription start site of the target gene. In certain aspects, the target region is located on a different chromosome as the target gene. In some aspects, the target region is at least one of HS2 enhancer of the human β-globin locus, distal regulatory region (DRR) of the MYOD gene, core enhancer (CE) of the MYOD gene, proximal (PE) enhancer region of the OCT4 gene, or distal (DE) enhancer region of the OCT4 gene. In some aspects, the target gene is low-density lipoprotein receptor (LDLR) gene, PCSK9, ATP7A, ATP7B, or a gene in Tables 1-3. In certain aspects, the target region is the promoter of ANO5, IRS1, SCN2A, or EIF1AX. In some aspects, the target region is the enhancer of PCTP, LINC01993, LGALS3BP, ZNF180, THUMPD2, LINC01033, SNX18, HIST1H4H, HIST1H2BO, or SLC35B2.

Another embodiment provides a method for activating gene expression of a target gene in a cell comprising contacting the cell with the polypeptide of the present embodiments or aspects thereof (e.g., engineered lysine acyltransferase (e.g., lysine acetyltransferase (KAT) or lysine crotonyltransferase) or effector domain thereof comprising at least one amino acid mutation in the acyl-CoA binding site), the fusion protein of the present embodiments or aspects thereof (e.g., engineered lysine acyltransferase (e.g., lysine acetyltransferase (KAT) or lysine crotonyltransferase) or effector domain thereof comprising at least one amino acid mutation in the acyl-CoA binding site fused to a Cas polypeptide. Zinc finger polypeptide, or TALE polypeptide), a vector of the present embodiments or aspects thereof, or a DNA targeting system of the present embodiments or aspects thereof. In some aspects, the cell is contacted with a polypeptide of the present embodiments or aspects thereof and a DNA-binding domain for the target gene. In some aspects, the cell is contacted with a fusion protein, a vector, or a DNA targeting system of the present embodiments or aspects thereof and at least one gRNA.

A further embodiment provides a method for cell therapy comprising activating a target gene by introducing the engineered lysine acyltransferase (e.g., lysine acetyltransferase (KAT) or lysine crotonyltransferase) of present embodiments or aspects thereof and a DNA-binding domain for said target gene. In some aspects, the method is ex vivo. In some aspects, the method is in vivo. In certain aspects, the disease comprises haploinsufficiency. In some aspects, the disease is nonalcoholic fatty liver disease (NASH), sickle cell disease, alpha thalassemia, or beta thalassemia. In certain aspects, the method reduces T cell exhaustion. In some aspects, the disease is chronic liver disease. The target gene for liver disease may be LDLR or a gene selected from Tables 1-3. In some aspects, the disease is Wilson's disease or a central nervous system (CNS) disease, such as but not limited to Dravet syndrome. Rett syndrome or Amyotrophic lateral sclerosis (ALS).

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Enzymatic activity of various p300 mutants by SV40 reporter targeting shown as percentage of Cit+ cells.

FIG. 2: Relative H3K27ac levels of various p300 mutants targeted to HS2 enhancer.

FIG. 3: Select mutant variants from high throughput screen exhibit higher stability and activity than wild-type p300 in arrayed screen validation.

FIG. 4: Mutants from high throughput screen show varied levels of enhancer activation, gRNA targeting HS2 enhancer qPCR measurement at HBG1.

FIG. 5: CUT&RUN profiling of H3K27ac in HEK293T cells reveal reduced off-targets in p300 I1417N transfected cells.

FIGS. 6A-6F: Targeting of p300 variants to OCT4 regulatory elements results in divergent transcriptional responses. (FIG. 6A) Construct designs for testing dCas9-p300 variants. (FIG. 6B) Western blot analysis of whole proteome lysine crotonylation probed using anti-KCr antibody in HEK293T cells overexpressing dCas9-p300 variant. (FIG. 6C) In vitro acylation activity assay of dCas9-p300 variants measured using the acyl-CoA co-factor indicated on the X and Y axes. Activity was measured using the N-terminal H3 peptide (n=5, mean±sem). (FIG. 6D) Top: Schematic of the OCT4 locus with small lower rectangles indicating target gRNA location relative to transcription start site. Bottom: RT-qPCR of OCT4 gene expression upon dCas9-p300 variant targeting in HEK293T cells (n=3, mean±sem). (FIG. 6E) Relative enrichment of indicated histone modification measured by CUT&RUN-qPCR following delivery of dCas9-p300 variant and OCT4 distal enhancer targeting gRNAs in HEK293T cells (n=4-6, mean±sem). (FIG. 6F) Same as (E) with OCT4 promoter targeting gRNAs (n=4-6, mean±sem).

FIGS. 7A-7F: Developing a high throughput screen to assess protein expression and transcriptional activity in mammalian cells. (FIG. 7A) Schematic of the screening pipeline. A reporter gene is stably integrated into the AAVS1 locus with 5× upstream TetO sites in HEK293T cells. A library of p300 variants fused to rTetR is transduced into the reporter cell line, selected with blasticidin, and induced with doxycycline for 48 hours before sorting and sequencing. (FIG. 7B) PDB (5LKU) crystal structure of the p300 core domain bound to Coenzyme A. (FIG. 7C) Citrine and mCherry fluorescence levels of an individual denoted variant of rTetr-p300 were measured by flow cytometry 11 days after transduction. (FIG. 7D) Heatmap illustrating effects of all single mutations on citrine activation reporter. Ratios are shifted to the median of negative degenerate sequence controls (n=2). (FIG. 7E) Heatmap illustrating effects of all single mutations on mCherry expression reporter. Ratios are shifted to the median of negative degenerate sequence controls (n=2). (FIG. 7F) Top: Construct design for testing epigenome effector domains protein stability against mRNA expression levels. Bottom: Representative flow cytometry plots of mRNA expression (mChe) across 4 different epigenome editor effector domains with and without catalytic activity.

FIGS. 8A-8I: I1417N mutation reduces p300 cytotoxicity and improves viral delivery while preserving activity. (FIG. 8A) Scatter plot depicting the relationship between reporter activation (x-axis) and protein expression (y-axis). Shaded are the top 100/bottom variants summing the log ratios of both fluorescent readouts. Hits from shaded regions were selected for individual validation. Data showing that mutations flanking the acyl-CoA binding site (across the entirety of the scanned region 1381-1420) but outside of the 1398-1399 residues that are known catalytic mutants can be engineered to modulate expression and activity of the enzyme. Notably, mutations that occur in the beta strands composing residues 1383-1385 and 1392-1400 along with the linker region between these are enriched for both highly expressed and active variants. Similarly, there are hits occurring in the helices formed by residues 1407-1409 and 1410-1428. These regions are purported to govern the conformation of the acetyl-CoA binding pocket and may govern the recognition of protein substrates. By changing the interactions with either the acyl-CoA molecule in the acyl portion or the CoA portion, proteins were selectively enriched for certain acyl substrates or globally effecting recognition by altering CoA binding, respectively. This aspect is what drives the expression and activity of the enzyme. Notably, hydrophobic (L, Y, I) and structurally distinct residues (P, C) were enriched as hits suggesting that these play important roles in the assembly of the acyl-CoA binding module. (FIG. 8B) % of mCherry-positive cells across each single construct validation in transient transfection measured by flow cytometry. (FIG. 8C) RT-qPCR of HBG1 gene expression upon dCas9-p300 variant targeting to HBG1 (x-axis) and HS2 enhancer (y-axis) (n=3, mean±sem). (FIG. 8D) RT-qPCR of OCT4 gene expression upon dCas9-p300 variant targeting across OCT4 distal enhancer, proximal enhancer, and promoter, respectively (n=3, mean f sem). (FIG. 8E) Western blot analysis of HEK293T cells overexpressing dCas9-p300 variant probing for anti-Cas9 with anti-Tubulin as loading control. (FIG. 8F) Fluorescence microscopy image of HEK293T cells transiently transfected with dCas9-p300 variant bicistronically expressed with mCherry and scrambled gRNA. Top panel: mCherry channel fluorescence. Bottom panels. DAPI staining. Scale bar: 100 um. (FIG. 8G) Cell viability as measured using ALAMARBLUE™ cell viability reagent 48 hours post-transient transfection with dCas9-p300 variant and scrambled gRNA in HEK293T cells. (FIG. 8H) % of mCherry-positive cells in 2% v/v transduced K562 cells measured by flow cytometry. (FIG. 8I) RT-qPCR of HBG1 gene expression upon dCas9-p300 or CBP variant targeting to HBG1 (x-axis) and HS2 enhancer (y-axis) (n=3, mean±sem).

FIGS. 9A-9E: Immunoprecipitation-mass spectrometry (IP-MS) reveals different classes of protein interactions within different p300 variants. (FIG. 9A) Venn diagram of hits called in IP-MS experiment (n=2, P<0.05). (FIG. 9B) Dot chart showing top 84 protein hits by greatest −log(P-value) relative to dCas9 only bait. Classes of interactions are hierarchically clustered and subgroups are named on characteristic similarity. (FIG. 9C) Volcano plot of the protein interactome of the p300 I1417N variant relative to the wild-type. (FIG. 9D) Gene ontology analysis of 1417N-enriched hits. (FIG. 9E) Percentage BFP+ cells in GFP-BFP conversion assay via prime editing co-transfected with dCas9-p300 variant targeting the same site in HEK293T-eGFP-PEST cells (n=3, mean±sem).

FIGS. 10A-10F: dCas9-p30011417N has reduced off-targeting activity. (FIG. 10A) Transcriptomes of HEK293T cells transiently expressing dCas9-p300 WT compared against dCas9 (n=2 biological replicates). Each black dot indicates the expression level of an individual gene. Dashed lines indicate the 2× difference between sample groups. (FIG. 10B) Genomic coordinates spanning ~5,248,000 to ~5,255,256 bp of human chromosome 11 (GRCh38/hg38) are shown along with H2BK20ac, H3K27ac, H3K4me3, and H3K27me3 CUT&RUN enrichment data with the indicated dCas9-p300 variant and 4 HBG1-targeting sgRNAs in HEK293T cells (n=2 biological replicates). (FIG. 10C) Profile of H3K27ac enrichment at transcription start sites. (FIGS. 10D-10F) Relative enrichment of indicated histone modification (H2BK20ac, H3K4me3, or H3K27me3) measured by CUT&RUN-qPCR following delivery of dCas9-p300 variant and HBG1 promoter targeting gRNAs in HEK293T cells (n=4, mean±sem).

FIGS. 11A-11F: A high MOI Perturb-seq screen to identify effector-specific cis regulatory elements. (FIG. 11A) Schematic of the experimental framework. Monoclonal KS62 cells expressing dCas9-p300 fusions were transduced at high MOI (~20) to introduce sgRNAs targeting regulatory elements of interest followed by scRNA-seq. (FIG. 11B) Violin plots of expression change over the null control of all statistically significant hits across p300 WT and p300 I1417N (n=48 and 24 hits, respectively). (FIGS. 11C-11D) Volcano plot shows the statistically significant hits of each effector. An FDR of 0.10 w as used after a Benjamini-Hochberg correction was used to call hits. (FIGS. 11E-11F) Violin plots of normalized expression levels compared to the null control of select genes across p300 WT and p300 I1417N targeting promoters/enhancers.

FIGS. 12A-12E: Targeting of p300 variants to OCT4 regulatory elements results in divergent transcriptional responses, related to FIG. 12. (FIG. 12A) Jaccard similarity scores of ChIP-seq data indicating the correlation of specific histone modifications and transcription factors at specific sites in HCT116 cells. Data was accessed via GSE96035 for H3K18cr and ENCBS847SOB for the rest of the groups. (FIG. 12B) Western blot analysis of construct expression probed using an anti-Cas9 antibody with an anti-Tubulin loading control in HEK293T cells transfected with different amounts of dCas9-p300 variant plasmid DNA. (FIG. 12C) RT-qPCR of promoter gene expression upon dCas9-p300 variant targeting in HEK293T cells (n=4-6, mean±sem). (FIG. 12D) RT-qPCR of OCT4 gene expression upon dCas9-p300 variant targeting in HeLa cells (n=4-6, mean±sem). (FIG. 12E) RT-qPCR of enhancer gene expression upon dCas9-p300 variant targeting in HEK293T cells (n=3-6, mean±sem).

FIGS. 13A-13E: Developing a high throughput screen to assess protein expression and transcriptional activity in mammalian cells, related to FIG. 13. (FIG. 13A) Top: Construct design for testing rTetr-p300 variant activity/expression levels in transient transfection experiments. Bottom: Percentage of mCherry+ cells in transient transfection experiments in HEK293T reporter cells without doxycycline addition (n=3, mean±sem). (FIG. 13B) Top: Construct design for testing mChe-dCas9-p300 variant expression levels in transient transfection experiments. Bottom: Percentage of mCherry+ cells in transient transfection experiments in HEK293T cells (n=3, mean±sem). (FIG. 13C) Top: Construct design for testing rTetr-p300 variant activity levels in transient transfection experiments. Bottom: Percentage of mCherry+ cells in transient transfection validation experiments in HEK293T reporter cells 2 days after doxycycline addition (n=3, mean±sem). (FIG. 13D) Domain structure of p300 core with Catalogue of Somatic Mutations in Cancer (COSMIC) database mutation occurrence overlaid to correspond to location in p300 core (FIG. 13E) Ratios of GFP to mCherry expression (geometric mean. AU) across 4 different epigenome editor effector domains with and without catalytic activity (n=3, mean±sem).

FIGS. 14A-14H: I1417N mutation reduces p300 cytotoxicity and improves viral delivery while preserving activity, related to FIG. 14. (FIG. 14A) RT-qPCR of IL1RN gene expression upon dCas9-p300 variant targeting IL1RN promoter in HEK293T cells (n=3, mean±sem). (FIG. 14B) RT-qPCR of BACH2 gene expression upon dCas9-p300 variant targeting rs72928038 risk locus in K562 cells (n=4, mean±sem). (FIG. 14C) RT-qPCR of TTN gene expression upon dCas9-p300 variant targeting TTN promoter in primary mesenchymal stromal cells (n=3, mean±sem). (FIG. 14D) RT-qPCR of HBG1 gene expression upon dCas9-p300 variant targeting HS2 enhancer in HeLa cells (n=3, mean±sem). (FIG. 14E) RT-qPCR of IL1B gene expression upon ddCas12-p300 variant targeting IL1B promoter in HEK293T cells (n=3, mean±sem) (FIG. 14F) RT-qPCR of HBG1 gene expression upon dCasMINI-p300 variant targeting HBG1 promoter in HEK293T cells (n=3, mean±sem). (FIG. 14G) Relative physical lentivirus titration following lentivirus transduction of dCas9-p300 variant in HEK293T cells (n=3, mean±sem). RT-qPCR of HBG1 gene expression upon dCas9-p300 variant targeting HS2 enhancer in HeLa cells (n=3, mean±sem). (FIG. 14H) Sequence alignment of p300 and CBP flanking window of I to N mutation (I1417N in p300 and I1453N in CBP). The mutation site is demarcated below the sequence alignment in red.

FIGS. 15A-15D: Immunoprecipitation-mass spectrometry (IP-MS) reveals different classes of protein interactions within different p300 variants, related to FIG. 15. (FIG. 15A) Schematic representation of the experimental design for IP-MS study of dCas9-p300 variants. (FIG. 15B) Volcano plot of the protein interactome of the p300 I1395G variant (left) and p300 D1399Y variant (right) relative to p300 WT. Red highlights correspond to P<0.05 (n=2). (FIG. 15C) Venn diagram containing union set of shared hits between IP-MS experiment and STRING database. Names of individual hit genes are listed to the right of the Venn diagram. (FIG. 15D) Venn diagram containing union set of shared hits between IP-MS experiment and p300 acetylome database. Names of individual hit genes are listed to the right of the Venn diagram.

FIGS. 16A-16C: dCas9-p300 I1417N has reduced off-targeting activity, related to FIG. 16. (FIG. 16A) MA plots of HEK293T cell transcriptomes transiently expressing dCas9-p300 WT compared against dCas9 (n=2 biological replicates). Each black dot indicates the expression level of an individual gene. (FIG. 16B) Relative enrichment of H3K27ac measured by CUT&RUN-qPCR following delivery of dCas9-p300 variant and HBG1 promoter targeting gRNAs in HEK293T cells (n=4, mean±sem). (FIG. 16C) Metagene profile of H3K27ac enrichment.

FIGS. 17A-17H: A high MOI Perturb-seq screen to identify effector-specific cis regulatory elements, related to FIG. 17. (FIG. 17A) Representative flow cytometry histogram of mCherry bicistronically expressed with dCas9-p300 variant. (FIG. 17B) RT-qPCR of MYOD gene expression upon targeting MYOD promoter in monoclonal K562 cells stably expressing dCas9-p300 variant (n=3, mean±sem). (FIG. 17C) Histogram of gRNAs identified per cell for p300 WT (left) and p300 I1417N (right). (FIG. 17D) Histogram of the number of cells identified bearing a specific gRNA perturbation for p300 WT (left) and p300 I1417N (right). (FIG. 17E) Quantile-quantile plots of cis effects within 1 Mb (500 kb upstream and downstream) of each gRNA. Negative control target-response pairs were generated by randomly sampling all responses. Negative control p-values are shown in red and discovery p-values in blue. The grey band indicates confidence intervals. The horizontal dashed line indicates the multiple testing threshold and points above this line are called significant (FIG. 17F) Bar graph of the number of gRNA hits that activate its cognate promoter (Targeted) or a different gene (Alternate) for p300 WT and VP64/VPR. VP64/VPR was derived from Chardon et al., BioRxiv 2023. (FIG. 17G) Bar graph of the number of gRNA hits that activate a previously CRISPRi validated gene (CRISPRi Predicted) or a novel gRNA-enhancer pair (CRISPRa only) for p300 WT and VP64/VPR. VP64NPR was derived from Chardon et al, BioRxiv 2023. (FIG. 17H) Venn diagram of hits called between p300 WT and p300 I1417N using SCEPTRE processing pipeline.

FIG. 18: Gene activation of Ldlr in Hepa1-6 mouse hepatoma cell line. (Top) Relative expression levels of negatively regulated Pcsk9 gene cells (n=2-4, mean±sem). (Bottom) Relative gene expression levels of LAIR cells (n=2-4, mean±sem).

### DESCRIPTION OF ILLUSTRATIVE EMBODIMENTS

Histone acetylation, specifically at the H3K27 residue (termed H3K27ac) is a mark of active enhancer regions. This modification is largely deposited by the lysine acyltransferases p300 and its paralog CBP. Sequences between the two, especially in the core domain, are highly conserved. These paralogs acylate a broad range of proteins both in the nucleus and cytoplasm and are highly important in organismal development and cell-type specification. Both KATs have been adapted for epigenome editing applications, largely through the fusion of the catalytic core domain to a variety of different DNA-binding domains. They have been shown to robustly activate gene expression at promoters and enhancers with an especially high level of activation at the latter in comparison to existing CRISPR tools that rely on transcriptional activation domains. Because of its ability to acetylate many different protein targets, these domains induce marked cellular toxicity, drastically confining their use to a select subset of cancer cell lines and rendering biological insights derived from their use suspect due to their many indirect effects. Accordingly, certain embodiments described herein provide an engineered version of this enzyme with dampened toxicity and acetylation capacity, thus making it more amenable for use in a variety of different cell types.

In certain embodiments, the present disclosure provides engineered versions of the endogenous human lysine acyltransferase, such as lysine acetyltransferase (KAT), p300, that potently activates transcription to the same degree as the wild-type at both promoters and enhancers when recruited by CRISPR/dCas9-based systems. Importantly, this is done with minimal cellular toxicity. The present studies show that targeting an engineered lysine crotonyltransferase based on the native human p300 protein results in relatively weak levels of gene activation when targeted to endogenous enhancers yet retains strong activation when targeted to promoters. With the programmable lysine crotonyltransferase it was demonstrated that, in contrast to histone acetylation, histone crotonylation only weakly activates genes from endogenous human enhancers. However, both acylation reactions can drive potent transcription when targeted to promoters. Using a deep mutational scanning approach, a single point mutation (I1417N) was identified that drastically reduces cytotoxicity associated with the overexpression of p300 WT, without sacrificing the ability to deposit H3K27ac and activate transcription. Using quantitative mass spectrometry, it was also demonstrated that these individual point mutations reshape the protein's interactome and longer-chain acylation-specific interactions were identified as well as cytotoxicity-associated interactions. At the transcriptomic and epigenomic levels, these engineered acyltransferases display decreased off-targeting, a consideration of paramount importance for the adoption of epigenome editing methodologies in translational applications. Finally, the present dCas9-p300 I1417N fusion protein was used to perform single-cell CRISPR activation and benchmarked its performance against dCas9-p300 WT.

Thus, the present acyltransferase, such as p300, variants can be leveraged with improved delivery and cytotoxicity profiles to perform single-cell CRISPR activation and benchmarking against CRISPR activation tools. Using proteomics and a panel of engineered p300 variants, acylation-specific interactions were observed and the cytotoxicity of the wild-type p300 core domain was linked to altered activities among DNA repair machinery components. These programable epigenome editing tools can be used to perform functional genomic screens, multiplexed cell engineering, and, more broadly, understand the mechanistic role of lysine acylation in epigenetic processes. The present studies also showed that this activity is portable to other DNA-binding domains. This technology, and the methods and compositions used, are novel molecular tools to deposit histone acetylation to a variety of cis regulatory elements while preserving cell viability.

In further embodiments, the present engineered lysine acyltransferase, such as KAT p300, may be used for applications in which transcriptional activation of endogenous or engineered genes is required from either promoters or enhancers. Given p300's ability to activate gene expression from distal regulatory elements efficiently and more potently than other transcriptional activators in many cases, this is an important application of this technology. The present compositions may be used in gene or cell therapies where activation of endogenous genes is required. This is especially notable in diseases of haploinsufficiency in which only one allele of a gene is expressed and thus there is a lack of functional protein present. Being able to activate genes from enhancers is particularly useful for in vivo therapies. Because enhancers are highly cell-type specific, using this engineered version of engineered lysine acyltransferase, such as p300, may confer additional specificity for the tissue being targeted wherein the construct will not activate transcription from tissues in which the enhancer is not active.

Enhancer-mediated gene activation is especially important for mapping the non-coding genome which has not been done extensively with transcriptional activation technologies due to other tools being only weakly active at distal regulatory regions. This tool overcomes this obstacle and as such could be highly useful in dissecting which enhancers control which genes. Notably, the above discussed applications were previously inaccessible as current versions of CRISPRa tools either do not directly deposit H3K27ac or do so in a highly unspecific manner in the case of wild-type p300.

In some aspects, the present engineered lysine acyltransferase, such as p300, may be targeted to agene for liver disease such as LDLR. This approach has broad potential to treat a variety of diseases within the liver by upregulation of either agene directly from its promoter or indirectly from its enhancer (Tables 1-3). This specific approach enables facile tuning of gene expression levels through choice of the targeting region. It also has the capabilities of activating endogenous genes that modify progression of a disease driven by a loss of function mutation. This is especially useful when the gene that is knocked out exceeds the AAV packaging capacity, as this is one of the few long-lasting, validated approaches to delivering a gene of interest. In some aspects, multiple genes may be activated with this epigenome editing technology. By delivering multiple guides targeting different genes, pathways can be engineered and potentially improve liver function through multiple different approaches. The present system can activate liver disease-relevant loci from both promoters and enhancers to tune gene levels into a range that is physiologically normal.

TABLE 1
Partial list of genes involved in copper metabolism.

| Gene Symbol | Gene Name |
|---|---|
| ATOX1 | Antioxidant 1 Copper Chaperone |
| ATP7A | ATPase Copper Transporting Alpha |
| ATP7B | ATPase Copper Transporting Beta |
| CCS | Copper Chaperone For Superoxide Dismutase |
| COMMD1 | Copper Metabolism Domain Containing 1 |
| COX10 | Cytochrome C Oxidase Assembly Factor |
| COX17 | Cytochrome C Oxidase Copper Chaperone |
| COX6B1 | Cytochrome C Oxidase Subunit 6B1 |
| CTR1 | Copper Transporter 1 |
| CYB5R3 | Cytochrome B5 Reductase 3 |
| DBH | Dopamine Beta Hydroxylase |
| DCT | Dopachrome Tautomerase |
| SLC31A1 | Solute Carrier Family 31 Member 1 |
| SLC31A2 | Solute Carrier Family 31 Member 2 |
| SCO1 | SCO Cytochrome C Oxidase Assembly |
| | Protein 1 |
| SCO2 | SCO Cytochrome C Oxidase Assembly |
| | Protein 2 |
| LAP3 | Leucine Aminopeptidase 3 |
| MURC | Muscle Restricted Coiled-Coil Protein |
| PRNP | Prion Protein |
| SLC40A1 | Solute Carrier Family 40 Member 1 |

TABLE 2
Partial list of genes involved in bile acid metabolism.

| Gene Symbol | Gene Name |
|---|---|
| CYP7A1 | cytochrome P450 family 7 subfamily A |
| | member 1 |
| CYP8B1 | cytochrome P450 family 8 subfamily B |
| | member 1 |
| CYP27A1 | cytochrome P450 family 27 subfamily A |
| | member 1 |
| CYP39A1 | cytochrome P450 family 39 subfamily A |
| | member 1 |
| CYP3A4 | cytochrome P450 family 3 subfamily A |
| | member 4 |
| CYP3A5 | cytochrome P450 family 3 subfamily A |
| | member 5 |
| CYP2C8 | cytochrome P450 family 2 subfamily C |
| | member 8 |
| CYP2C9 | cytochrome P450 family 2 subfamily C |
| | member 9 |
| CYP2D6 | cytochrome P450 family 2 subfamily D |
| | member 6 |
| SLC10A1 | solute carrier family 10 member 1 |
| SLC10A2 | solute carrier family 10 member 2 |
| SLC27A5 | solute carrier family 27 member 5 |
| ABCB4 | ATP binding cassette subfamily |
| | B member 4 |
| ABCB11 | ATP binding cassette subfamily |
| | B member 11 |
| NR1H4 | nuclear receptor subfamily 1 group H |
| | member 4 |
| FGF19 | fibroblast growth factor 19 |
| FGFR4 | fibroblast growth factor receptor 4 |
| KLB | klotho beta |

TABLE 3
Partial list of genes involved in lipid metabolism.

| Gene Symbol | Gene Name |
|---|---|
| APOA1 | apolipoprotein A1 |
| APOA2 | apolipoprotein A2 |
| APOA4 | apolipoprotein A4 |
| APOB | apolipoprotein B |
| APOC1 | apolipoprotein C1 |
| APOC2 | apolipoprotein C2 |
| APOC3 | apolipoprotein C3 |
| APOD | apolipoprotein D |
| APOE | apolipoprotein E |
| APOF | apolipoprotein F |
| APOH | apolipoprotein H (beta-2-glycoprotein I) |
| CD36 | CD36 molecule (thrombospondin receptor) |
| CETP | cholesteryl ester transfer protein |
| CPT1A | camitine palmitoyltransferase 1A (liver) |
| FABP1 | fatty acid binding protein 1 |
| FABP2 | fatty acid binding protein 2 |
| FABP3 | fatty acid binding protein 3 |
| FABP4 | fatty acid binding protein 4 |
| FASN | fatty acid synthase |
| LCAT | lecithin-cholesterol acyltransferase |
| LDLR | low density lipoprotein receptor |
| LIPC | lipase C, hepatic type |
| LIPG | lipase G, endothelial type |
| LPL | lipoprotein lipase |
| NR1H3 | nuclear receptor subfamily 1 group H |
| | member 3 (liver X receptor alpha) |
| PPARA | peroxisome proliferator activated receptor |
| | alpha |
| PPARG | peroxisome proliferator activated receptor |
| | gamma |
| SCD | stearoyl-CoA desaturase |
| SREBF1 | sterol regulatory element binding |
| | transcription factor 1 |
| VLDLR | very low density lipoprotein receptor |

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “about” means, in general, within a standard deviation of the stated value as determined using a standard analytical technique for measuring the stated value. The terms can also be used by referring to plus or minus 5% of the stated value.

The phrase “effective amount” or “therapeutically effective” means a dosage of a drug or agent sufficient to produce a desired result. The desired result can be subjective or objective improvement in the recipient of the dosage, increased lung growth, increased lung repair, reduced tissue edema, increased DNA repair, decreased apoptosis, a decrease in tumor size, a decrease in the rate of growth of cancer cells, a decrease in metastasis, or any combination of the above.

“Subject” and “patient” refer to either a human or non-human, such as primates, mammals, and vertebrates. In particular embodiments, the subject is a human.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of a tumor or malignancy, delay or slowing of tumor growth and/or metastasis, and an increased lifespan as compared to that expected in the absence of treatment.

“Chromatin” as used herein refers to an organized complex of chromosomal DNA associated with histones.

“Cis-regulatory elements” or “CREs” as used interchangeably herein refers to regions of non-coding DNA which regulate the transcription of nearby genes. CREs are found in the vicinity of the gene, or genes, they regulate. CREs typically regulate gene transcription by functioning as binding sites for transcription factors. Examples of CREs include promoters and enhancers.

“Clustered Regularly Interspaced Short Palindromic Repeats” and “CRISPRs”, as used interchangeably herein refers to loci containing multiple short direct repeats that are found in the genomes of approximately 40% of sequenced bacteria and 90% of sequenced archaea.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimize.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

“Endogenous gene” as used herein refers to a gene that originates from within an organism, tissue, or cell. An endogenous gene is native to a cell, which is in its normal genomic and chromatin context, and which is not heterologous to the cell. Such cellular genes include, e.g., animal genes, plant genes, bacterial genes, protozoal genes, fungal genes, mitochondrial genes, and chloroplastic genes.

“Enhancer” as used herein refers to non-coding DNA sequences containing multiple activator and repressor binding sites. Enhancers range from 200 bp to 1 kb in length and may be either proximal, 5′ upstream to the promoter or within the first intron of the regulated gene, or distal, in introns of neighboring genes or intergenic regions faraway from the locus. Through DNA looping, active enhancers contact the promoter dependently of the core DNA binding motif promoter specificity, 4 to 5 enhancers may interact with a promoter. Similarly, enhancers may regulate more than one gene without linkage restriction and may “skip” neighboring genes to regulate more distant ones. Transcriptional regulation may involve elements located in a chromosome different to one where the promoter resides. Proximal enhancers or promoters of neighboring genes may serve as platforms to recruit more distal elements.

“Fusion protein” as used herein refers to a chimeric protein created through the joining of two or more genes that originally coded for separate proteins. The translation of the fusion gene results in a single polypeptide with functional properties derived from each of the original proteins.

“Genetic construct” as used herein refers to the DNA or RNA molecules that comprise a nucleotide sequence that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Histone acetyltransferases” or “HATs” are used interchangeably herein refers to enzymes that acetylate conserved lysine amino acids on histone proteins by transferring an acetyl group from acetyl CoA to form ε-N-acetyllysine. DNA is wrapped around histones, and, by transferring an acetyl group to the histones, genes can be turned on and off. In general, histone acetylation increases gene expression as it is linked to transcriptional activation and associated with euchromatin Histone acetyltransferases can also acetylate non-histone proteins, such as nuclear receptors and other transcription factors to facilitate gene expression.

“Identical” or “identity” as used herein in the context of two or more nucleic acids or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA. RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

“p300 protein” as used herein refers to the adenovirus E1A-associated cellular p300 transcriptional co-activator protein encoded by the EP300 gene, p300 is a highly conserved acetyltransferase involved in a wide range of cellular processes, p300 functions as a histone acetyltransferase that regulates transcription via chromatin remodeling and is involved with the processes of cell proliferation and differentiation.

“Promoter” as used herein means a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

“Target enhancer” as used herein refers to enhancer that is targeted by a gRNA and CRISPR/Cas9-based gene activation system. The target enhancer may be within the target region.

“Target gene” as used herein refers to any nucleotide sequence encoding a known or putative gene product. The target gene includes the regulatory regions, such as the promoter and enhancer regions, the transcribed regions, which include the coding regions, and other function sequence regions.

“Target region” as used herein refers to a cis-regulatory region or a trans-regulatory region of a target gene to which the guide RNA is designed to recruit the CRISPR/Cas9-based gene activation system to modulate the epigenetic structure and allow the activation of gene expression of the target gene.

“Target regulatory element” as used herein refers to a regulatory element that is targeted by a gRNA and CRISPR/Cas9-based gene activation system. The target regulatory element may be within the target region.

“Transcribed region” as used herein refers to the region of DNA that is transcribed into single-stranded RNA molecule, known as messenger RNA, resulting in the transfer of genetic information from the DNA molecule to the messenger RNA. During transcription, RNA polymerase reads the template strand in the 3′ to 5′ direction and synthesizes the RNA from 5′ to 3′. The mRNA sequence is complementary to the DNA strand.

“Transcriptional Start Site” or “TSS” as used interchangeably herein refers to the first nucleotide of a transcribed DNA sequence where RNA polymerase begins synthesizing the RNA transcript.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism's genetic code. The introduction of a transgene has the potential to change the phenotype of an organism.

“Trans-regulatory elements” as used herein refers to regions of non-coding DNA which regulate the transcription of genes distant from the gene from which they were transcribed. Trans-regulatory elements may be on the same or different chromosome from the target gene.

“Variant” used herein with respect to a nucleic acid means (i) a portion or fragment of a referenced nucleotide sequence, (ii) the complement of a referenced nucleotide sequence or portion thereof; (iii) a nucleic acid that is substantially identical to a referenced nucleic acid or the complement thereof, or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes may be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes may be substituted and still retain protein function. In one aspect, amino acids having hydropathic indexes of +2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within +2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector may be a viral vector, bacteriophage, bacterial artificial chromosome or yeast artificial chromosome. A vector may be a DNA or RNA vector. A vector may be a self-replicating extrachromosomal vector, and preferably, is a DNA plasmid.

### II. ENGINEERED LYSINE ACYLTRANSFERASE

Provided herein is an engineered lysine acyltransferase, such as lysine acetyltransferase (e.g., p300 or CBP), which has at least one (e.g., 2, 3, 4, 5 or more) amino acid mutation with respect to endogenous lysine acyltransferase, such as acetyltransferase. The one or more mutations may flank the ac) 1-CoA binding site (across the entirety of the scanned region 1381-1420) but outside of the 1398-1399 residues that are known catalytic mutants can be engineered to modulate expression and activity of the enzyme. In some aspects, the one or more mutations are in the beta strands composing residues 1383-1385 and 1392-1400 along with the linker region between these. In certain aspects, the one or more mutations are in the helices formed by residues 1407-1409 and 1410-1428. These regions are purported to govern the conformation of the acetyl-CoA binding pocket and may govern the recognition of protein substrates. By changing the interactions with either the acyl-CoA molecule in the acyl portion or the CoA portion, proteins were selectively enriched for certain acyl substrates or globally effecting recognition by altering CoA binding, respectively. This aspect is what drives the expression and activity of the enzyme. In particular aspects, the one or more mutations may be at hydrophobic (L, Y, I) or structurally distinct residues (P. C). For example, the mutations may comprise I1417N, I1395G, P1388C, Y1397Q, Y1397A, and/or C1385Q. Exemplary engineered acyltransferases are provided below. The present engineered acyltransferases may comprise a sequence having at least 80%, 85%, 90%, 91%, 92%, 93%, 94%, 95%, 96%, 97%, 98%, 99%, or 100% sequence similarity to SEQ ID Nos: 1-8. The acyltransferase may comprise p300 having at least 80%, 85%, 90%, 91%, 92%, 93%, 94%, 95%, 96%, 97%, 98%, 99%, or 100% sequence similarity to SEQ ID NO: 9 and/or 1, 2, 3, 4, or 5 point mutations to SEQ ID NO: 9. The acyltransferase may comprise a nuclear localization sequence having at least 80%, 85%, 90%, 91%, 92%, 93%, 94%, 95%, 96%, 97%, 98%, 99%, or 100% sequence similarity to SEQ ID NO: 10 (PKKKRKV) or SEQ ID NO: 11 (KRPAATKKAGQAKKKK). The acyltransferase may comprise a glycine-serine linker having at least 80%, 85%, 90%, 91%, 92%, 93%, 94%, 95%, 96%, 97%, 98%, 99%, or 100% sequence similarity to SEQ ID NO: 12 (GGSGGSGGSGGSGGS) and/or an entry site having at least 80%, 85%, 90%, 91%, 92%, 93%, 94%, 95%, 96%, 97%, 98%, 99%, or 100% sequence similarity to SEQ ID NO: 13 (ASALSPSLIN).

SpdCas9-entry: amino acid sequence;
Streptococcus pyogenes Cas9
(D10A, H840A), Nuclear Localization Sequence,
Glycine-Serine Linker, Entry Site
SEQ ID NO: 1
MDYKDDDDKPKKKRKVTGSRALSGGGSGGSGSDKKYSIGLAIGTNSVGWA
VITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFL
IEGDLNPDNSDVDKLFIQLVQTYNQLFEENPINASGVDAKAILSARLSKS
RRLENLIAQLPGEKKNGLFGNLIALSLGLTPNFKSNEDLABDAKLQLSKD
TYDDDLDNLLAQIGDQYADLFLAAKNLSDAILLSDILRVNTEITKAPLSA
SMIKRYDEHHQDLTLLKALVRQQLPEKYKEIFFDQSKNGYAGYIDGGASQ
EEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKS
EETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDI
VLTLTLFEDREMIEERLKTYAHLFDDKVMKQLKRRRYTGWGRLSRKLING
IRDKQSGKTILDFLKSDGFANRNFMQLIHDDSLTFKEDIQKAQVSGQGDS
LHEHIANLAGSPAIKKGILQTVKVVDELVKVMGRHKPENIVIEMARENQT
TQKGQKNSRERMKRIEEGIKELGSQILKEHPVENTQLQNEKLYLYYLQNG
RDMYVDQELDINRLSDYDVDAIVPQSFLKDDSIDNKVLTRSDKNRGKSDN
VPSEEVVKKMKNYWRQLLNAKLITQRKFDNLTKAERGGLSELDKAGFIKR
QLVETRQITKHVAQILDSRMNTKYDENDKLIREVKVITLKSKLVSDFRKD
FQFYKVREINNYHHAHDAYLNAVVGTALIKKYPKLESEFVYGDYKVYDVR
KMIAKSEQEIGKATAKYFFYSNIMNFFKTEITLANGEIRKRPLIETNGET
GEIVWDKGRDFATVRKVLSMPQVNIVKKTEVQTGGFSKESILPKRNSDKL
IARKKDWDPKKYGGFDSPTVAYSVLVVAKVEKGKSKKLKSVKELLGITIM
ERSSFEKNPIDFLEAKGYKEVKKDLIIKLPKYSLFELENGRKRMLASAGE
LQKGNELALPSKYVNFLYLASHYEKLKGSPEDNEQKQLFVEQHKHYLDEI
IEQISEFSKRVILADANLDKVLSAYNKHRDKPIREQAENIIHLFTLTNLG
APAAFKYFDTTIDRKRYTSTKEVLDATLIHQSITGLYETRIDLSQLGGDK
RPAATKKAGQAKKKKGGSGGSGGSGGSGGSVVSASALSPSLIN
SpdCas9-p300 WT: Streptococcus pyogenes
Cas9 (D10A, H840A),
Nuclear Localization Sequence,
Glycine-Serine Linker, p300
SEQ ID NO: 2
MDYKDDDDKPKKKRKVTGSRALSGGGSGGSGSDKKYSIGLAIGTNSVGWA
VITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFL
IEGDLNPDNSDVDKLFIQLVQTYNQLFEENPINASGVDAKAILSARLSKS
RRLENLIAQLPGEKKNGLFGNLIALSLGLTPNFKSNFDLAEDAKLQLSKD
TYDDDLDNLLAQIGDQYADLFLAAKNLSDAILLSDILRVNTEITKAPLSA
SMIKRYDEHHQDLTLLKALVRQQLPEKYKEIFFDQSKNGYAGYIDGGASQ
EEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKS
EETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDI
VLTLTLFEDREMIEERLKTYAHLFDDKVMKQLKRRRYTGWGRLSRKLING
IRDKQSGKTILDFLKSDGFANRNFMQLIHDDSLTFKEDIQKAQVSGQGDS
LHEHIANLAGSPAIKKGILQTVKVVDELVKVMGRHKPENIVIEMARENQT
TQKGQKNSRERMKRIEEGIKELGSQILKEHPVENTQLQNEKLYLYYLQNG
RDMYVDQELDINRLSDYDVDAIVPQSFLKDDSIDNKVLTRSDKNRGKSDN
VPSEEVVKKMKNYWRQLLNAKLITQRKFDNLTKAERGGLSELDKAGFIKR
QLVETRQITKHVAQILDSRMNTKYDENDKLIREVKVITLKSKLVSDFRKD
FQFYKVREINNYHHAHDAYLNAVVGTALIKKYPKLESEFVYGDYKVYDVR
KMIAKSEQEIGKATAKYFFYSNIMNFFKTEITLANGEIRKRPLIETNGET
GEIVWDKGRDFATVRKVLSMPQVNIVKKTEVQTGGFSKESILPKRNSDKL
IARKKDWDPKKYGGFDSPTVAYSVLVVAKVEKGKSKKLKSVKELLGITIM
ERSSFEKNPIDFLEAKGYKEVKKDLIIKLPKYSLFELENGRKRMLASAGE
LQKGNELALPSKYVNFLYLASHYEKLKGSPEDNEQKQLFVEQHKHYLDEI
IEQISEFSKRVILADANLDKVLSAYNKHRDKPIREQAENIIHLFTLINLG
APAAFKYFDTTIDRKRYTSTKEVLDATLIHQSITGLYETRIDLSQLGGDK
RPAATKKAGQAKKKKGGSGGSGGSGGSGGSVVSIFKPEELRQALMPTLEA
LYRQDPESLPFRQPVDPQLLGIPDYFDIVKSPMDLSTIKRKLDTGQYQEP
WQYVDDIWLMFNNAWLYNRKTSRVYKYCSKLSEVFEQEIDPVMQSLGYCC
GRKLEFSPQTLCCYGKQLCTIPRDATYYSYQNRYHFCEKCFNEIQGESVS
LGDDPSQPQTTINKEQFSKRKNDTLDPELFVECTECGRKMHQICVLHHEI
IWPAGFVCDGCLKKSARTRKENKFSAKRLPSTRLGTFLENRVNDFLRRQN
HPESGEVTVRVVHASDKTVEVKPGMKARFVDSGEMAESFPYRTKALFAFE
EIDGVDLCFFGMHVQEYGSDCPPPNQRRVYISYLDSVHFFRPKCLRTAVY
HEILIGYLEYVKKLGYTTGHIWACPPSEGDDYIFHCHPPDQKIPKPKRLQ
EWYKKMLDKAVSERIVHDYKDIFKQATEDRLTSAKELPYFEGDFWPNVLE
ESIKELEQEEEERKREENTSNESTDVTKGDSKNAKKKNNKKTSKNKSSLS
RGNKKKPGMPNVSNDLSQKLYATMEKHKEVFFVIRLIAGPAANSLPPIVD
PDPLIPCDLMDGRDAFLTLARDKHLEFSSLRRAQWSTMCMLVELHTQSQD
SpdCas9-p300 11395G: amino acid sequence;
Streptococcus pyogenes
Cas9 (D10A, H840A), Nuclear Localization Sequence,
Glycine-Serine Linker, p300
(I1395G)
SEQ ID NO: 3
MDYKDDDDKPKKKRKVTGSRALSGGGSGGSGSDKKYSIGLAIGTNSVGWA
VITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFL
IEGDLNPDNSDVDKLFIQLVQTYNQLFEENPINASGVDAKAILSARLSKS
RRLENLIAQLPGEKKNGLFGNLIALSLGLTPNFKSNEDLAEDAKLQLSKD
TYDDDLDNLLAQIGDQYADLFLAAKNLSDAILLSDILRVNTEITKAPLSA
SMIKRYDEHHQDLTLLKALVRQQLPEKYKEIFFDQSKNGYAGYIDGGASQ
EEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKS
EETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDI
VLTLTLFEDREMIEERLKTYAHLEDDKVMKQLKRRRYTGWGRLSRKLING
IRDKQSGKTILDFLKSDGFANRNFMQLIHDDSLTFKEDIQKAQVSGQGDS
LHEHIANLAGSPAIKKGILQTVKVVDELVKVMGRHKPENIVIEMARENQT
TQKGQKNSRERMKRIEEGIKELGSQILKEHPVENTQLQNEKLYLYYLQNG
RDMYVDQELDINRLSDYDVDAIVPQSFLKDDSIDNKVLTRSDKNRGKSDN
VPSEEVVKKMKNYWRQLLNAKLITQRKFDNLTKAERGGLSELDKAGFIKR
QLVETRQITKHVAQILDSRMNTKYDENDKLIREVKVITLKSKLVSDFRKD
FQFYKVREINNYHHAHDAYLNAVVGTALIKKYPKLESEFVYGDYKVYDVR
KMIAKSEQEIGKATAKYFFYSNIMNFFKTEITLANGEIRKRPLIETNGET
GEIVWDKGRDFATVRKVLSMPQVNIVKKTEVQTGGFSKESILPKRNSDKL
IARKKDWDPKKYGGFDSPTVAYSVLVVAKVEKGKSKKLKSVKELLGITIM
ERSSFEKNPIDFLEAKGYKEVKKDLIIKLPKYSLFELENGRKRMLASAGE
LQKGNELALPSKYVNFLYLASHYEKLKGSPEDNEQKQLFVEQHKHYLDEI
IEQISEFSKRVILADANLDKVLSAYNKHRDKPIREQAENIIHLFTLTNLG
APAAFKYFDTTIDRKRYTSTKEVLDATLIHQSITGLYETRIDLSQLGGDK
RPAATKKAGQAKKKKGGSGGSGGSGGSGGSVVSIFKPEELRQALMPTLEA
LYRQDPESLPFRQPVDPQLLGIPDYFDIVKSPMDLSTIKRKLDTGQYQEP
WQYVDDIWLMFNNAWLYNRKTSRVYKYCSKLSEVFEQEIDPVMQSLGYCC
GRKLEFSPQTLCCYGKQLCTIPRDATYYSYQNRYHFCEKCFNEIQGESVS
LGDDPSQPQTTINKEQFSKRKNDTLDPELFVECTECGRKMHQICVLHHEI
IWPAGFVCDGCLKKSARTRKENKFSAKRLPSTRLGTFLENRVNDFLRRQN
HPESGEVTVRVVHASDKTVEVKPGMKARFVDSGEMAESFPYRTKALFAFE
EIDGVDLCFFGMHVQEYGSDCPPPNQRRVYGSYLDSVHFFRPKCLRTAVY
HEILIGYLEYVKKLGYTTGHIWACPPSEGDDYIFHCHPPDQKIPKPKRLQ
EWYKKMLDKAVSERIVHDYKDIFKQATEDRLTSAKELPYFEGDFWPNVLE
ESIKELEQEEEERKREENTSNESTDVTKGDSKNAKKKNNKKTSKNKSSLS
RGNKKKPGMPNVSNDLSQKLYATMEKHKEVFFVIRLIAGPAANSLPPIVD
PDPLIPCDLMDGRDAFLTLARDKHLEFSSLRRAQWSTMCMLVELHTQSQD
SpdCas9-p300 11417N: amino acid sequence;
Streptococcus pyogenes
Cas9 (D10A, H840A), Nuclear Localization
Sequence, Glycine-Serine Linker, p300
(11417N)
SEQ ID NO: 4
MDYKDDDDKPKKKRKVTGSRALSGGGSGGSGSDKKYSIGLAIGTNSVGWA
VITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFL
IEGDLNPDNSDVDKLFIQLVQTYNQLFEENPINASGVDAKAILSARLSKS
RRLENLIAQLPGEKKNGLFGNLIALSLGLTPNFKSNFDLAEDAKLQLSKD
TYDDDLDNLLAQIGDQYADLFLAAKNLSDAILLSDILRVNTEITKAPLSA
SMIKRYDEHHQDLTLLKALVRQQLPEKYKEIFFDQSKNGYAGYIDGGASQ
EEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKS
EETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDI
VLTLTLFEDREMIEERLKTYAHLFDDKVMKQLKRRRYTGWGRLSRKLING
IRDKQSGKTILDFLKSDGFANRNFMQLIHDDSLTFKEDIQKAQVSGQGDS
LHEHIANLAGSPAIKKGILQTVKVVDELVKVMGRHKPENIVIEMARENQT
TQKGQKNSRERMKRIEEGIKELGSQILKEHPVENTQLQNEKLYLYYLQNG
RDMYVDQELDINRLSDYDVDAIVPQSFLKDDSIDNKVLTRSDKNRGKSDN
VPSEEVVKKMKNYWRQLLNAKLITQRKFDNLTKAERGGLSELDKAGFIKR
QLVETRQITKHVAQILDSRMNTKYDENDKLIREVKVITLKSKLVSDFRKD
FQFYKVREINNYHHAHDAYLNAVVGTALIKKYPKLESEFVYGDYKVYDVR
KMIAKSEQEIGKATAKYFFYSNIMNFFKTEITLANGEIRKRPLIETNGET
GEIVWDKGRDFATVRKVLSMPQVNIVKKTEVQTGGFSKESILPKRNSDKL
IARKKDWDPKKYGGFDSPTVAYSVLVVAKVEKGKSKKLKSVKELLGITIM
ERSSFEKNPIDFLEAKGYKEVKKDLIIKLPKYSLFELENGRKRMLASAGE
LQKGNELALPSKYVNFLYLASHYEKLKGSPEDNEQKQLFVEQHKHYLDEI
IEQISEFSKRVILADANLDKVLSAYNKHRDKPIREQAENIIHLFTLTNLG
APAAFKYFDTTIDRKRYTSTKEVLDATLIHQSITGLYETRIDLSQLGGDK
RPAATKKAGQAKKKKGGSGGSGGSGGSGGSVVSIFKPEELRQALMPTLEA
LYRQDPESLPFRQPVDPQLLGIPDYFDIVKSPMDLSTIKRKLDTGQYQEP
WQYVDDIWLMFNNAWLYNRKTSRVYKYCSKLSEVFEQEIDPVMQSLGYCC
GRKLEFSPQTLCCYGKQLCTIPRDATYYSYQNRYHFCEKCFNEIQGESVS
LGDDPSQPQTTINKEQFSKRKNDTLDPELFVECTECGRKMHQICVLHHEI
IWPAGFVCDGCLKKSARTRKENKFSAKRLPSTRLGTFLENRVNDFLRRQN
HPESGEVTVRVVHASDKTVEVKPGMKARFVDSGEMAESFPYRTKALFAFE
EIDGVDLCFFGMHVQEYGSDCPPPNQRRVYISYLDSVHFFRPKCLRTAVY
HEILIGYLEYVKKLGYTTGHIWACPPSEGDDYIFHCHPPDQKIPKPKRLQ
EWYKKMLDKAVSERIVHDYKDIFKQATEDRLTSAKELPYFEGDFWPNVLE
ESIKELEQEEEERKREENTSNESTDVTKGDSKNAKKKNNKKTSKNKSSLS
RGNKKKPGMPNVSNDLSQKLYATMEKHKEVFFVIRLIAGPAANSLPPIVD
PDPLIPCDLMDGRDAFLTLARDKHLEFSSLRRAQWSTMCMLVELHTQSQD
SpdCas9-p300 D1399Y: amino acid sequence;
Streptococcus pyogenes
Cas9 (D10A, H840A), Nuclear Localization
Sequence, Glycine-Serine Linker, p300
(D1399Y)
SEQ ID NO: 5
MDYKDDDDKPKKKRKVTGSRALSGGGSGGSGSDKKYSIGLAIGTNSVGWA
VITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFL
IEGDLNPDNSDVDKLFIQLVQTYNQLFEENPINASGVDAKAILSARLSKS
RRLENLIAQLPGEKKNGLFGNLIALSLGLTPNFKSNEDLAEDAKLQLSKD
TYDDDLDNLLAQIGDQYADLFLAAKNLSDAILLSDILRVNTEITKAPLSA
SMIKRYDEHHQDLTLLKALVRQQLPEKYKEIFFDQSKNGYAGYIDGGASQ
EEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKS
EETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDI
VLTLTLFEDREMIEERLKTYAHLFDDKVMKQLKRRRYTGWGRLSRKLING
IRDKQSGKTILDFLKSDGFANRNFMQLIHDDSLTFKEDIQKAQVSGQGDS
LHEHIANLAGSPAIKKGILQTVKVVDELVKVMGRHKPENIVIEMARENQT
TQKGQKNSRERMKRIEEGIKELGSQILKEHPVENTQLQNEKLYLYYLQNG
RDMYVDQELDINRLSDYDVDAIVPQSFLKDDSIDNKVLTRSDKNRGKSDN
VPSEEVVKKMKNYWRQLLNAKLITQRKFDNLTKAERGGLSELDKAGFIKR
QLVETRQITKHVAQILDSRMNTKYDENDKLIREVKVITLKSKLVSDFRKD
FQFYKVREINNYHHAHDAYLNAVVGTALIKKYPKLESEFVYGDYKVYDVR
KMIAKSEQEIGKATAKYFFYSNIMNFFKTEITLANGEIRKRPLIETNGET
GEIVWDKGRDFATVRKVLSMPQVNIVKKTEVQTGGFSKESILPKRNSDKL
IARKKDWDPKKYGGFDSPTVAYSVLVVAKVEKGKSKKLKSVKELLGITIM
ERSSFEKNPIDFLEAKGYKEVKKDLIIKLPKYSLFELENGRKRMLASAGE
LQKGNELALPSKYVNFLYLASHYEKLKGSPEDNEQKQLFVEQHKHYLDEI
IEQISEFSKRVILADANLDKVLSAYNKHRDKPIREQAENIIHLFTLTNLG
APAAFKYFDTTIDRKRYTSTKEVLDATLIHQSITGLYETRIDLSQLGGDK
RPAATKKAGQAKKKKGGSGGSGGSGGSGGSVVSIFKPEELRQALMPTLEA
LYRQDPESLPFRQPVDPQLLGIPDYFDIVKSPMDLSTIKRKLDTGQYQEP
WQYVDDIWLMFNNAWLYNRKTSRVYKYCSKLSEVFEQEIDPVMQSLGYCC
GRKLEFSPQTLCCYGKQLCTIPRDATYYSYQNRYHFCEKCFNEIQGESVS
LGDDPSQPQTTINKEQFSKRKNDTLDPELFVECTECGRKMHQICVLHHEI
IWPAGFVCDGCLKKSARTRKENKFSAKRLPSTRLGTFLENRVNDFLRRQN
HPESGEVTVRVVHASDKTVEVKPGMKARFVDSGEMAESFPYRTKALFAFE
EIDGVDLCFFGMHVQEYGSDCPPPNQRRVYISYLYSVHFFRPKCLRTAVY
HEILIGYLEYVKKLGYTTGHIWACPPSEGDDYIFHCHPPDQKIPKPKRLQ
EWYKKMLDKAVSERIVHDYKDIFKQATEDRLTSAKELPYFEGDFWPNVLE
ESIKELEQEEEERKREENTSNESTDVTKGDSKNAKKKNNKKTSKNKSSLS
RGNKKKPGMPNVSNDLSQKLYATMEKHKEVFFVIRLIAGPAANSLPPIVD
PDPLIPCDLMDGRDAFLTLARDKHLEFSSLRRAQWSTMCMLVELHTQSQD
rTetR-p300 entry: amino acid sequence; rTelR
(G72P), 3X FLAG epitope,
Glycine-Serine Linker, p300 (Entry Site)
SEQ ID NO: 6
MSRLDKSKIINGALELLNGVGIEGLTTRKLAQKLGVEQPTLYWHVKNKRA
LLDALPIEMLDRHHTHSCPLEPESWQDFLRNNAKSYRCALLSHRDGAKVH
LGTRPTEKQYETLENQLAFLCQQGFSLENALYALSAVGHFTLGCVLEEQE
HQVAKEERETPTTDSMPPLLKQAIELFDRQGAEPAFLFGLELICGLEKQL
KCESGGDYKDHDGDYKDHDIDYKDDDDKGGSGSGSIFKPEELRQALMPTL
EALYRQDPESLPFRQPVDPQLLGIPDYFDIVKSPMDLSTIKRKLDTGQYQ
EPWQYVDDIWLMFNNAWLYNRKTSRVYKYCSKLSEVFEQEIDPVMQSLGY
CCGRKLEFSPQTLCCYGKQLCTIPRDATYYSYQNRYHFCEKCFNEIQGES
VSLGDDPSQPQTTINKEQFSKRKNDTLDPELFVECTECGRKMHQICVLHH
EIIWPAGFVCDGCLKKSARTRKENKFSAKRLPSTRLGTFLENRVNDFLRR
QNHPESGEVTVRVVHASDKTVEVKPGMKARFVDSGEMAESFPYRTKALFA
FEEIDGVDLCFFGMHVQEGDGSSVSYLEYVKKLGYTTGHIWACPPSEGDD
YIFHCHPPDQKIPKPKRLQEWYKKMLDKAVSERIVHDYKDIFKQATEDRL
TSAKELPYFEGDFWPNVLEESIKELEQEEEERKREENTSNESTDVTKGDS
KNAKKKNNKKTSKNKSSLSRGNKKKPGMPNVSNDLSQKLYATMEKHKEVF
FVIRLIAGPAANSLPPIVDPDPLIPCDLMDGRDAFLTLARDKHLEFSSLR
RAQWSTMCMLVELHTQSQDGSGEGRGSLL
dCasMINI-p300 WT: amino acid sequence;
dCasMINI (D10A, H840A),
Nuclear Localization Sequence, Glycine-Serine
Linker, 1X HA Epitope,p300
SEQ ID NO: 7
MGPKKKRKVGSGSAKNTITKTLKLRIVRPYNSAEVEKIVADEKNNREKIA
LEKNKDKVKEACSKHLKVAAYCTTQVERNACLFCKARKLDDKFYQKLRGQ
FPDAVFWQEISEIFRQLQKQAAEIYNQSLIELYYEIFIKGKGIANASSVE
HYLSRVCYRRAAELFKNAAIASGLRSKIKSNFRLKELKNMKSGLPTTKSD
NFPIPLVKQKGGQYTGFEISNHNSDFIIKIPFGRWQVKKEIDKYRPWEKF
DFEQVQKSPKPISLLLSTQRRKRNKGWSKDEGTEAEIKKVMNGDYQTSYI
EVKRGSKICEKSAWMLNLSIDVPKIDKGVDPSIIGGIAVGVRSPLVCAIN
NAFSRYSISDNDLFHFNKKMFARRRILLKKNRHKRAGHGAKNKLKPITIL
TEKSERFRKKLIERWACEIADFFIKNKVGTVQMENLESMKRKEDSYFNIR
LRGFWPYAEMQNKIEFKLKQYGIEIRKVAPNNTSKTCSKCGHLNNYFNFE
YRKKNKFPHFKCEKCNFKENAAYNAALNISNPKLKSTKERPAYPYDVPDY
AGGSGGSGGSVVSIFKPEELRQALMPTLEALYRQDPESLPFRQPVDPQLL
GIPDYFDIVKSPMDLSTIKRKLDTGQYQEPWQYVDDIWLMENNAWLYNRK
TSRVYKYCSKLSEVFEQEIDPVMQSLGYCCGRKLEFSPQTLCCYGKQLCT
IPRDATYYSYQNRYHFCEKCFNEIQGESVSLGDDPSQPQTTINKEQFSKR
KNDTLDPELFVECTECGRKMHQICVLHHEIIWPAGFVCDGCLKKSARTRK
ENKFSAKRLPSTRLGTFLENRVNDFLRRQNHPESGEVTVRVVHASDKTVE
VKPGMKARFVDSGEMAESFPYRTKALFAFEEIDGVDLCFFGMHVQEYGSD
CPPPNQRRVYISYLDSVHFFRPKCLRTAVYHEILIGYLEYVKKLGYTTGH
IWACPPSEGDDYIFHCHPPDQKIPKPKRLQEWYKKMLDKAVSERIVHDYK
DIFKQATEDRLTSAKELPYFEGDFWPNVLEESIKELEQEEEERKREENTS
NESTDVTKGDSKNAKKKNNKKTSKNKSSLSRGNKKKPGMPNVSNDLSQKL
YATMEKHKEVFFVIRLIAGPAANSLPPIVDPDPLIPCDLMDGRDAFLTLA
RDKHLEFSSLRRAQWSTMCMLVELHTQSQD
AsdCas12a-p300 WT: amino acid sequence,
AsCas12a (H800A, E993A),
Nuclear Localization Sequence,
Glycine-Serine Linker, p300, 3X HA Epitope,
SEQ ID NO: 8
MTQFEGFTNLYQVSKTLRFELIPQGKTLKHIQEQGFIEEDKARNDHYKEL
KPIIDRIYKTYADQCLQLVQLDWENLSAAIDSYRKEKTEETRNALIEEQA
TYRNAIHDYFIGRTDNLTDAINKRHAEIYKGLFKAELFNGKVLKQLGTVT
TTEHENALLRSFDKFTTYFSGFYENRKNVFSAEDISTAIPHRIVQDNFPK
FKENCHIFTRLITAVPSLREHFENVKKAIGIFVSTSIEEVFSFPFYNQLL
TQTQIDLYNQLLGGISREAGTEKIKGLNEVLNLAIQKNDETAHIIASLPH
RFIPLFKQILSDRNTLSFILEEFKSDEEVIQSFCKYKTLLRNENVLETAE
ALFNELNSIDLTHIFISHKKLETISSALCDHWDTLRNALYERRISELTGK
ITKSAKEKVQRSLKHEDINLQEIISAAGKELSEAFKQKTSEILSHAHAAL
DQPLPTTLKKQEEKEILKSQLDSLLGLYHLLDWFAVDESNEVDPEFSARL
TGIKLEMEPSLSFYNKARNYATKKPYSVEKFKLNFQMPTLASGWDVNKEK
NNGAILFVKNGLYYLGIMPKQKGRYKALSFEPTEKTSEGFDKMYYDYFPD
AAKMIPKCSTQLKAVTAHFQTHTTPILLSNNFIEPLEITKEIYDLNNPEK
EPKKFQTAYAKKTGDQKGYREALCKWIDFTRDFLSKYTKTTSIDLSSLRP
SSQYKDLGEYYAELNPLLYHISFQRIAEKEIMDAVETGKLYLFQIYNKDF
AKGHHGKPNLHTLYWTGLFSPENLAKTSIKLNGQAELFYRPKSRMKRMAA
RLGEKMLNKKLKDQKTPIPDTLYQELYDYVNHRLSHDLSDEARALLPNVI
TKEVSHEIKDRRFTSDKFFFHVPITLNYQAANSPSKFNQRVNAYLKEHPE
TPIIGIDRGERNLIYITVIDSTGKILEQRSLNTIQQFDYQKKLDNREKER
VAARQAWSVVGTIKDLKQGYLSQVIHEIVDLMIHYQAVVVLANLNFGFKS
KRTGIAEKAVYQQFEKMLIDKLNCLVLKDYPAEKVGGVLNPYQLTDQFTS
FAKMGTQSGFLFYVPAPYTSKIDPLTGFVDPFVWKTIKNHESRKHFLEGF
DFLHYDVKTGDFILHFKMNRNLSFQRGLPGFMPAWDIVFEKNETQFDAKG
TPFIAGKRIVPVIENHRFTGRYRDLYPANELIALLEEKGIVFRDGSNILP
KLLENDDSHAIDTMVALIRSVLQMRNSNAATGEDYINSPVRDLNGVCFDS
RFQNPEWPMDADANGAYHIALKGQLLLNHLKESKDLKLQNGISNQDWLAY
IQELRNKRPAATKKAGQAKKKKGGSGGSGGSGGSGGSVVSIFKPEELRQA
LMPTLEALYRQDPESLPFRQPVDPQLLGIPDYFDIVKSPMDLSTIKRKLD
TGQYQEPWQYVDDIWLMFNNAWLYNRKTSRVYKYCSKLSEVFEQEIDPVM
QSLGYCCGRKLEFSPQTLCCYGKQLCTIPRDATYYSYQNRYHFCEKCFNE
IQGESVSLGDDPSQPQTTINKEQFSKRKNDTLDPELFVECTECGRKMHQI
CVLHHEIIWPAGFVCDGCLKKSARTRKENKFSAKRLPSTRLGTFLENRVN
DFLRRQNHPESGEVTVRVVHASDKTVEVKPGMKARFVDSGEMAESFPYRT
KALFAFEEIDGVDLCFFGMHVQEYGSDCPPPNQRRVYISYLDSVHFFRPK
CLRTAVYHENLIGYLEYVKKLGYTTGHIWACPPSEGDDYIFHCHPPDQKI
PKPKRLQEWYKKMLDKAVSERIVHDYKDIFKQATEDRLTSAKELPYFEGD
FWPNVLEESIKELEQEEEERKREENTSNESTDVTKGDSKNAKKKNNKKTS
KNKSSLSRGNKKKPGMPNVSNDLSQKLYATMEKHKEVFFVIRLIAGPAAN
SLPPIVDPDPLIPCDLMDGRDAFLTLARDKHLEFSSLRRAQWSTMCMLVE
LHTQSQDGSYPYDVPDYAYPYDVPDYAYPYDVPDYA
(p300)
SEQ ID NO: 9
VVSIFKPEELRQALMPTLEALYRQDPESLPFRQPVDPQLLGIPDYFDIVK
SPMDLSTIKRKLDTGQYQEPWQYVDDIWLMFNNAWLYNRKTSRVYKYCSK
LSEVFEQEIDPVMQSLGYCCGRKLEFSPQTLCCYGKQLCTIPRDATYYSY
QNRYHFCEKCFNEIQGESVSLGDDPSQPQTTINKEQFSKRKNDTLDPELF
VECTECGRKMHQICVLHHEIIWPAGFVCDGCLKKSARTRKENKFSAKRLP
STRLGTFLENRVNDFLRRQNHPESGEVTVRVVHASDKTVEVKPGMKARFV
DSGEMAESFPYRTKALFAFEEIDGVDLCFFGMHVQEYGSDCPPPNQRRVY
ISYLDSVHFFRPKCLRTAVYHEILIGYLEYVKKLGYTTGHIWACPPSEGD
DYIFHCHPPDQKIPKPKRLQEWYKKMLDKAVSERIVHDYKDIFKQATEDR
LTSAKELPYFEGDFWPNVLEESIKELEQEEEERKREENTSNESTDVTKGD
SKNAKKKNNKKTSKNKSSLSRGNKKKPGMPNVSNDLSQKLYATMEKHKEV
FFVIRLIAGPAANSLPPIVDPDPLIPCDLMDGRDAFLTLARDKHLEFSSL
RRAQWSTMCMLVELHTQSQD

### A. CRISPR/Cas9-Based Gene Activation System

Certain embodiments of the present disclosure provide a CRISPR/Cas9-based gene activation system for use in activating gene expression of a target gene. The CRISPR/Cas9-based gene activation system includes a fusion protein of a Cas9 protein that does not have nuclease activity, such as dCas9, and an engineered lysine acetyltransferase or lysine acetyltransferase effector domain of the present embodiments. Histone acetylation, carried out by histone acetyltransferases (HATs), plays a fundamental role in regulating chromatin dynamics and transcriptional regulation. The histone acetyltransferase protein releases DNA from its heterochromatin state and allows for continued and robust gene expression by the endogenous cellular machinery. The recruitment of an acetyltransferase by dCas9 to a genomic target site may directly modulate epigenetic structure.

The CRISPR/Cas9-based gene activation system may catalyze acetylation of histone H3 lysine 27 at its target sites, leading to robust transcriptional activation of target genes from promoters and proximal and distal enhancers. The CRISPR/Cas9-based gene activation system is highly specific and may be guided to the target gene using as few as one guide RNA. The CRISPR/Cas9-based gene activation system may activate the expression of one gene or a family of genes by targeting enhancers at distant locations in the genome.

a. CRISPR System

The CRISPR system is a microbial nuclease system involved in defense against invading phages and plasmids that provides a form of acquired immunity. The CRISPR loci in microbial hosts contain a combination of CRISPR-associated (Cas) genes as well as non-coding RNA elements capable of programming the specificity of the CRISPR-mediated nucleic acid cleavage. Short segments of foreign DNA, called spacers, are incorporated into the genome between CRISPR repeats, and serve as a ‘memory’ of past exposures. Cas9 forms a complex with the 3′ end of the single guide RNA (“sgRNA”), and the protein-RNA pair recognizes its genomic target by complementary base pairing between the 5′ end of the sgRNA sequence and a predefined 20 bp DNA sequence, known as the protospacer. This complex is directed to homologous loci of pathogen DNA via regions encoded within the CRISPR RNA (“crRNA”), i.e., the protospacers, and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer). By simply exchanging the 20 bp recognition sequence of the expressed chimeric sgRNA, the Cas9 nuclease can be directed to new genomic targets. CRISPR spacers are used to recognize and silence exogenous genetic elements in a manner analogous to RNAi in eukaryotic organisms.

Three classes of CRISPR systems (Types 1, II and III effector systems) are known. The Type II effector system carries out targeted DNA double-strand break in four sequential steps, using a single effector enzyme, Cas9, to cleave dsDNA. Compared to the Type I and Type III effector systems, which require multiple distinct effectors acting as a complex, the Type II effector system may function in alternative contexts such as eukaryotic cells. The Type II effector system consists of a long pre-crRNA, which is transcribed from the spacer-containing CRISPR locus, the Cas9 protein, and a tracrRNA, which is involved in pre-crRNA processing. The tracrRNAs hybridize to the repeat regions separating the spacers of the pre-crRNA, thus initiating dsRNA cleavage by endogenous RNase III. This cleavage is followed by a second cleavage event within each spacer by Cas9, producing mature crRNAs that remain associated with the tracrRNA and Cas9, forming a Cas9:crRNA-tracrRNA complex.

An engineered form of the Type II effector system of Streptococcus pyogenes was shown to function in human cells for genome engineering. In this system, the Cas9 protein was directed to genomic target sites by a synthetically reconstituted “guide RNA” (“gRNA”, also used interchangeably herein as a chimeric sgRNA, which is a crRNA-tracrRNA fusion that obviates the need for RNase III and crRNA processing in general.

The Cas9:crRNA-tracrRNA complex unwinds the DNA duplex and searches for sequences matching the crRNA to cleave. Target recognition occurs upon detection of complementarity between a “protospacer” sequence in the target DNA and the remaining spacer sequence in the crRNA. Cas9 mediates cleavage of target DNA if a correct protospacer-adjacent motif (PAM) is also present at the 3′ end of the protospacer. For protospacer targeting, the sequence must be immediately followed by the protospacer-adjacent motif (PAM), a short sequence recognized by the Cas9 nuclease that is required for DNA cleavage. Different Type II systems have differing PAM requirements. The S. pyogenes CRISPR system may have the PAM sequence for this Cas9 (SpCas9) as 5′-NRG-3′, where R is either A or G, and characterized the specificity of this system in human cells. A unique capability of the CRISPR/Cas9 system is the straightforward ability to simultaneously target multiple distinct genomic loci by co-expressing a single Cas9 protein with two or more sgRNAs. For example, the Streptococcus pyogenes Type II system naturally prefers to use an “NGG” sequence, where “N” can be any nucleotide, but also accepts other PAM sequences, such as “NAG” in engineered systems (Hsu et al., 2013). Similarly, the Cas9 derived from Neisseria meningitidis (NmCas9) normally has a native PAM of NNNNGATT, but has activity across a variety of PAMs, including a highly degenerate NNNNGNNN PAM (Esvelt et al., 2013).

b. Cas9

The CRISPR/Cas9-based gene activation system may include a Cas9 protein or a Cas9 fusion protein. Cas9 protein is an endonuclease that cleaves nucleic acid and is encoded by the CRISPR loci and is involved in the Type II CRISPR system. The Cas9 protein may be from any bacterial or archaea species, such as Streptococcus pyogenes. Streptococcus thermophiles, or Neisseria meningitides. The Cas9 protein may be mutated so that the nuclease activity is inactivated. In some embodiments, an inactivated Cas9 protein from Streptococcus pyogenes (iCas9, also referred to as “dCas9”) may be used. As used herein, “iCas9” and “dCas9” both refer to a Cas9 protein that has the amino acid substitutions D10A and H840A and has its nuclease activity inactivated. In some embodiments, an inactivated Cas9 protein from Neisseria meningitides, such as NmCas9, may be used.

### c) Histone Acetyltransferase (HAT) Protein

The CRISPR/Cas9-based gene activation system can include the engineered lysine acetyltransferase p300 protein of the present embodiments or fragment thereof. The p300 protein regulates the activity of many genes in tissues throughout the body. The p300 protein plays a role in regulating cell growth and division, prompting cells to mature and assume specialized functions (differentiate) and preventing the growth of cancerous tumors. The p300 protein may activate transcription by connecting transcription factors with a complex of proteins that carry out transcription in the cell's nucleus. The p300 protein also functions as a histone acetyltransferase that regulates transcription via chromatin remodeling.

The engineered p300 may comprise an optimized human p300 protein or a fragment thereof. The lysine acetyltransferase protein may include the core lysine-acetyltransferase domain of the human p300 protein, i.e., the p300 HAT Core (also known as “p300 Core”).

c. gRNA

The CRISPR/Cas9-based gene activation system may include at least one gRNA that targets a nucleic acid sequence. The gRNA provides the targeting of the CRISPR/Cas9-based gene activation system. The gRNA is a fusion of two noncoding RNAs: a crRNA and a tracrRNA. The sgRNA may target any desired DNA sequence by exchanging the sequence encoding a 20 bp protospacer which confers targeting specificity through complementary base pairing with the desired DNA target. gRNA mimics the naturally occurring crRNA tracrRNA duplex involved in the Type 11 Effector system. This duplex, which may include, for example, a 42-nucleotide crRNA and a 75-nucleotide tracrRNA, acts as a guide for the Cas9.

The gRNA may target and bind a target region of a target gene. The target region may be a cis-regulatory region or trans-regulatory region of a target gene. In some embodiments, the target region is a distal or proximal cis-regulatory region of the target gene. The gRNA may target and bind a cis-regulatory region or trans-regulatory region of a target gene. In some embodiments, the gRNA may target and bind an enhancer region, a promoter region, or a transcribed region of a target gene. For example, the gRNA may target and bind the target region is at least one of HS2 enhancer of the human β-globin locus, distal regulatory region (DRR) of the MYOD gene, core enhancer (CE) of the MYOD gene, proximal (PE) enhancer region of the OCT4 gene, or distal (DE) enhancer region of the OCT4 gene. In some embodiments, the target region may be a viral promoter, such as an HIV promoter.

The target region may include a target enhancer or a target regulatory element. In some embodiments, the target enhancer or target regulatory element controls the gene expression of several target genes. In some embodiments, the target enhancer or target regulatory element controls a cell phenotype that involves the gene expression of one or more target genes. In some embodiments, the identity of one or more of the target genes is known. In some embodiments, the identity of one or more of the target genes is unknown. The CRISPR/Cas9-based gene activation system allows the determination of the identity of these unknown genes that are involved in a cell phenotype. Examples of cell phenotypes include, but not limited to, T-cell phenotype, cell differentiation, such as hematopoietic cell differentiation, oncogenesis, immunomodulation, cell response to stimuli, cell death, cell growth, drug resistance, or drug sensitivity.

In some embodiments, at least one gRNA may target and bind a target enhancer or target regulatory element, whereby the expression of one or more genes is activated. For example, between 1 gene and 20 genes, between 1 gene and 15 genes, between 1 gene and 10 genes, between 1 gene and 5 genes, between 2 genes and 20 genes, between 2 genes and 15 genes, between 2 genes and 10 genes, between 2 genes and 5 genes, between 5 genes and 20 genes, between 5 genes and 15 genes, or between 5 genes and 10 genes are activated by at least one gRNA. In some embodiments, at least 1 gene, at least 2 genes, at least 3 genes, at least 4 genes, at least 5 gene, at least 6 genes, at least 7 genes, at least 8 genes, at least 9 gene, at least 10 genes, at least 11 genes, at least 12 genes, at least 13 gene, at least 14 genes, at least 15 genes, or at least 20 genes are activated by at least one gRNA.

The CRISPR/Cas9-based gene activation system may activate genes at both proximal and distal locations relative to the transcriptional start site (TSS). The CRISPR/Cas9-based gene activation system may target a region that is at least about 1 base pair to about 100,000 base pairs, at least about 100 base pairs to about 100,000 base pairs, at least about 250 base pairs to about 100,000 base pairs, at least about 500 base pairs to about 100,000 base pairs, at least about 1,000 base pairs to about 100,000 base pairs, at least about 2,000 base pairs to about 100,000 base pairs, at least about 5,000 base pairs to about 100,000 base pairs, at least about 10,000 base pairs to about 100,000 base pairs, at least about 20,000 base pairs to about 100,000 base pairs, at least about 50,000 base pairs to about 100,000 base pairs, at least about 75,000 base pairs to about 100,000 base pairs, at least about 1 base pair to about 75,000 base pairs, at least about 100 base pairs to about 75,000 base pairs, at least about 250 base pairs to about 75,000 base pairs, at least about 500 base pairs to about 75,000 base pairs, at least about 1,000 base pairs to about 75,000 base pairs, at least about 2,000 base pairs to about 75,000 base pairs, at least about 5,000 base pairs to about 75,000 base pairs, at least about 10,000 base pairs to about 75,000 base pairs, at least about 20,000 base pairs to about 75,000 base pairs, at least about 50,000 base pairs to about 75,000 base pairs, at least about 1 base pair to about 50,000 base pairs, at least about 100 base pairs to about 50,000 base pairs, at least about 250 base pairs to about 50,000 base pairs, at least about 500 base pairs to about 50,000 base pairs, at least about 1,000 base pairs to about 50,000 base pairs, at least about 2,000 base pairs to about 50,000 base pairs, at least about 5,000 base pairs to about 50,000 base pairs, at least about 10,000 base pairs to about 50,000 base pairs, at least about 20,000 base pairs to about 50,000 base pairs, at least about 1 base pair to about 25,000 base pairs, at least about 100 base pairs to about 25,000 base pairs, at least about 250 base pairs to about 25,000 base pairs, at least about 500 base pairs to about 25,000 base pairs, at least about 1,000 base pairs to about 25,000 base pairs, at least about 2,000 base pairs to about 25,000 base pairs, at least about 5,000 base pairs to about 25,000 base pairs, at least about 10,000 base pairs to about 25,000 base pairs, at least about 20,000 base pairs to about 25,000 base pairs, at least about 1 base pair to about 10,000 base pairs, at least about 100 base pairs to about 10,000 base pairs, at least about 250 base pairs to about 10,000 base pairs, at least about 500 base pairs to about 10,000 base pairs, at least about 1,000 base pairs to about 10,000 base pairs, at least about 2,000 base pairs to about 10,000 base pairs, at least about 5,000 base pairs to about 10,000 base pairs, at least about 1 base pair to about 5,000 base pairs, at least about 100 base pairs to about 5,000 base pairs, at least about 250 base pairs to about 5,000 base pairs, at least about 500 base pairs to about 5,000 base pairs, at least about 1,000 base pairs to about 5,000 base pairs, or at least about 2,000 base pairs to about 5,000 base pairs upstream from the TSS. The CRISPR/Cas9-based gene activation system may target a region that is at least about 1 base pair, at least about 100 base pairs, at least about 500 base pairs, at least about 1,000 base pairs, at least about 1.250 base pairs, at least about 2,000 base pairs, at least about 2,250 base pairs, at least about 2,500 base pairs, at least about 5,000 base pairs, at least about 10,000 base pairs, at least about 11,000 base pairs, at least about 20,000 base pairs, at least about 30,000 base pairs, at least about 46,000 base pairs, at least about 50,000 base pairs, at least about 54,000 base pairs, at least about 75,000 base pairs, or at least about 100,000 base pairs upstream from the TSS.

The CRISPR/Cas9-based gene activation system may target a region that is at least about 1 base pair to at least about 500 base pairs, at least about 1 base pair to at least about 250 base pairs, at least about 1 base pair to at least about 200 base pairs, at least about 1 base pair to at least about 100 base pairs, at least about 50 base pairs to at least about 500 base pairs, at least about 50 base pairs to at least about 250 base pairs at least about 50 base pairs to at least about 200 base pairs, at least about 50 base pairs to at least about 100 base pairs, at least about 100 base pairs to at least about 500 base pairs, at least about 100 base pairs to at least about 250 base pairs, or at least about 100 base pairs to at least about 200 base pairs downstream from the TSS. The CRISPR/Cas9-based gene activation system may target a region that is at least about 1 base pair, at least about 2 base pairs, at least about 3 base pairs, at least about 4 base pairs, at least about 5 base pairs, at least about 10 base pairs, at least about 15 base pairs, at least about 20 base pairs, at least about 25 base pairs, at least about 30 base pairs, at least about 40 base pairs, at least about 50 base pairs, at least about 60 base pairs, at least about 70 base pairs, at least about 80 base pairs, at least about 90 base pairs, at least about 100 base pairs, at least about 110 base pairs, at least about 120, at least about 130, at least about 140 base pairs, at least about 150 base pairs, at least about 160 base pairs, at least about 170 base pairs, at least about 180 base pairs, at least about 190 base pairs, at least about 200 base pairs, at least about 210 base pairs, at least about 220, at least about 230, at least about 240 base pairs, or at least about 250 base pairs downstream from the TSS.

In some embodiments, the CRISPR/Cas9-based gene activation system may target and bind a target region that is on the same chromosome as the target gene but more than 100,000 base pairs upstream or more than 250 base pairs downstream from the TSS. In some embodiments, the CRISPR/Cas9-based gene activation system may target and bind a target region that is on a different chromosome from the target gene.

The CRISPR/Cas9-based gene activation system may use gRNA of varying sequences and lengths. The gRNA may comprise a complementary polynucleotide sequence of the target DNA sequence followed by NGG. The gRNA may comprise a “G” at the 5′ end of the complementary polynucleotide sequence. The gRNA may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by NGG. The gRNA may target at least one of the promoter region, the enhancer region or the transcribed region of the target gene.

The CRISPR/Cas9-based gene activation system may include at least 1 gRNA, at least 2 different gRNAs, at least 3 different gRNAs at least 4 different gRNAs, at least 5 different gRNAs, at least 6 different gRNAs, at least 7 different gRNAs, at least 8 different gRNAs, at least 9 different gRNAs, or at least 10 different gRNAs. The CRISPR/Cas9-based gene activation system may include between at least I gRNA to at least 10 different gRNAs, at least 1 gRNA to at least 8 different gRNAs, at least I gRNA to at least 4 different gRNAs, at least 2 gRNA to at least 10 different gRNAs, at least 2 gRNA to at least 8 different gRNAs, at least 2 different gRNAs to at least 4 different gRNAs, at least 4 gRNA to at least 10 different gRNAs, or at least 4 different gRNAs to at least 8 different gRNAs.

d. Target Genes

The CRISPR/Cas9-based gene activation system may be designed to target and activate the expression of any target gene. The target gene may be an endogenous gene, a transgene, or a viral gene in a cell line. In some embodiments, the target region is located on a different chromosome as the target gene. In some embodiments, the CRISPR/Cas9-based gene activation system may include more than 1 gRNA. In some embodiments, the CRISPR/Cas9-based gene activation system may include more than 1 different gRNAs. In some embodiments, the different gRNAs bind to different target regions. For example, the different gRNAs may bind to target regions of different target genes and the expression of two or more target genes are activated.

In some embodiments, the CRISPR/Cas9-based gene activation system may activate between about one target gene to about ten target genes, about one target genes to about five target genes, about one target genes to about four target genes, about one target genes to about three target genes, about one target genes to about two target genes, about two target gene to about ten target genes, about two target genes to about five target genes, about two target genes to about four target genes, about two target genes to about three target genes, about three target genes to about ten target genes, about three target genes to about five target genes, or about three target genes to about four target genes. In some embodiments, the CRISPR/Cas9-based gene activation system may activate at least one target gene, at least two target genes, at least three target genes, at least four target genes, at least five target genes, or at least ten target genes. For example, the may target the hypersensitive site 2 (HS2) enhancer region of the human β-globin locus and activate downstream genes (HBE, HBG, HBD and HBB).

In some embodiments, the CRISPR/Cas9-based gene activation system induces the gene expression of a target gene by at least about 1 fold, at least about 2 fold, at least about 3 fold, at least about 4 fold, at least about 5 fold, at least about 6 fold, at least about 7 fold, at least about 8 fold, at least about 9 fold, at least about 10 fold, at least 15 fold, at least 20 fold, at least 30 fold, at least 40 fold, at least 50 fold, at least 60 fold, at least 70 fold, at least 80 fold, at least 90 fold, at least 100 fold, at least about 110 fold, at least 120 fold, at least 130 fold, at least 140 fold, at least 150 fold, at least 160 fold, at least 170 fold, at least 180 fold, at least 1I90 fold, at least 200 fold, at least about 300 fold, at least 400 fold, at least 500 fold, at least 600 fold, at least 700 fold, at least 800 fold, at least 900 fold, or at least 1000 fold compared to a control level of gene expression. A control level of gene expression of the target gene may be the level of gene expression of the target gene in a cell that is not treated with any CRISPR/Cas9-based gene activation system.

The target gene may be a mammalian gene. For example, the CRISPR/Cas9-based gene activation system may target a mammalian gene, such as IL1RN, MYOD1, OCT4, HBE, HBG, HBD, HBB, MYOCD (Myocardin), PAX7 (Paired box protein Pax-7), FGF1 (fibroblast growth factor-1) genes, such as FGF1A, FGF1B, and FGF1C. Other target genes include, but not limited to, Atf3, Axud1, Btg2, c-Fos, c-Jun, Cxcl1, Cxcl2, Edn1, Ereg, Fos, Gadd45b, Ier2, Ier3, Ifrd1, Il1b, Il6, Irf1, Junb, Lif, Nfkbia, Nfkbiz, Ptgs2, Slc25a25, Sqstm1, Tieg, Tnf, Tnfaip3, Zfp36, Birc2, Ccl2, Ccl20, Ccl7, Cebpd, Ch25h, CSF1, Cx3cl1, Cxcl10, Cxcl5, Gch, Icam1, Ifi47, Ifngr2, Mmp10, Nfkbie, Npal1, p21, Relb, Ripk2, Rnd1, S1pr3, Stx11, Tgtp, Tlr2, Tmem140, Tnfaip2, Tnfrsf6, Vcam1, I110004C05Rik (GenBank accession number BC010291), Abca1, AI561871 (GenBank accession number B1143915), AI882074 (GenBank accession number BB730912), Arts1, AW049765 (GenBank accession number BC026642. 1), C3, Casp4, Ccl5, Ccl9, Cdsn, Enpp2, Gbp2, H2-D1, H2-K, H2-L, Ifit1, Ii, Il13ra1, Il1rl1, Lcn2, Lhfpl2, LOC677168 (GenBank accession number AK019325), Mmp13, Mmp3, Mt2, Naf1, Ppicap, Prnd, Psmb10, Saa3, Serpina3g, Serpinf1, Sod3, Stat1, Tapbp, U90926 (GenBank accession number NM_020562), Ubd, A2AR (Adenosine A2A receptor), B7-H3 (also called CD276), B7-H4 (also called VTCN1), BTLA (B and T Lymphocyte Attenuator; also called CD272), CTLA-4 (Cytotoxic T-Lymphocyte-Associated protein 4; also called CD152). IDO (Indoleamine 2,3-dioxygenase) KIR (Killer-cell Immunoglobulin-like Receptor), LAG3 (Lymphocyte Activation Gene-3), PD-1 (Programmed Death 1 (PD-1) receptor), TIM-3 (T-cell Immunoglobulin domain and Mucin domain 3), and VISTA (V-domain Ig suppressor of T cell activation).

e. Compositions for Gene Activation

Further provided herein is a composition for activating gene expression of a target gene, target enhancer, or target regulatory element in a cell or subject. The composition may include the CRISPR/Cas9-based gene activation system, as disclosed above. The composition may also include a viral delivery system. For example, the viral delivery system may include an adeno-associated virus vector or a modified lentiviral vector.

Methods of introducing a nucleic acid into a host cell are known in the art, and any known method can be used to introduce a nucleic acid (e.g., an expression construct) into a cell. Suitable methods include, include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like. In some embodiments, the composition may be delivered by mRNA delivery and ribonucleoprotein (RNP) complex delivery.

### (i) Constructs and Plasmids

The compositions, as described above, may comprise genetic constructs that encode the CRISPR/Cas9-based gene activation system, as disclosed herein. The genetic construct, such as a plasmid or expression vector, may comprise a nucleic acid that encodes the CRISPR/Cas9-based gene activation system, such as the CRISPR/Cas9-based acetyltransferase and/or at least one of the gRNAs. The compositions, as described above, may comprise genetic constructs that encode the modified AAV vector and a nucleic acid sequence that encodes the CRISPR/Cas9-based gene activation system, as disclosed herein. The genetic construct, such as a plasmid, may comprise a nucleic acid that encodes the CRISPR/Cas9-based gene activation system. The compositions, as described above, may comprise genetic constructs that encode a modified lentiviral vector. The genetic construct, such as a plasmid, may comprise a nucleic acid that encodes the CRISPR/Cas9-based acetyltransferase and at least one sgRNA. The genetic construct may be present in the cell as a functioning extrachromosomal molecule. The genetic construct may be a linear minichromosome including centromere, telomeres or plasmids or cosmids.

The genetic construct may also be part of a genome of a recombinant viral vector, including recombinant lentivirus, recombinant adenovirus, and recombinant adenovirus associated virus. The genetic construct may be part of the genetic material in attenuated live microorganisms or recombinant microbial vectors which live in cells. The genetic constructs may comprise regulatory elements for gene expression of the coding sequences of the nucleic acid. The regulatory elements may be a promoter, an enhancer, an initiation codon, a stop codon, or a polyadenylation signal.

The nucleic acid sequences may make up a genetic construct that may be a vector. The vector may be capable of expressing the fusion protein, such as the CRISPR/Cas9-based gene activation system, in the cell of a mammal. The vector may be recombinant. The vector may comprise heterologous nucleic acid encoding the fusion protein, such as the CRISPR/Cas9-based gene activation system. The vector may be a plasmid. The vector may be useful for transfecting cells with nucleic acid encoding the CRISPR/Cas9-based gene activation system, which the transformed host cell is cultured and maintained under conditions wherein expression of the CRISPR/Cas9-based gene activation system takes place.

Coding sequences may be optimized for stability and high levels of expression. In some instances, codons are selected to reduce secondary structure formation of the RNA such as that formed due to intramolecular bonding.

The vector may comprise heterologous nucleic acid encoding the CRISPR/Cas9-based gene activation system and may further comprise an initiation codon, which may be upstream of the CRISPR/Cas9-based gene activation system coding sequence, and a stop codon, which may be downstream of the CRISPR/Cas9-based gene activation system coding sequence. The initiation and termination codon may be in frame with the CRISPR/Cas9-based gene activation system coding sequence. The vector may also comprise a promoter that is operably linked to the CRISPR/Cas9-based gene activation system coding sequence. The CRISPR/Cas9-based gene activation system may be under a light-inducible or chemically inducible control to enable the dynamic control of gene activation in space and time. The promoter operably linked to the CRISPR/Cas9-based gene activation system coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metalothionein. The promoter may also be a tissue specific promoter, such as a muscle or skin specific promoter, natural or synthetic. Examples of such promoters are described in U.S. Patent Application Publication No US20040175727, the contents of which are incorporated herein in its entirety.

The vector may also comprise a polyadenylation signal, which may be downstream of the CRISPR/Cas9-based gene activation system. The polyadenylation signal may be a SV40 polyadenylation signal. LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 vector (Invitrogen, San Diego, Calif.).

The vector may also comprise an enhancer upstream of the CRISPR/Cas9-based gene activation system, i.e., the CRISPR/Cas9-based acetyltransferase coding sequence or sgRNAs. The enhancer may be necessary for DNA expression. The enhancer may be human actin, human myosin, human hemoglobin, human muscle creatine or a viral enhancer such as one from CMV, HA, RSV or EBV. Polynucleotide function enhancers are described in U.S. Pat. Nos. 5,593,972, 5,962,428, and WO94/016737, the contents of each are fully incorporated by reference. The vector may also comprise a mammalian origin of replication in order to maintain the vector extrachromosomally and produce multiple copies of the vector in a cell. The vector may also comprise a regulatory sequence, which may be well suited for gene expression in a mammalian or human cell into which the vector is administered. The vector may also comprise a reporter gene, such as green fluorescent protein (“GFP”) and/or a selectable marker, such as hygromycin (“Hygro”).

The vector may be expression sectors or systems to produce protein by routine techniques and readily available starting materials including Sambrook et al., Molecular Cloning and Laboratory Manual, Second Ed., Cold Spring Harbor (1989), which is incorporated fully b % reference. In some embodiments the sector may comprise the nucleic acid sequence encoding the CRISPR/Cas9-based gene activation system, including the nucleic acid sequence encoding the CRISPR/Cas9-based acetyltransferase and the nucleic acid sequence encoding the at least one gRNA.

### (ii) Combinations

The CRISPR/Cas9-based gene activation system composition may be combined with orthogonal dCas9s, TALEs, and zinc finger proteins to facilitate studies of independent targeting of particular effector functions to distinct loci. In some embodiments, the CRISPR/Cas9-based gene activation system composition may be multiplexed with various activators, repressors, and epigenetic modifiers to precisely control cell phenotype or decipher complex networks of gene regulation.

### B. Methods of Delivery

Further provided herein are methods for delivering the present pharmaceutical formulations comprising the engineered lysine acyltransferase (e.g., p300 or CBP), such as a CRISPR/Cas9-based gene activation system comprising engineered p300 for providing genetic constructs and/or proteins of the CRISPR/Cas9-based gene activation system. Delivery may comprise transfection or electroporation as one or more nucleic acid molecules that is expressed in the cell and delivered to the surface of the cell. The present engineered acyltransferase (e.g., p300 or CBP), protein or fusion protein may be delivered to the cell. The nucleic acid molecules may be electroporated using BioRad Gene Pulser Xcell or Amaxa Nucleofector IIb devices or other electroporation device Several different buffers may be used, including BioRad electroporation solution, Sigma phosphate-buffered saline product #D8537 (PBS), Invitrogen OptiMEM I (OM), or Amaxa Nucleofector solution V (N.V.). Transfections may include a transfection reagent, such as Lipofectamine 2000.

The vector encoding the engineered acyltransferase (e.g., p300 or CBP), or fusion protein, such as the CRISPR/Cas9-based gene activation system protein, may be delivered to the mammal by DNA injection (also referred to as DNA vaccination) with and without in vivo electroporation, liposome mediated, nanoparticle facilitated, and/or recombinant vectors. The recombinant vector may be delivered by any viral mode. The viral mode may be recombinant lentivirus, recombinant adenovirus, and/or recombinant adeno-associated virus.

The polynucleotides of the present disclosure may be introduced (e.g., transfected or transduced) into a host cell by viral or non-viral methods. Vectors provided herein are designed, primarily, to express a suicide gene under the control of a cell-cycle dependent promoter. One of skill in the art would be well-equipped to construct a vector through standard recombinant techniques (see, for example, Sambrook et al., 2001 and Ausubel et al., 1996, both incorporated herein by reference). Vectors include but are not limited to, plasmids, cosmids, viruses (e.g., bacteriophage, animal viruses, and plant viruses), artificial chromosomes (e.g., YACs), retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, maraba virus vectors and group B adenovirus enadenotucirev vectors.

“Lentivirus” refers to a virus belonging to the lentivirus genus Lentiviruses include, but are not limited to, human immunodeficiency virus (HIV) (for example, HIV-1 or HIV-2), simian immunodeficiency virus (SIV), feline immunodeficiency virus (FIV), Maedi-Visna-like virus (EV1), equine infectious anemia virus (EIAV) and caprine arthritis encephalitis virus (CAEV).

The terms “lentiviral vector construct,” “lentiviral vector,” and “recombinant lentiviral vector” are used interchangeably herein and refer to a nucleic acid construct derived from a lentivirus that carries and, within certain embodiments, is capable of directing the expression of a nucleic acid molecule of interest. Lentiviral vectors can have one or more of the lentiviral wild-type genes deleted in whole or part but retain functional flanking long-terminal repeat (LTR) sequences. The LTRs need not be the wild-type nucleotide sequences, and may be altered, e.g., by the insertion, deletion or substitution of nucleotides, so long as the sequences provide for functional rescue, replication, and packaging. The lentiviral vector may also contain a selectable marker.

The term “recombinant lentivirus” refers to a virus particle that contains a lentivirus-derived viral genome, lacks the self-renewal ability, and has the ability to introduce a nucleic acid molecule into a host. For example, the recombinant lentiviruses of the present disclosure include virus particles comprising a nucleic acid molecule that comprises a lentiviral genome-derived packaging signal sequence. The recombinant lentivirus is capable of reverse transcribing its genetic material into DNA and incorporating this genetic material into a host cell's DNA upon infection. Recombinant lentivirus particles may have a lentiviral envelope, a non-lentiviral envelope (e.g., an amphotropic or VSV-G envelope), a chimeric envelope, or a modified envelope (e.g., truncated envelopes or envelopes containing hybrid sequences).

A nucleic acid carried by a lentiviral vector of the present disclosure can be introduced into pluripotent stem cells or neural progenitor cells by contacting this vector with pluripotent stem cells of primates, including humans, or rodents, including mice and rats. The present disclosure relates to methods for introducing suicide genes into pluripotent stem cells, which comprise the step of contacting pluripotent stem cells with the vectors of the present disclosure. The pluripotent stem cells targeted for gene introduction are not particularly limited and, for example, include embryonic stem cells or induced pluripotent stem cells.

The nucleotide encoding the engineered acyltransferase (e.g., p300 or CBP), or fusion protein, such as the CRISPR/Cas9-based gene activation system protein, may be introduced into a cell to induce gene expression of the target gene. For example, one or more nucleotide sequences encoding the engineered acyltransferase (e.g., p300 or CBP), or fusion protein, such as the CRISPR/Cas9-based gene activation system protein, directed towards a target gene may be introduced into a mammalian cell. Upon delivery of the engineered acyltransferase (e.g., p300 or CBP), or fusion protein, such as the CRISPR/Cas9-based gene activation system protein, to the cell, and thereupon the vector into the cells of the mammal, the transfected cells will express the engineered acyltransferase (e.g., p300 or CBP), or fusion protein, such as the CRISPR/Cas9-based gene activation system protein. The engineered acyltransferase (e.g., p300 or CBP), or fusion protein, such as the CRISPR/Cas9-based gene activation system protein, may be administered to a mammal to induce or modulate gene expression of the target gene in a mammal. The mammal may be human, non-human primate, cow, pig, sheep, goat, antelope, bison, water buffalo, bovids, deer, hedgehogs, elephants, llama, alpaca, mice, rats, or chicken, and preferably human, cow, pig, or chicken.

The engineered acyltransferase (e.g., p300 or CBP), or fusion protein, such as the CRISPR/Cas9-based gene activation system protein, and compositions thereof may be administered to a subject by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian may readily determine the dosing regimen and route of administration that is most appropriate for a particular animal. The engineered acyltransferase (e.g., p300 or CBP), or fusion protein, such as the CRISPR/Cas9-based gene activation system protein, and compositions thereof may be administered by traditional syringes, needleless injection devices, “microprojectile bombardment gone guns”, or other physical methods such as electroporation (“EP”), “hydrodynamic method”, or ultrasound. The composition may be delivered to the mammal by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus.

The engineered acyltransferase (e.g., p300 or CBP), or fusion protein, such as the CRISPR/Cas9-based gene activation system protein, may be used with any type of cell. In some embodiments, the cell is a bacterial cell, a fungal cell, an archaea cell, a plant cell or an animal cell. In some embodiments, the cell may be an ENCODE cell line, including but not limited to, GM12878, K562, H1 human embryonic stein cells, HeLa-S3, HepG2, HUVEC, SK-N-SH, IMR90, A549, MCF7, HMEC or LHCM, CD14+, CD20+, primary heart or liver cells, differentiated H1 cells, 8988T, Adult_CD4_naive, Adult_CD4_Th0, Adult_CD4_Th1, AG04449, AG04450, AG09309, AG09319, AG10803, AoAF, AoSMC, BC_Adipose_UHN00001, BC_Adrenal_Gland_H12803N, BC_Bladder_01-11002, BC_Brain_H11058N, BC_Breast_02-03015, BC_Colon_01-11002, BC_Colon_H12817N, BC_Esophagus_01-11002, BC_Esophagus_H12817N, BC_Jejunum_H12817N, BC_Kidney_01-11002, BC_Kidney_H12817N, BC_Left_Ventricle_N41, BC_Leukocyte_UHN00204, BC_Liver_01-11002, BC_Lung_01-11002, BC_Lung_H12817N, BC_Pancreas_H12817N, BC_Penis_H12817N, BC_Pericardium_H12529N, BC_Placenta_UHN00189, BC_Prostate_Gland_H12817N, BC_Rectum_N29, BC_Skeletal_Muscle_01-11002, BC_Skeletal_Muscle_H12817N, BC_Skin_01-11002, BC_Small_Intestine_01-11002, BC_Spleen_H12817N, BC_Stomach_01-11002, BC_Stomach_H12817N, BC_Testis_N30, BC_Uterus_BN0765, BE2_C, BG02ES, BG02ES-EBD, BJ, bone_marrow_HS27a, bone_marrow_HS5, bone_marrow_MSC, Breast_OC, Caco-2, CD20+_RO01778, CD20+_RO01794, CD34+_Mobilized, CD4+_Naive_Wb11970640, CD4+_Naive_Wb78495824, Cerebellum_OC, Cerebrum_frontal_OC, Chorion, CLL, CMK, Colo829, Colon_BC, Colon_OC, Cord_CD4_naive, Cord_CD4_Th0, Cord_CD4_Th1, Decidua, Dnd41, ECC-1, Endometrium_OC, Esophagus_BC, Fibrobl, Fibrobl_GM03348, FibroP, FibroP_AG08395, FibroP_AG08396, FibroP_AG20443, Frontal_cortex_OC, GC_B_cell, Gliobla, GM04503, GM04504, GM06990, GM08714, GM10248, GM10266, GM10847, GM12801, GM12812, GM12813, GM12864, GM12865, GM12866, GM12867, GM12868, GM12869, GM12870, GM12871, GM12872, GM12873, GM12874, GM12875, GM12878-XiMat, GM12891, GM12892, GM13976, GM13977, GM15510, GM18505, GM18507, GM18526, GM18951, GM19099, GM19193, GM19238, GM19239, GM19240, GM20000, H0287, H1-neurons, H7-hESC, H9ES, H9ES-AFP−, H9ES-AFP+, H9ES-CM, H9ES-E, H9ES-EB, H9ES-EBD, HAc, HAEpiC, HA-h, HAL, HAoAF, HAoAF_6090101.11, HAoAF_61113019, HAoEC, HAoEC_7071706.1, HAoEC_8061102.1, HA-sp, HBMEC, HBVP, HBVSMC, HCF, HCFaa, HCH, HCH_0011308.2P, HCH_8100808.2, HCM, HConF, HCPEpiC, HCT-116, Heart_OC, Heart_STL003, HEEpiC, HEK293, HEK293T, HEK293-T-REx, Hepatocytes, HFDPC, HFDPC_0100503.2, HFDPC_0102703.3, HFF, HFF-Myc, HFL11W, HFL24W, HGF, HHSEC, HIPEpiC, HL-60, HMEpC, HMEpC_6022801.3, HMF, hMNC-CB, hMNC-CB_8072802.6, hMNC-CB_9111701.6, hMNC-PB, hMNC-PB 0022330.9, hMNC-PB 0082430.9, hMSC-AT, hMSC-AT 0102604.12, hMSC-AT 9061601.12, hMSC-BM, hMSC-BM_0050602.11, hMSC-BM_0051105.11, hMSC-UC, hMSC-UC_0052501.7, hMSC-UC_0081101.7, HMVEC-dAd, HMVEC-dBl-Ad, HMVEC-dBl-Neo, HMVEC-dLy-Ad, HMVEC-dLy-Neo, HMVEC-dNeo, HMVEC-LB1, HMVEC-LLy, HNPCEpiC, HOB, HOB_0090202.1, HOB_0091301, HPAEC, HPAEpiC, HPAF, HPC-PL, HPC-PL_0032601.13, HPC-PL_0101504.13, HPDE6-E6E7, HPdLF, HPF, HPIEpC, HPIEpC_9012801.2, HPIEpC_9041503.2, HRCEpiC, HRE, HRGEC, HRPEpiC, HSaVEC, HSaVEC_0022202.16, HSaVEC_9100101.15, HSMM, HSMM_emb, HSMM_FSHD, HSMMtube, HSMMtube_emb, HSMMtube_FSHD, HT-1080, HTR8svn, Huh-7, Huh-7.5, HVMF, HVMF_6091203.3, HVMF_6100401.3, HWP, HWP_0092205, HWP8120201.5, iPS, iPS CWRU1, iPS_hFib2_iPS4, iPS_hFib2_iPS5, iPS_NIHi11, iPS_NIHi7, Ishikawa, Jurkat, Kidney_BC, Kidney_OC, LHCN-M2, LHSR, Liver_OC, Liver_STL004, Liver_STLO11, LNCaP, Loucy, Lung BC, Lung_OC, Lymphoblastoid_cell_line, M059J, MCF10A-Er-Src, MCF-7, MDA-MB-231, Medullo, Medullo_D341, Mel_2183, Melano, Monocytes-CD14+, Monocytes-CD 14+_RO01746, Monocytes-CD14+_RO01826, MRT_A204, MRT_G401, MRT_TTC549, Myometr, Naive_B_cell, NB4, NH-A, NHBE, NHBE_RA, NHDF, NHDF_0060801.3, NHDF_7071701.2, NHDF-Ad, NHDF-neo, NHEK, NHEM.f_M2, NHEM.f_M2_5071302.2, NHEM.f_M2_6022001, NHEM_M2, NHEM_M2_7011001.2, NHEM_M2_7012303, NHLF, NT2-D1, Olf_neurosphere, Osteobl, ovcar-3, PANC-1, Pancreas_OC, PanIsletD, PanIslets, PBDE, PBDEFetal, PBMC, PFSK-1, pHTE, Pons_OC, PrEC, ProgFib, Prostate, Prostate_OC, Psoas_muscle_OC, Raji, RCC_7860, RPMI-7951, RPTEC, RWPE1, SAEC, SH-SY5Y, Skeletal_Muscle_BC, SkMC, SKMC, SkMC_8121902.17, SkMC_9011302, SK-N-MC, SK-N-SH_RA, Small_intestine_OC, Spleen_OC, Stellate, Stomach_BC, T_cells_CD4+, T-47D, T98G, TBEC, Th1, Th1_Wb33676984, Th1_Wb54553204, Th17, Th2, Th2 Wb33676984, Th2_Wb54553204, Treg_Wb78495824, Treg_Wb83319432, U2OS, U87, UCH-1, Urothelia, WERI-Rb-1, and WI-38.

### C. Methods of Use

Potential applications of the engineered lysine acyltransferase, such as p300 or p300 fusion protein, such as the CRISPR/Cas9-based gene activation system protein, are diverse across many areas of science and biotechnology. The engineered lysine acyltransferase or lysine acyltransferase fusion protein, such as the CRISPR/Cas9-based gene activation system protein, may be used to activate gene expression of a target gene or target a target enhancer or target regulatory element. The engineered lysine acyltransferase (e.g., acetyltransferase) or lysine acyltransferase (e.g., acetyltransferase) fusion protein, such as the CRISPR/Cas9-based gene activation system protein, may be used to transdifferentiate a cell and/or activate genes related to cell and gene therapy, genetic reprogramming, and regenerative medicine. The engineered lysine acyltransferase (e.g., acetyltransferase) or lysine acyltransferase (e.g., acetyltransferase) fusion protein, such as the CRISPR/Cas9-based gene activation system protein, may be used to reprogram cell lineage specification. Activation of endogenous genes encoding the key regulators of cell fate, rather than forced overexpression of these factors, may potentially lead to more rapid, efficient, stable, or specific methods for genetic reprogramming and transdifferentiation. The engineered lysine acyltransferase (e.g., acetyltransferase) or lysine acyltransferase (e.g., acetyltransferase) fusion protein, such as the CRISPR/Cas9-based gene activation system protein, could provide a greater diversity of transcriptional activators to complement other tools for modulating mammalian gene expression. The engineered lysine acyltransferase (e.g., acetyltransferase) or lysine acyltransferase (e.g., acetyltransferase) fusion protein, such as the CRISPR/Cas9-based gene activation system protein, may be used to compensate for genetic defects, suppress angiogenesis, inactivate oncogenes, activate silenced tumor suppressors, regenerate tissue or reprogram genes.

In certain embodiments, the present disclosure provides a mechanism for activating the expression of target genes based on targeting a histone acyltransferase (e.g., acetyltransferase) to a target region via a engineered lysine acyltransferase (e.g., acetyltransferase) or lysine acyltransferase (e.g., acetyltransferase) fusion protein, such as the CRISPR/Cas9-based gene activation system protein, as described above. The engineered lysine acyltransferase (e.g., acetyltransferase) or lysine acyltransferase (e.g., acetyltransferase) fusion protein, such as the CRISPR/Cas9-based gene activation system protein, may activate silenced genes. The engineered lysine acyltransferase (e.g., acetyltransferase) or lysine acyltransferase (e.g., acetyltransferase) fusion protein, such as the CRISPR/Cas9-based gene activation system protein, target regions upstream of the TSS of the target gene and substantially induced gene expression of the target gene. The polynucleotide encoding the engineered lysine acyltransferase (e.g., acetyltransferase) or lysine acyltransferase (e.g., acetyltransferase) fusion protein, such as the CRISPR/Cas9-based gene activation system protein, can also be transfected directly to cells.

The method may include administering to a cell or subject a engineered lysine acyltransferase (e.g., acetyltransferase) or lysine acyltransferase (e.g., acetyltransferase) fusion protein, such as the CRISPR/Cas9-based gene activation system protein, compositions of engineered lysine acyltransferase (e.g., acetyltransferase) or lysine acyltransferase (e.g., acetyltransferase) fusion protein, such as the CRISPR/Cas9-based gene activation system protein, or one or more polynucleotides or vectors encoding said engineered lysine acyltransferase (e.g., acetyltransferase) or lysine acyltransferase (e.g., acetyltransferase) fusion protein, such as the CRISPR/Cas9-based gene activation system protein, as described above. The method may include administering a engineered lysine acyltransferase (e.g., acetyltransferase) or lysine acyltransferase (e.g., acetyltransferase) fusion protein, such as the CRISPR/Cas9-based gene activation system protein, compositions of engineered lysine acyltransferase (e.g., acetyltransferase) or lysine acyltransferase (e.g., acetyltransferase) fusion protein, such as the CRISPR/Cas9-based gene activation system protein, or one or more polynucleotides or vectors encoding said engineered lysine acyltransferase (e.g., acetyltransferase) or lysine acyltransferase (e.g., acetyltransferase) fusion protein, such as the CRISPR/Cas9-based gene activation system protein, as described above, to a mammalian cell or subject.

Chronic liver disease (CLD) is a major health problem worldwide, with significant impact on morbidity and mortality rates. The condition is characterized by progressive inflammation, fibrosis, and liver cell damage, resulting in the loss of normal liver function. Common causes of CLD include alcohol abuse, hepatitis B and C virus infections, nonalcoholic fatty liver disease, and autoimmune liver diseases. Statistics indicate that the burden of CLD is increasing, with an estimated 1.1 million deaths attributed to the disease in 2019. Additionally. CLD is the 12th leading cause of death globally, and its incidence is higher in low- and middle-income countries. The disease can also have significant economic and social impacts, including increased healthcare costs, reduced work productivity, and impaired quality of life for affected individuals. The impact of CLD on human health is significant, with potential complications including liver cirrhosis, liver failure, and liver cancer. These complications can lead to a range of symptoms and complications, such as ascites, hepatic encephalopathy, and gastrointestinal bleeding. Furthermore, the severity of CLD can also increase the risk of other health problems, such as cardiovascular disease and diabetes. Prevention and management of CLD involves lifestyle changes, such as reducing alcohol consumption and maintaining a healthy weight, as well as medical interventions, including antiviral therapy for viral hepatitis and immunosuppressive agents for autoimmune liver disease. Early detection and treatment of CLD are crucial for improving patient outcomes and reducing the burden of the disease on individuals and society.

The present CRISPR/Cas-based epigenome editing tool could be used to regulate gene expression related to the disease Epigenome editing utilizes a DNA-binding domain fused to an epigenome-modifying domain and alters the transcriptional output of a targeted gene of interest through modulation of the chromatin environment. The present engineered versions of the endogenous human lysine acetyltransferase (KAT), p300, are conjugated with dCas9 protein to facilitate robust gene activation of endogenous genes targeting both promoters and enhancers. In one embodiment, the p300 I1417N mutant, improves cell viability, lentiviral packing efficiency and delivery compared to p300 WT. Based on this rationale, it may be targeted to the promoter of low-density lipoprotein receptor (LDLR) gene which is a well-characterized gene related to lipid accumulation in liver and demonstrated that its activity can tune expression level of causative genes of chronic liver diseases. Overall, this technology, and the methods and compositions used, are molecular tools to deposit histone acetylation to a variety of cis regulatory elements in liver and paving a new era in precision, persistence therapy of metabolic and liver disorders. The CRISPR/Cas-based epigenome editing tool described herein has significant potential in the treatment of chronic liver diseases. By regulating gene expression related to the disease from promoters and enhancers, this technology could target the underlying molecular mechanisms and potentially reverse or prevent disease progression.

### D. Formulation and Administration

The present disclosure provides pharmaceutical compositions comprising the engineered lysine acyltransferase (e.g., acetyltransferase) provided herein. The engineered lysine acyltransferase (e.g., acetyltransferase) or lysine acyltransferase (e.g., acetyltransferase) fusion protein, such as the CRISPR/Cas9-based gene activation system protein, may be in a pharmaceutical composition. The pharmaceutical composition may comprise about 1 ng to about 10 mg of DNA encoding the engineered lysine acyltransferase (e.g., acetyltransferase) or lysine acyltransferase (e.g., acetyltransferase) fusion protein, such as the CRISPR/Cas9-based gene activation system protein. The pharmaceutical compositions according to the present invention are formulated according to the mode of administration to be used. In cases where pharmaceutical compositions are injectable pharmaceutical compositions, they are sterile, pyrogen free and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The pharmaceutical composition containing the engineered lysine acyltransferase (e.g., acetyltransferase) or lysine acyltransferase (e.g., acetyltransferase) fusion protein, such as the CRISPR/Cas9-based gene activation system protein, may further comprise a pharmaceutically acceptable excipient. The pharmaceutically acceptable excipient may be functional molecules as vehicles, adjuvants, carriers, or diluents. The pharmaceutically acceptable excipient may be a transfection facilitating agent, which may include surface active agents, such as immune-stimulating complexes (ISCOMS). Freunds incomplete adjuvant. LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and more preferably, the poly-L-glutamate is present in the pharmaceutical composition containing the engineered lysine acetyltransferase or lysine acetyltransferase fusion protein, such as the CRISPR/Cas9-based gene activation system protein, at a concentration less than 6 mg/ml. The transfection facilitating agent may also include surface active agents such as immune-stimulating complexes (ISCOMS). Freunds incomplete adjuvant. LPS analog including monophosphoryl lipid A, muramyl peptides, quinone analogs and vesicles such as squalene and squalene, and hyaluronic acid may also be used administered in conjunction with the genetic construct. In some embodiments, the DNA vector encoding the engineered lysine acetyltransferase or lysine acetyltransferase fusion protein, such as the CRISPR/Cas9-based gene activation system protein, may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. Preferably, the transfection facilitating agent is a polyanion, polycation, including poly-L-glutamate (LGS), or lipid.

Such compositions comprise a prophylactically or therapeutically effective amount of an antibody or a fragment thereof, or a peptide immunogen, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, or delivered by mechanical ventilation.

Active vaccines are also envisioned where antibodies like those disclosed are produced in vivo in a subject at risk of Poxvirus infection. Such vaccines can be formulated for parenteral administration. e.g., formulated for injection via the intradermal, intravenous, intramuscular, subcutaneous, or even intraperitoneal routes. Administration by intradermal and intramuscular routes are contemplated. The vaccine could alternatively be administered by a topical route directly to the mucosa, for example by nasal drops, inhalation, or by nebulizer. Pharmaceutically acceptable salts, include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Passive transfer of antibodies, known as artificially acquired passive immunity, generally will involve the use of intravenous or intramuscular injections. The forms of antibody can be human or animal blood plasma or serum, as pooled human immunoglobulin for intravenous (IVIG) or intramuscular (IG) use, as high-titer human IVIG or IG from immunized or from donors recovering from disease, and as monoclonal antibodies (MAb). Such immunity generally lasts for only a short period of time, and there is also a potential risk for hypersensitivity reactions, and serum sickness, especially from gamma globulin of non-human origin. However, passive immunity provides immediate protection. The antibodies will be formulated in a carrier suitable for injection. i.e., sterile and syringeable.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### E. Kits

Provided herein is a kit, which may be used to activate gene expression of a target gene.

The kit comprises a composition for activating gene expression, as described above, and instructions for using said composition. Instructions included in kits may be affixed to packaging material or may be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g. CD ROM), and the like. As used herein, the term “instructions” may include the address of an internet site that provides the instructions.

The composition for activating gene expression may include a lentiviral vector and a nucleotide sequence encoding an engineered lysine acyltransferase (e.g., acetyltransferase) or lysine acyltransferase (e.g., acetyltransferase) fusion protein, such as the CRISPR/Cas9-based gene activation system protein, as described above. The engineered lysine acyltransferase (e.g., acetyltransferase) or lysine acyltransferase (e.g., acetyltransferase) fusion protein, such as the CRISPR/Cas9-based gene activation system protein, may include engineered lysine acyltransferase (e.g., acetyltransferase) or lysine acyltransferase (e.g., acetyltransferase) fusion protein, such as the CRISPR/Cas9-based gene activation system protein, as described above, that specifically binds and targets a cis-regulatory region or trans-regulatory region of a target gene. The engineered lysine acyltransferase (e.g., acetyltransferase) or lysine acyltransferase (e.g., acetyltransferase) fusion protein, such as the CRISPR/Cas9-based gene activation system protein, as described above, may be included in the kit to specifically bind and target a particular regulatory region of the target gene.

### III. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Engineering and Characterization of Lysine Acetyltransferase

An unbiased approach was taken towards optimizing the activity and stability of p300. A transcriptional reporter system was utilized wherein p300 or a single amino acid mutant variant thereof was fused to a Tet repressor protein such that it is recruited to a TetO-containing promoter driving a fluorescent reporter gene, such as Citrine, in the presence of doxycycline. This served as a proxy for acetylation as it indirectly drives gene expression within this context. The TetR-p300 fusion protein is co-translated with a different fluorescent protein (mCherry) which provides a reporter on protein expression levels. Given that protein expression is closely linked to its toxicity through currently unknown mechanisms, both p300 enzymatic activity and toxicity could be tracked through this multiparametric approach.

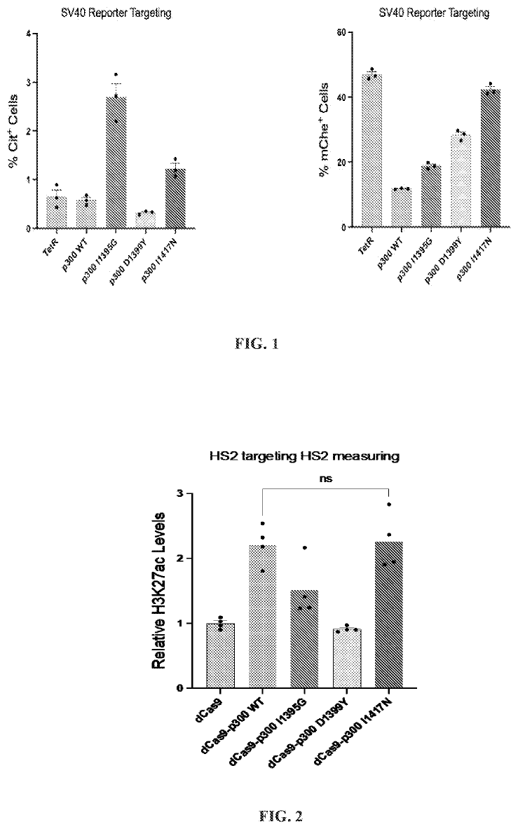

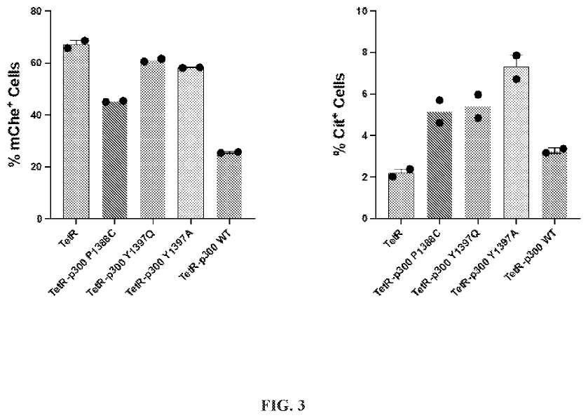

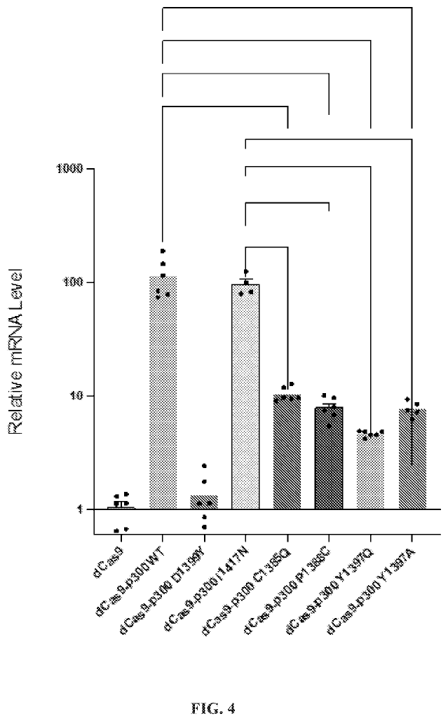

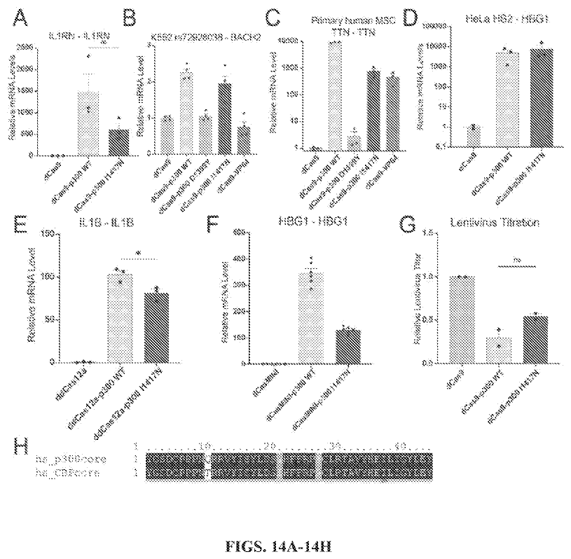

One of the hits of this screen was an I1417N mutation (hence known as p300 I1417N) that both did not affect enzymatic activity to appreciable levels and substantially improved stability (FIG. 1). In subsequent follow up studies, p300 I1417N was fused to dCas9 and targeted to various cis regulatory elements. It was demonstrated that transcription was still activated to levels comparable to p300 WT at both promoters and enhancers (FIG. 14D). Strikingly, this was done while improving cell viability to levels near dCas9 expressed alone, demonstrating the lack of cytotoxicity of this engineered protein in comparison to p300 WT which reduced cell viability and protein expression (FIG. 140).

Next, it was demonstrated that this variant of p300 enables improved packaging and delivery in lentivirus. Both the physical titer post-lentivirus packaging via qPCR and the functional titer via flow cytometry were measured and an increase in both packaging and transduction efficiency were observed, indicating this engineered p300 variant had improved delivery capabilities over the wild-type version (FIG. 14H, 20G).

Lastly, it was shown that p300 I1417N can be ported to ddCas12a, a different Cas species that is useful for multiplexing target genes due to its ability to process its own CRISPR RNAs (crRNAs). It was targeted to the IL1B promoter and demonstrated that its activity is highly similar to p300 WT within this context (FIG. 20E). Overall, the studies were able to modulate enzymatic activity (as reported on by transcriptional activation) and toxicity (as reported on by protein expression) in a manner that developed a biotechnologically useful version of p300 with minimal toxicity.

### Example 2—Tailoring a CRISPR/Cas-Based Acyltransferase for Programmable Acylation and Decreased Cytotoxicity

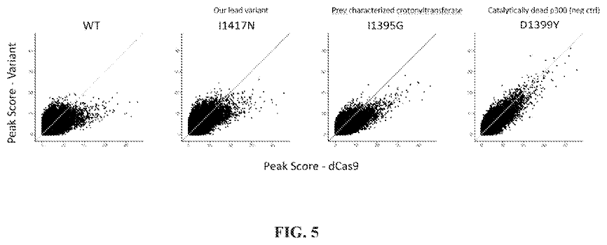

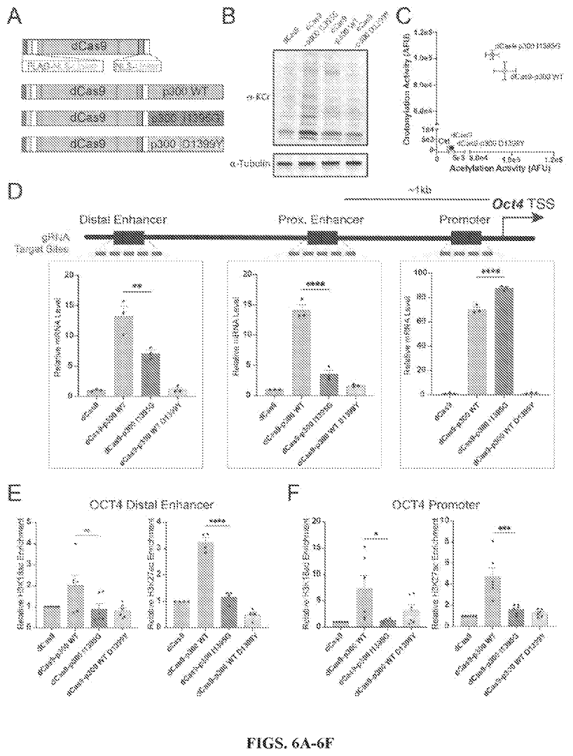

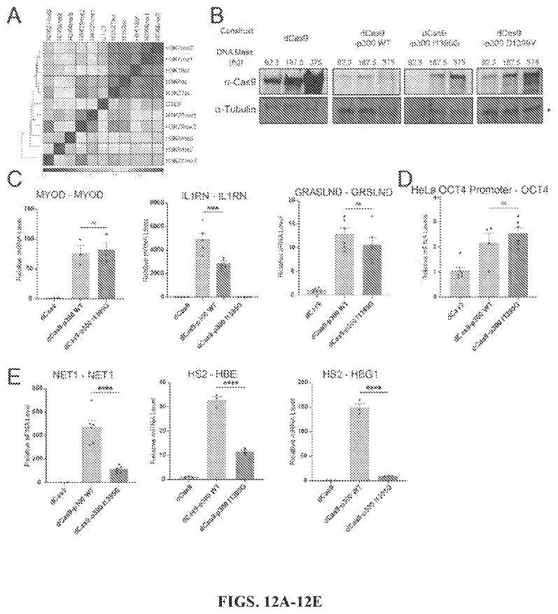

Chromatin crotonylation strongly activates transcription at promoters but not enhancers. Publicly available data was aggregated to determine the histone modifications and transcription factors that colocalize with H3 lysine 18 crotonylation (H3K18cr) in HCT116 cells. It was found that H3K18cr closely correlates with histone modifications associated with transcriptionally active loci (FIG. 12A). This finding is consistent with previous studies at both reporter genes and endogenous loci which demonstrated that exogenous addition of crotonate increases transcription (Sabari et al., 2015, Goudarzi et al., 2016). To determine how longer-chain acylations affect transcription at endogenous regulatory elements. CRISPR-based fusion proteins were constructed with different variants of the p300 core (referred to as p300 hereafter) domain, p300 was appended harboring a previously characterized mutation (p300 I1395G) that was observed to skew p300/CBP toward depositing crotonylation onto chromatin instead of acetylation (Liu et al., 2017) to the C-terminus of catalytically dead Streptococcus pyogenes Cas9 (dCas9) with (FIG. 6A). When these constructs were expressed in HEK293T cells, a marked increase of crotonylated lysine residues was observed across the proteome in comparison to the dCas9-p300 WT fusion or a catalytically inactivated control (dCas9-p300 D1399Y (Ortega et al., 2018); FIG. 6B) In addition, in vitro purified dCas9-p300 I1395G crotonylated purified histone H3 peptides to a greater degree than purified dCas9-p300 WT, with slightly decreased amounts of H3 peptide acetylation observed (FIG. 6C). These results demonstrate that the I1395G mutation can crotonylate lysine residues on histone and non-histone proteins to a greater degree than p300 WT. A range of plasmid amounts were also transfected and, interestingly, found that dCas9 fused to p300 WT was expressed at much lower levels than dCas9-p300 I1395G or dCas9-p300 D1399Y (FIG. 12B). Further, it was observed that, when expressed at high enough levels (~375ng of input DNA). dCas9-p300 WT appeared to have detrimental effects on the relative expression of the tubulin loading control, which may indicate inhibited cell growth, toxicity, or cellular senescence upon p300 overexpression (Olzscha et al., 2017; Sen et al., 2019).

Upon targeting human promoters using a pool of guide RNAs (gRNAs), it was observed that dCas9-p300 I1395G activated transcription at a level comparable to dCas9-p300 WT from promoters (FIG. 6D, 12C), which was also observed in another cell type (FIG. 12D). However, in contrast to dCas9-p300 WT, it was found that dCas9-p300 I1395G only weakly activated the transcription of downstream genes when targeted to enhancers (FIG. 6D, 12E). Interestingly, most bromodomain-containing proteins are unable to bind crotonylated histone residues (Flynn et al., 2015; Hnisz et al., 2013; Sabari et al., 2018), which may explain the weakened enhancer-mediated gene activation displayed by dCas9-p300 I1395G. To determine if dCas9-p300 I1395G retained residual acetylation capabilities at targeted loci, CUT&RUN-qPCR was performed at the promoter of targeted gene (HBG1) A drastically reduced enrichment was observed with dCas9-p300 I1395G to a level indistinguishable from dCas9 alone or an enzymatically dead p300 (dCas9-p300 D1399Y). However, as expected, enrichment of H3K18ac and H3K27ac was found when the dCas9-p300 WT fusion proteins were targeted to either the OCT4 promoter or the OCT4 distal enhancer (FIG. 6E, 6F). These data indicate that crotonylation and acetylation are functionally divergent at endogenous human enhancers, but can be redundant at human promoters with respect to transcriptional output. Furthermore, the results demonstrate that dCas9-p300 I1395G is sufficient to activate human promoters in the absence of H3K18ac and/or H3K27ac.

A high throughput screen to evaluate the p300 HAT domain activity-expression relationship. As an effector domain for epigenome editing, p300 (and its close paralog the CBP core) (Wang et al., 2022) is the only known direct writer of H3K27ac, and is highly active when the core domain is isolated (Hilton et al., 2015). However, in many cell types, dCas9-p300 WT can be poorly expressed (Wang et al., 2022; Dancy et al., 2015; Tyckoc et al., 2023). Given that the single I1395G point mutation altered both the acyltransferase activity and expression level of p300 (FIG. 12B), it was hypothesized that other mutations within the catalytic domain of the enzyme could result in enhanced expression without sacrificing epigenome editing efficacy. To test this hypothesis, an unbiased screening approach was developed enabling characterization of other mutations within the p300 core domain in high throughput. A reverse tetracycline repressor (rTetR) fusion-based approach to recruit a library of single amino acid p300 mutants to a reporter cell line consisting of 9×TetO sites upstream of an SV40 core promoter driving a Citrine fluorescent reporter stably integrated into the AAVS1 locus (FIG. 14G) (Tycko et al., 2023).

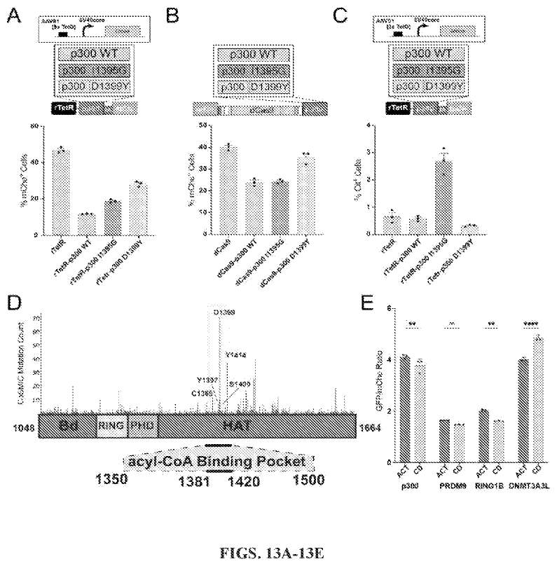

It was found that linking the rTetR to mCherry using a porcine teschovirus self-cleaving peptide (P2A) showed differing levels of expression across the characterized variants of p300, validating the rTetR system as a reporter on protein expression levels when either transduced and transiently transfected (FIG. 7B, 13A). Further, by directly fusing mCherry to the N-terminus of dCas9-p300-based fusions, it w % as confirmed that this observation is consistent at the protein level, thus linking protein expression with stability in this context (FIG. 13B). This also corroborates the Western blot data demonstrating the relative expression levels among dCas9 and dCas9-p300 variants (FIG. 12B). Collectively, these results suggest that downregulation of exogenous p300 protein is correlated with higher levels of acetylation activity.

Having established that the rTetR fusion system enabled multiparametric screening of p300 variants on both transcriptional output and relative expression, a deep mutational scanning library was designed consisting of 940 members encoding 800 point mutations, 40 single amino acid deletions, and 100 random negative controls. The library targeted the amino acids flanking a contiguous stretch of p300 (1381-1420aa) that contained the I1395G mutation and a known inactivating mutation (D1399Y) (Delvecchio et al., 2013). This region was also selected because it resides in the acyl-CoA binding pocket (~1330-1630aa) (Ortega et al., 2018), which was hypothesized might enable reshaping of the enzymatic activity of p300 based on structural analysis. This library was synthesized and inserted into the p300 core domain and then delivered resulting variants at a multiplicity of infection (MOI) of ~0.3 via lentivirus into the HEK293T cells expressing the reporter system After selection, the resulting pool of transduced cells were treated with doxycycline for 48 hours to induce recruitment of the library as described previously (Tycko et al., 2020). At day 9 post-transduction, a Sort-seq approach was used to bin the library into one of four quadrants demarcated by mChe^{hi/lo} and Cit^{hi/lo}, the resulting domains were sequenced, and HT-recruit was used to compute the log₂ (ON/OFF) ratios for each of the library members using the read counts of the sorted library populations within each of the four quadrants (Tycko et al., 2020) The measurements were highly reproducible and assigned ratios for 819 of the variants of p300.

As expected, mutations at the 1399 residue resulted in loss of transcriptional activation (FIG. 7C) which is consistent with the D1399Y catalytically dead p300 and the most prevalent mutations found in the Catalogue of Somatic Mutations in Cancer (CoSMIC) database (FIG. 13E) (Tate et al., 2019). Mutations at this residue are also linked to the neurodevelopmental disorder. Rubinstein-Taybi Syndrome (Zimmermann et al., 2007). The residues known to encompass the acyl-CoA binding site (1398-1400 and 1410-1411) appear to have a mixed effect when mutagenized in this system. For instance, only Asp1399 and Ser1400 have resoundingly disruptive effects on gene activation and this is reflected in being the predominant residues where disease-causing variants are found (FIG. 13E) (Maksimoska et al., 2014). Other residues known to contact acetyl-CoA appeared to be much more permissive of mutagenesis despite largely being hydrophobic. For example, Leu1398 and Ile1395 have been previously described to accommodate the methyl moiety of the acetyl group and are permissive of mutagenesis (FIG. 7C) (Maksimoska et al., 2014). Further, alteration at the Ile1395 imparts a greater propensity for crotonyl-CoA usage as described previously (Kaczmarska et al., 2017; Liu et al., 2017). Therefore, site-specific mutagenesis at these two residues may enable further engineering of improved longer-chain acyltransferases.

Additionally, it was found that deletions tended to disrupt transcriptional activation but had a mixed effect on protein expression. This result is concordant with the notion that loss of gene activation, and thus histone acetylation, can lead to greater expression at the protein level as was observed with p300 D1399Y (FIG. 7D). Despite the loss of transcriptional activation observed when mutagenizing the acyl-CoA binding site, it was found that these mutations had minimal effect on protein expression, suggesting that these mutations in p300 drive catalytic inactivity as opposed to changes in structure and/or misfolding. Interestingly, some frequently mutated residues found in the CoSMIC database (i.e., Cys1385 and Tyr1414; FIG. 13E) did not appear to impact the transcriptional activity of p300, but instead, increased the relative expression of p300, suggesting that p300 may be involved in oncogenesis through loss of function and/or increased expression.

Due to the demonstrable difference the catalytically dead p300 had in expression levels compared to the active enzyme, it was tested if this observation held for other types of epigenome editing enzymes. To assess this, the catalytically active or inactive domains of p300, PRDM9, RING1B, and DNMT3A3L were integrated into a stability reporter system (FIG. 7F) (Slabicki et al., 2020; Policarpi et al., 2022). It was found that both activators have reduced expression levels when active whereas the repressors that were tested are unaffected by catalytic activity. Loss of expression occurred primarily at the mRNA level (FIG. 7F, 13G), consistent with the data comparing both direct fusion and cistronic expression of p300 (FIG. 13A-C). This suggests that this mechanism of downregulation may extend to epigenome-modifying activators as a class and may be a result of transcriptional squelching or another mode of cellular toxicity driven by redistribution of stoichiometrically-controlled co-activators (Lin et al., 2007; Gillespie et al., 2020; Jones et al., 2020). Taken together with the screening regime, this data further demonstrates the utility of multiparametric tuning of expression and transcriptional activation as a powerful approach to modulate the expression and activity of these epigenome editing tools.

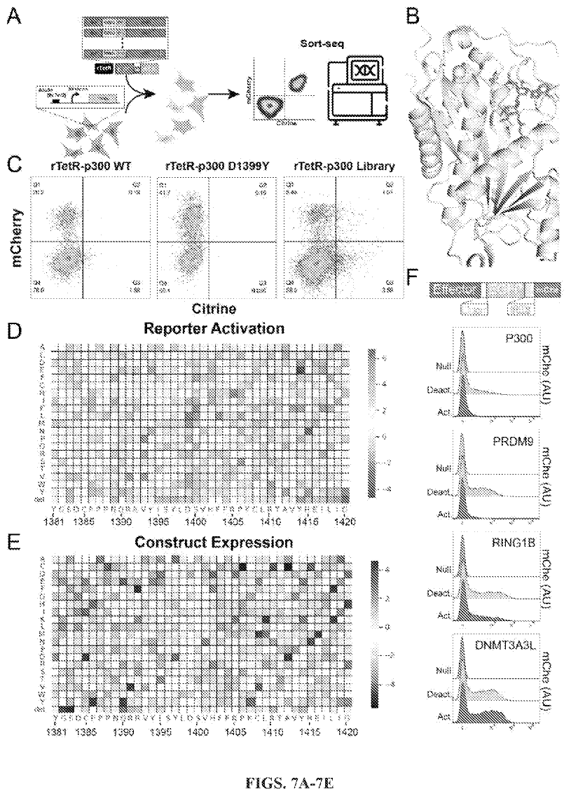

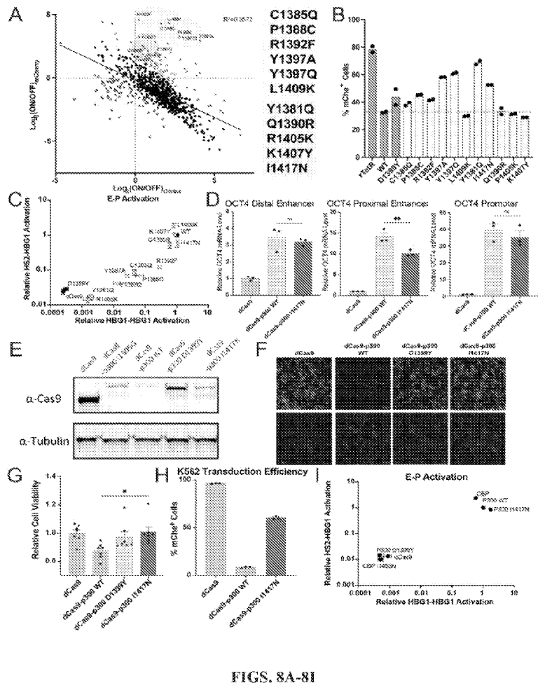

Characterization of a stable and active variant of the p300 core domain. From the deep mutational scanning of the 1381-1420aa position within the p300 core domain, two regions were identified on the activity vs, stability landscape on which validation studies were performed (FIG. 8A). Specifically, the top six scoring variants were analyzed across a combination of both protein expression and activity (mChe^{hi}Cit^{hi}). Given that p300 I1395G worked as a stronger activator than p300 WT (FIG. 6, 13A), follow-up studies were also performed on five variants with high protein expression but an intermediate level of gene activation (mChe^{hi}Cit^{mid}; FIG. 8A). It was found that 5 out of 6 of the mChe^{hi}Cit^{hi} variants and 2 out of 5 of the mChe^{hi}Cit^{mid} variants had expression levels that exceeded p300 WT (FIG. 8B). Each of these variants were next fused to dCas9 to assess relative performance at endogenous loci and found that four of the engineered variants (Q1390R, K1407Y, L1409K, and I1417N) performed comparably to p300 WT across native human promoters and enhancers (FIG. 8C). Three of these variants (Q1390R, K1407Y, I1417N) were derived from the mChe^{hi}Cit^{mid} region, supporting the rationale for enrichment of variants that activate genes similarly to p300 WT but also retained expression. Integrating these results together, the I1417N mutation was identified as a promising lead to characterize further given its expression level similar to the catalytically dead D1399Y enzyme (FIG. 8B) and enhancer and promoter activation similar to p300 WT.

It was validated that p300 I1417N activated transcription comparably to slightly less than the WT enzyme in a locus-dependent manner by targeting the regulatory elements controlling OCT4 expression as a testbed (FIGS. 8D, 14A). Further, it was confirmed that p300 I1417N displayed similar efficacy to p300 WT in other cell types (FIGS. 8B-D) and across different Cas species (ddCas12a and dCasMINI; FIGS. 14E, 14F) (Campa et al., 2019; Xu et al., 2021). It was also confirmed that protein expression correlated to the mCherry output of the polycistronic P2A system via Western blot, validating that dCas9-p300 I1417N is expressed much more highly than dCas9-p300 WT in HEK293T cells (FIG. 8E, 8F). To determine whether this improved expression level also resulted in improved cell viability upon transient transfection, a cell viability assay was performed across the different variants of p300. Strikingly, cells transfected with p300 WT displayed reduced viability compared to the p300 I1417N enzyme whereas the viability of cells expressing the engineered p300 I1417N variant was comparable to that of dCas9 alone (FIG. 8G).

Constraints on stability and cell viability are potential bottlenecks in packaging virus for downstream cell engineering (Maunder et al., 2017) and it was hypothesized that the improvement in both protein stability and cell viability would enable improved packaging of dCas9-p30 I1417N relative to dCas9-p300 WT in viral-based systems. To test this hypothesis, dCas9, dCas9-p300 WT, and dCas9-p300 I1417N were packaged in lentiviral vectors and their physical titers quantified. It was observed that dCas9-p300 I1417N had a 2-fold greater titer in these packaging experiments, thus enabling more efficient p300-based epigenome editor delivery via lentivirus (FIG. 14G). Lentiviral transduction efficiency was also quantified in K562s transduced at a defined quantity (2% v/v) and observed a greater than 5-fold improvement in transduction efficiency as measured by a co-translated mCherry fluorophore (FIG. 8H).

Next, a similar mutation was created in CBP (I1453N) as the HAT core domains are highly conserved between the two proteins (Dancy & Cole, 2015; Delvecchio et al., 2013). Upon targeting CBP WT and I1453N variants to the HBG1 and HS2 loci, it discovered that CBP I1453N lacks the ability to activate transcription at both promoters and enhancers (FIG. 8I). While this was surprising as p300 and CBP are structurally similar and are thought to be functionally redundant in many cases, previous studies have found identical mutations in CBP/p300 that lead to only minor reductions in acetylation activity for one and abolished activity in the other, pointing to subtle structural differences between the two proteins (FIG. 14H) (Dancy & Cole, 2015; Bordoli et al., 2001).

Discovery of protein-protein interactions driven by different variants of the p300 core domain. Due to the pronounced differences that p300 variants displayed relative to p300 WT in both transcriptional activation and expression levels, it was suspected that embedded mutations were altering protein-protein interactions. To investigate this possibility, immunoprecipitation was performed followed by mass spectrometry (IP-MS) on HEK293T cells transiently transfected with selected mutants of dCas9-p300 and dCas9-p300 WT (FIG. 15A). Hits were filtered out that were highly prevalent in the CRAPome (>50% occurrence) to remove false positive interactors that are common in IP-MS experiments (Mellacheruvu et al., 2013). It was found that dCas9-p300 WT exhibited the most significant differential interactors (pᵥₐₗ<0.05; compared to a dCas9-only “bait”) with 236 hits, whereas the catalytically inactivated dCas9-p300 D1399Y mutant exhibited the fewest with 115 significant hits (pᵥₐₗ<0.05; compared to a dCas9-only bait, FIG. 15A). The crotonylation depositing dCas9-p300 I1395G variant and the improved stability dCas9-p300 I1417N variant had comparable interaction hits with 169 and 163, respectively. When compared against dCas9 as a negative control bait sample, and hierarchically clustered, patterns emerged that indicated which interactions arose as a function of different activities of p300 (FIG. 15B). For example, it was observed that scaffolding-dependent interaction partners such as RNASEH2A, PTPN11, CHMP5, PREB, and CDK4 were conserved across all four variants of p300. Another hierarchical cluster contained interactors that were specific to certain acyl modifications such as MINK1 and CDK13. This cluster also contained interactions that required the acylation activity of p300 such as YTHDF1, BAG2, and SIRT2, the last of which is a histone deacetylase and known negative regulator of p300, demonstrating the close interplay between proteins that counterbalance epigenetic mechanisms, particularly histone acetylation/deacetylation activity (Black et al., 2008). Interestingly, many hits in the dCas9-p300 WT condition were unique across all variants (159/458, 34.7%) and, of the unique hits, were significantly depleted compared to the control (141/159, 88.6%; FIGS. 9C, 15B), suggesting that the acetylation activity of p300 drives a greater subset of p30 interactors as opposed to its scaffolding function and may even repel potential interacting partners.

Within the proteomics dataset, several known p300 interactions were found that are noted in the STRING database such as TAF6, WDR5, SUPT16H, and SIRT2 (FIG. 15C). Interestingly, TAF6, WDR5, and SUPT16H are depleted hits in the dataset, highlighting the function the flanking domains of the p300 core that serve as scaffolding and stabilize interactions with transcription factors and co-activators. As expected, many of the known hits are positive regulators of transcription and are only dCas9-p300 WT interactors, whereas SIRT2 interacts with all variants with acyltransferase activity (FIG. 15C). The proteomics dataset was also compared against another dataset of known acetylation targets of p300 (Weinert et al., 2018). Despite retaining the capacity to acetylate histones, dCas9-p300 I1417N had only 45% of the hits dCas9-p300 WT did and shared 60% of its hits with dCas9-p300 D1399Y, which suggests that dCas9-p300 I1417N may differ in activity from VT due to its lack of non-histone protein acetylation (FIG. 15D). To further elucidate the differences between dCas9-p300 WT and dCas9-p300 I1417N, the fold change was computed in interactions between these two variants. The stable I1417N variant interacted with a greater number of proteins at a significant level reinforcing the notion that WT-level acetylation leads to a repulsion of interactor proteins (FIG. 9C). Interestingly, some of these proteins such as DAXX, ASF1A, and UBE2L3 are involved in apoptosis and protein degradation pathways (Huang et al., 2021; Eldridge et al., 2017; Wu et al., 2019). Gene ontology analysis was performed and it was discovered that many of the dCas9-p300 I1417N hits are implicated in transcription-coupled nucleotide-excision repair and other DNA repair pathways (FIG. 9D), which is intriguing as DNA damage response proteins have been observed to be acetylated by p300 and knockdown of p300 was recently found to decrease prime editing efficiency (Dutto et al., 2018; Li et al., 2023).

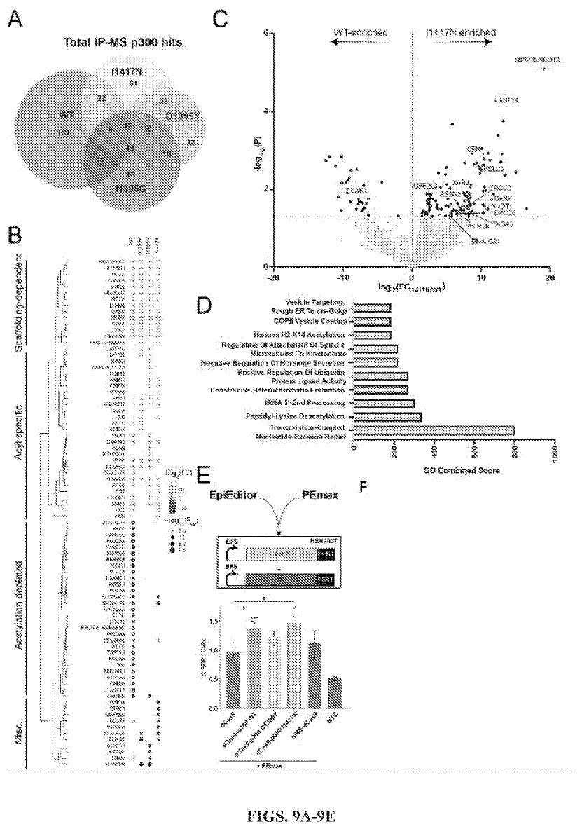

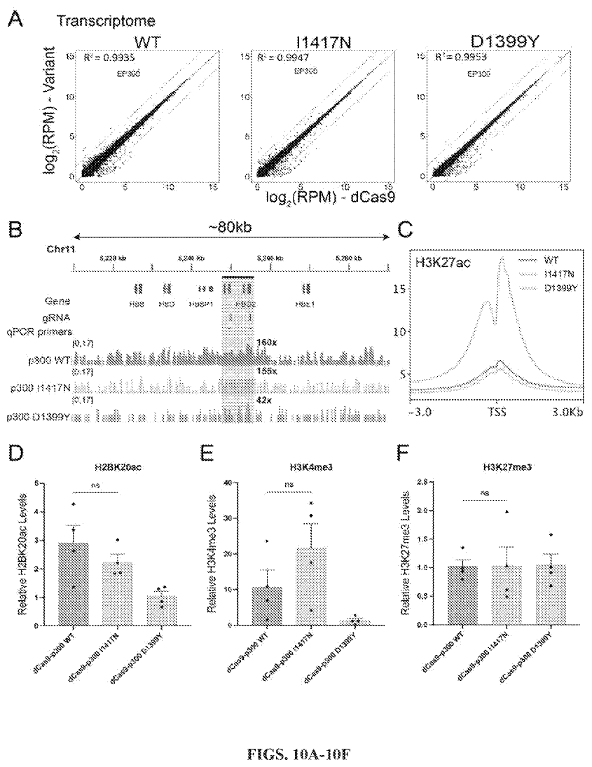

Off-target characterization of p300-mediated epigenome editing. RNA-seq was next performed to quantify how overexpression of different p300 variants globally affected the human transcriptome. Overall, it was found that transcriptional profiles were fairly consistent among these p300 variants, with minimal off-targeting compared to dCas9. A pattern was observed wherein dCas-p300 WT displayed the most off-targeting (R²=0.9935) followed by dCas9-p300 I1417N (R²=0.9947) and then dCas9-p300 D1399Y (R²=0.9953) when compared against cells expressing dCas9 alone (FIG. 10A). Most of the differentially expressed genes occurred at lowly expressed loci (FIG. 16A). Interestingly, these data are consistent with a model in which the enzymatic activity of high levels of p300 can lead to aberrant H3K27ac enrichment at enhancers that is consequently responsible for relatively small (or undetectable) changes in gene expression (Gasperini et al., 2019; Zhang et al., 2020).

To better understand both the on- and off-target profiles of dCas9-p300 WT. I1417N, and D1399Y variants at the epigenomic level. CUT&RUN was used to probe for H3K27ac in HEK293T cells in which these dCas9-based fusion proteins were targeted to the HBG1 promoter. Expectedly, high levels of on-target H3K27ac deposition were observed via dCas9-p300 WT and I1417N but not the catalytically inactive D1399Y (FIGS. 10B, 16B). Enrichment of H3K27ac was next compared across all transcription start sites (TSSs) in the genome-wide CUT&RUN datasets to characterize the off-target profile of these respective epigenome editors. Counterintuitively, it was found that both p300 WT and p300 I1417N lead to substantially reduced H3K27ac levels at TSSs compared to p300 D1399Y (FIG. 10C) which was also observed upstream of the TSS and into gene bodies (FIG. 16C). Tis relative reduction in global H3K27ac levels may be due to acetylation of other histone residues and/or transcription factors resulting in shunting of the available acetyl-CoA pool away from H3K27ac genome-wide (Shvedunova & Akhtar, 2022; Bulusu et al., 2017; Soaita et al., 2023).

Additionally, CUT&RUN-qPCR was performed across several other histone modifications to assess the epigenomic status at the HBG1 target locus. It was that found H2BK20ac, a p300/CBP-specific histone modification that is thought to designate active enhancers (Narita et al., 2023), was deposited at similar levels by both active enzymes (FIG. 10D). H3K4me3 was also probed for, as this modification has been previously found to have substantial crosstalk with H3K27ac (Jain et al., 2023; Zhao et al., 2021; Mahata et al., 2023) and indeed observed high levels of co-enrichment among these modifications (FIG. 10E). Lastly, H3K27me3 was quantified as a negative control and no significant deposition of this heterochromatin-associated histone modification was found at targeted sites (FIG. 10F). These data support the notion that p300 WT and I1417N are highly similar in terms of their capacity to acetylate histones and activate transcription while relatively divergent with respect to protein expression and effects on cell viability. The collective data indicate that dCas9-p300 I1417N has a reduced transcriptomic off-targeting profile yet retains comparable enzymatic activity in human cells in comparison to dCas9-p300 WT. Further, the data indicates that widespread H3K27ac perturbation does not necessarily lead to dramatic transcriptomic alterations, which indicates that H3K27ac can be dispensable for enhancer activity and may not be strictly necessary for transcriptional activity at promoters (Zhang et al., 2020; Martin et al., 2021; Millán-Zambrano et al., 2022).

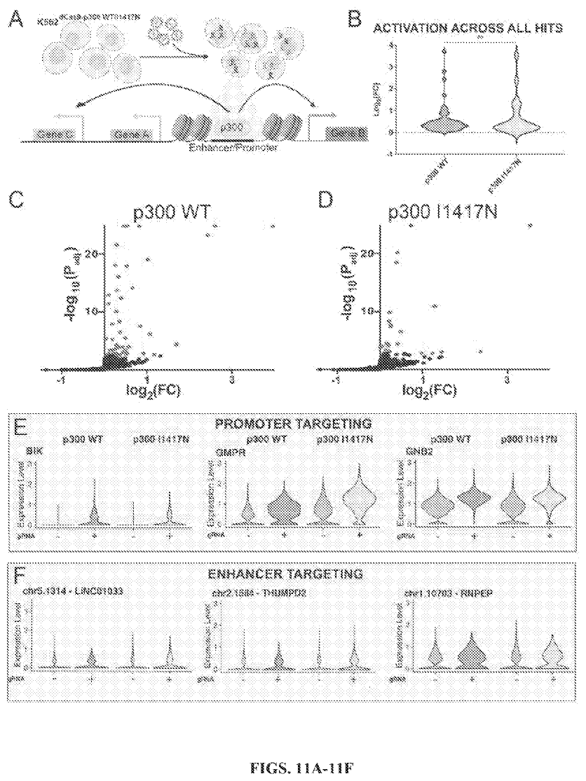

Benchmarking of p300-based Perturb-seq. It was next sought to assess both p300 WT and p300 I1417N as a tool for functional genomics profiling (Fulco et al., 2016; Yao et al., 2022; Chardon et al., 2023), monoclonal K562 cell lines expressing either dCas9-p300 WT or I1417N were derived and transgene expression levels and transactivation efficacy were validated (FIGS. 17A and 17B). A sgRNA library was generated to target candidate promoters and enhancers sourced from a previous study demonstrating proof of concept of single-cell CRISPRa (Chardon et al., 2023). A similar experimental framework was used to integrate sgRNA libraries at high MOI, performing a vector copy number titration to ensure delivery of MOI>10 library members per cell (FIG. 17C). Through scRNA-seq coupled with capture of the sgRNA, cells containing subsets of the library can be partitioned to a set containing a specific sgRNA and the complement set, linking the transcriptional profile of that group in the process as previously described (Gasperini et al., 2019; Chardon et al., 2023; Morris et al., 2023) (FIG. 11A).

K562 cells were transduced at 1% v/v lentivirus and selected for 10 days before harvesting for scRNA-seq and guide sequence capture. After quality control, 9,660 and 11,073 single-cell transcriptomes were captured with a median of 19 and 20 guides captured per cell with 392 and 484 cells assigned per gRNA for the p300 WT and p300 I1417N expressing lines, respectively (FIG. 11D, E). To perform differential expression testing, a conditional resampling approach (SCEPTRE) was used that was previously developed for improved calibration on single-cell CRISPR datasets to link perturbations with gene expression changes (Barry et al., 2021). Using the SCEPTRE pipeline, gRNAs were grouped together targeting each individual genomic element and pairwise tests performed against each gene located within 1 Mb of each gRNA location for gene expression perturbation. Good calibration was observed for the controls with non-targeting gRNAs having no effect (FIG. 17F).

Of the 434 human genome-targeting gRNAs used, 88 unique elements were targeted between promoters (38 unique elements) and enhancers (50 unique elements) previously identified and validated using CRISPRi screens. Across all hits found, the mean activation was similar between p300 WT and p300 I1417N (FIG. 11B). 48 and 24 gRNA group-gene response hits were identified for p300 WT and p300 I1417N, respectively, with 17/24 hits found in the p300 I1417N dataset shared by that of p300 WT (FIGS. 11C, 11D, 17H). While p300 WT had twice the number of statistically significant hits using the SCEPTRE framework, the average activation from called hits were comparable. Indeed, at individual promoters such as BIK, GMPR, and GNB2 comparable gene activation was observed between p300 WT and p300 I1417N (FIG. 1E). Similar trends are observed at enhancers regulating LINC01033 (chr5.1314), THUMPD2 (chr2.1584), and RNPEP (chr1.10703) wherein only LINC01033 (chr5.1314) is called a significant hit by the SCEPTRE pipeline (FIG. 11F). This was interpreted to indicate that the stringency of the SCEPTRE pipeline may lead to false negatives that would otherwise be called hits by other processing pipelines or increased cell depth. This suggests that the slightly p300 I1417N activity may cause the assay system that were used to no longer call these hits as significant. Thus, a weaker activator may cause small changes in gene expression to be lost in a scRNA-seq-based readout and is analysis method-dependent.

Because p300 functions through a different mechanism than transactivation domains, results were also compared against a previously performed scCRISPRa study that used VP64 or VPR⁶⁹. Several promoters were found that are responsive to p300 WT-mediated upregulation but was not activated by the transactivation domains VP64 or VPR (ANO5, IRS1, SCN2A, EIFIAX, and others). Conversely, only FOXP1 was found in the transactivation domain dataset but was not found as a hit with p300 WT. Enhancers were also found that activate downstream genes by p300 WT but not by transactivation domains (PCTP, LINC01993, LGALS3BP, ZNF180, THUMPD2, LINC01033, SNX18, HIST1H4H, HIST1H2BO, SLC35B2). Interestingly, only one enhancer was found that was activated by VP64/VPR but not p300 (TMEM56). Altogether, 19 enhancer-promoter pairs were found by targeting p300 WT whereas the previous dataset only uncovered 8 pairs with transactivation domains. Of these 8 pairs, 7 were shared by p300 WT, highlighting the unique capacity of p300 to activate genes from enhancers (FIG. 17G).

The present studies comprehensively studied the human p300 core domain's ability to acylate chromatin in situ, developing approaches to manipulate its function and reduce its toxicity for epigenome editing. Through single point mutations in the enzyme's binding pocket, a variant of p300 was skewed towards crotonyltransferase activity and it was established that this variant can activate promoters comparably to an acetylation-competent version of the p300 core. Conversely, the data suggest that histone crotonylation may not be sufficient to activate genes from enhancers. This is in line with recent reports pointing to YEATS-domain containing GAS41 binding to H3K27cr repressing gene expression at certain genes (Liu et al., 2023). While low levels of transcriptional activation were observed at enhancers marked with engineered crotonylation, this may be due to residual acetylation activity of p300 I1395G or competition by transcription-activating YEATS domain-containing proteins such as ENL or AF9 (Schulze et al, 2009). Given the significant role BRD4 and other bromodomain-containing proteins have in active enhancers coupled with the inability of this family of domain to bind crotonylated residues, histone crotonylation may drive weaker enhancer-mediated transcriptional regulation in tissues where crotonylation is more prevalent (Nitsch et al., 2021; Fellows et al., 2018).

As an effector domain for epigenome editing, the p300 core and its close paralog CBP are the only known writers of H3K27ac, a modification that demarcates active promoters and enhancers (Dancy & Cole, 2015). The ability to write this PTM is thus critical for understanding its role in enhancer activation and for mapping enhancers to the genes they regulate (Gasperini et al., 2020). Given its promiscuity to acylate other targets beyond H3K27ac in an “acetyl spray” mechanism, p300 has been associated with widespread off-targeting in previous studies (Weinert et al., 2018; Dominguez et al., 2022). To address this challenge, an unbiased screening approach was developed to characterize other mutations within the p300 core domain in a high throughput manner to identify high expressing p300 variants that retain the ability to activate genes from both promoters and enhancers. The I1417N mutation was validated as a novel mutation that improves stability while retaining p300's ability to activate gene expression.

In addition, improvements in p300 I1417N protein expression were linked to a reduction in genome-wide off-targeting and downstream transcriptomic changes. Interestingly, the I1417N point mutation also enabled higher efficiency packaging into lentivirus and transduction. This suggests that systematic epigenetic dysregulation may be driving minor transcriptomic alterations as well as functional changes in cells in which epigenome editors are expressed.

The protein-protein interaction profiling performed also provides an important resource in understanding the interactome of the p300 core domain and how its specific enzymatic activity regulates its engagement with other proteins. By performing IP-MS across p300 core variants with differing catalytic activities, the inventors were able to dissect which interactions were driven by the scaffolding function of p300 compared to acetylation and crotonylation. It is important to note that here the p300 core domain was used to interrogate direct PPIs associated with the catalytic activity of p300 variants. This is opposed to PPIs associated with the extensive intrinsically disordered domain-rich scaffolding that forms the flanking regions of the core domain, which are known to interact with transcription factors and other chromatin regulators (Dancy & Cole, 2015). By using the isolated core domain, this dataset is a more accurate representation of histone acetylation interactors as opposed to the complex self-regulatory and hub-like function that the full-length protein plays.

Recently, several studies have utilized CRISPRi and CRISPRa to perturb the function of the non-coding genome and thereby link enhancers to the genes that they regulate. Largely, these studies have been conducted using dCas9-KRAB and derivatives thereof (Gasperini et al., 2019; Morris et al., 2023), which provide a lens towards active enhancers and their cognate genes. However, the toolbox to perturb latent or silent enhancers remains underdeveloped, with only one other study performed to date developing CRISPRa at non-coding regions for Perturb-seq (Chardon et al., 2023). The present studies extend this toolbox to include both p300 WT and p300 I1417N. While the engineered p300 I1417N only encompasses ~50% of the hits of p300 WT using an existing Perturb-seq statistical framework, its improved transduction efficiency, decreased off-target effects, and reduced toxicity provide benefits when performing functional genomics studies on non-cancerous and primary cells.

CRISPR-based technologies that are reliant upon the recruitment of other effector domains such as base editing, prime editing, gene writing, and epigenome editing, must contend with CRISPR-independent off-target effects. The data suggests that certain domains, particularly enzymatic modifiers, may impose toxicity which must be addressed prior to translation into clinically relevant cells and in vivo usage. Therefore, comprehensive studies such as those performed here using the p300 core domain are necessary to investigate toxicities that occur beyond the established practices of studying DNA disruption.

### Example 3—Materials and Methods

Cell culture. All experiments were performed within 20 passages of cell stock thaws. HEK293T (ATCC, CRL-11268), HeLa (ATCC, CCL-2), U2OS (ATCC, HTB-96), and K562 (ATCC, CRL-243) cells were purchased from American Type Cell Culture (ATCC, USA) and cultured in ATCC-recommended media supplemented with 10% FBS (Sigma-Aldrich) and 1% pen/strep (100 units/mL penicillin, 100 μg/mL streptomycin; Gibco) at 37° C. and 5% CO2.

Plasmid construction. For spdCas9 encoding vectors (Addgene #52961 or 180293), cloning backbones were modified to have C-terminal entry sites for insertion of different effector domains. The p300 core domain was amplified from pLV-dCas9-p300-P2A-PuroR (Addgene #83889). Mutations within p300 were installed by PCR amplifying fragments of p300 with the variants encoded within and assembled into backbones via NEBuilder HiFi DNA Assembly (NEB, E2621). Similar strategies were employed for other backbones if C-terminal entry sites did not already exist. This was performed for ddCas12a (Addgene #128136), dCasMINI (Addgene #176269), and lenti pEF-rTetR(SE-G72P)-3×FLAG-LibCloneSite-T2A-mCherry-BSD-WPRE (Addgene #161926). To generate the entry vector for library cloning, p300 was shuttled into an intermediate vector (Addgene #79770), linearized by PCR at the site of interest, and an oligo encoding Esp3I sites was inserted in NEBuilder HiFi DNA Assembly. To generate the reporter plasmid with an SV40 core promoter, subcloning was performed to insert a gBlock (IDT) in the parental reporter vector, AAVS1-PuroR-9×Tet0-minCMV-IGKleader-higG1_FC-Myc-PDGFRb-T2A-Citrine-PolyA (Addgene #161928). Protein sequences of all dCas9 constructs are provided herein as SEQ ID Nos:1-9.

gRNA cloning. Oligonucleotides encoding the protospacer sequence were designed using the Design CRISPR Guides tool on Benchling, ordered from IDT, and cloned as described previously (Mahata et al., 2023). Briefly, gRNA cloning backbone (spdCas9—Addgene #47108, ddCas12a—Addgene #128136, dCasMINI—Addgene #180280, spdCas9 for scCRISPRa experiment—Addgene #192506) was digested with corresponding enzymes (Esp3I, SapI, and Esp3I, respectively). Oligos were annealed, phosphorylated, and ligated into the cloning vector using T4 Ligase (NEB). All gRNA protospacer sequences used in this study for spdCas9, dCas12a, and dCasMINI are listed in Table 4.

TABLE 4
gRNA protospacer sequences.

| | Protospacer | SEQ | Genomic Location | |
|---|---|---|---|---|
| Target | Sequence | ID | (GRCh38/hg38 | |
| Location | (5′-3′) | NO: | Assembly) | Reference |
| Oct4_PE_A | ACTTCAGGTTCAA | 14 | chr6: 31,171,948- | Hilton et al, Nat. |
| | AGAAGCC | | 31,171,967 | Biotech. 2015 |
| Oct4_PE_B | CCCTGGGTGGGG | 15 | chr6: 31,171,898- | Hilton et al, Nat. |
| | AAAACCAG | | 31,171,917 | Biotech. 2015 |
| Oct4_PE_C | TTTTCCCCACCCA | 16 | chr6: 31,171,894- | Hilton et al, Nat. |
| | GGGCCTA | | 31,171,913 | Biotech. 2015 |
| Oct4_PE_D | AGGGAGAACGGG | 17 | chr6: 31,171,843- | Hilton et al, Nat. |
| | GCCTACCG | | 31,171,862 | Biotech. 2015 |
| Oct4_PE_E | CAGACATCTAATA | 18 | chr6: 31,171,827- | Hilton et al, Nat. |
| | CCACGGT | | 31,171,846 | Biotech. 2015 |
| Oct4_PE_F | AGTGATAAGACA | 19 | chr6: 31,171,747- | Hilton et al, Nat. |
| | CCCGCTTT | | 31,171,766 | Biotech. 2015 |
| Oct4_DE_A | GCATGACAAAGG | 20 | chr6: 31,173,098- | Hilton et al, Nat. |
| | TGCCGTGA | | 31,173,117 | Biotech. 2015 |
| Oct4_DE_B | GTGCCGTGATGGT | 21 | chr6: 31,173,087- | Hilton et al, Nat. |
| | TCTGTCC | | 31,173,106 | Biotech. 2015 |
| Oct4_DE_C | GGAGGAACATGC | 22 | chr6: 31,173,032- | Hilton et al, Nat. |
| | TTCGGAAC | | 31,173,051 | Biotech. 2015 |
| Oct4_DE_D | CCTGCCTTTTGGG | 23 | chr6: 31,172,987- | Hilton et al, Nat. |
| | CAGTTAA | | 31,173,006 | Biotech. 2015 |
| Oct4_DE_E | TCGGCCTTTAACT | 24 | chr6: 31,172,980- | Hilton et al, Nat. |
| | GCCCAAA | | 31,172,999 | Biotech. 2015 |
| Oct4_DE_F | GGTCTGCCGGAA | 25 | chr6: 31,172,930- | Hilton et al, Nat. |
| | GGTCTACA | | 31,172,949 | Biotech. 2015 |
| ILIRN_ | TGTACTCTCTGAG | 26 | chr2: 113,117,865- | Perez-Pinera et |
| Promoter-1 | GTGCTC | | 113,117,883 | al., Nat. |
| | | | | Methods, 2013 |
| ILIRN_ | ACGCAGATAAGA | 27 | chr2: 113,117,714- | Perez-Pinera et |
| Promoter-2 | ACCAGTT | | 113,117,732 | al., Nat. |
| | | | | Methods, 2013 |
| ILIRN_ | CATCAAGTCAGCC | 28 | chr2: 113,117,781- | Perez-Pinera et |
| Promoter-3 | ATCAGC | | 113,117,799 | al., Nat. |
| | | | | Methods, 2013 |
| ILIRN_ | GAGTCACCCTCCT | 29 | chr2: 113,117,749- | Perez-Pinera et |
| Promoter-4 | GGAAAC | | 113,117,767 | al., Nat. |
| | | | | Methods, 2013 |
| HBG1/2_ | GCTAGGGATGAA | 30 | chr11: 5,254,807- | Perez-Pinera et |
| Promoter-1 | GAATAAA | | 5,254,825 | al., Nat. |
| | | | | Methods, 2013 |
| HBG1/2_ | TTGACCAATAGCC | 31 | chr11: 5,254,882- | Perez-Pinera et |
| Promoter-2 | TTGACA | | 5,254,901 | al., Nat. |
| | | | | Methods, 2013 |
| HBG1/2_ | TGCAAATATCTGT | 32 | chr1 1: 5,254,944- | Perez-Pinera et |
| Promoter-3 | CTGAAA | | 5,254,963 | al., Nat. |
| | | | | Methods, 2013 |
| HBG1/2_ | AAATTAGCAGTAT | 33 | chr11: 5,250,066- | Perez-Pinera et |
| Promoter-4 | CCTCTT | | 5,250,085 | al., Nat. |
| | | | | Methods, 2013 |
| HS2_Enhancer | AATATGTCACATT | 34 | chr11: 5,280,570- | Hilton et al., Nat |
| -1 | CTGTCTC | | 5,280,589 | Biotech, 2015 |
| HS2 | GGACTATGGGAG | 35 | chr11: 5,280,878- | Hilton et al., Nat |
| Enhancer-2 | GTCACTAA | | 5,280,897 | Biotech, 2015 |
| HS2 | GAAGGTTACACA | 36 | chr11: 5,280,803- | Hilton et al., Nat |
| Enhancer-3 | GAACCAGA | | 5,280,822 | Biotech, 2015 |
| HS2 | GCCCTGTAAGCAT | 37 | chr11: 5,280,668- | Hilton et al., Nat |
| Enhancer-4 | CCTGCTG | | 5,280,687 | Biotech, 2015 |
| SOCS1 +15 kb | GATTTCTAAGAAA | 38 | chr16: 11,240,852- | Mahata et al., |
| Enhancer -1 | CCAGTTG | | 11,240,871 | Nat Methods, |
| | | | | 2023 |
| SOCS1 +15 kb | TTCTGGAGCTGGA | 39 | chr16: 11,240,925- | Mahata et al., |
| Enhancer-2 | GCAAAGC | | 11,240,944 | Nat Methods, |
| | | | | 2023 |
| SOCS1 +15 kb | GATTTCTAAGAAA | 40 | chr16: 11,240,852- | Mahata et al., |
| Enhancer-3 | CCAGTTG | | 11,240,871 | Nat Methods, |
| | | | | 2023 |
| SOCS1 +50 kb | CTTGACCTGGAAG | 41 | chr16: 11,206,702- | Mahata et al., |
| Enhancer-1 | ACTCAGC | | 11,206,721 | Nat Methods, |
| | | | | 2023 |
| SOCS1 +50 kb | CTGAGAACGACC | 42 | chr16: 11,206,779- | Mahata et al., |
| Enhancer-2 | CCTAGGAG | | 11,206,798 | Nat Methods, |
| | | | | 2023 |
| NET1_Enhancer- | GAGGAATGTGCA | 43 | chr10: 5,493,900- | Zhang et al., Nat |
| 1 | AACGGCCC | | 5,493,919 | Comm., 2019 |
| NET1_Enhancer- | AGCCTACACCCTG | 44 | chr10: 5,493,772- | Zhang et al., Nat |
| 2 | GAGGTAC | | 5,493,791 | Comm., 2019 |
| GRASLND_ | CCACTGGGGATA | 45 | chr2: 6,918,864- | Huynh et al., |
| Promoter-1 | GTTCCCTG | | 6,918,883 | eLife, 2020 |
| GRASLND_ | TGACACCGTGAA | 46 | chr2: 6,919,268- | Mahata et al., |
| Promoter-2 | CTTTCCCA | | 6,919,287 | Nat Methods, |
| | | | | 2023 |
| GRASLND_ | TAAGTTGGTGTAG | 47 | chr2: 6,919,064- | Mahata et al., |
| Promoter-3 | ACGCCTG | | 6,919,083 | Nat Methods, |
| | | | | 2023 |
| GRASLND_ | TGAATTCTAGGCC | 48 | chr2: 6,919,475- | Mahata et al., |
| Promoter-4 | AAGACTT | | 6,919,494 | Nat Methods, |
| | | | | 2023 |
| TTN_ | CCTTGGTGAAGTC | 49 | chr2: 178,807,596- | Chavez et al., |
| Promoter-1 | TCCTTTG | | 178,807,615 | Nat Methods, |
| | | | | 2015 |
| rs72928038-1 | GGTGGGGCATCTT | 50 | chr6: 90,266,986- | |
| | TATAGCT | | 90,267,005 | |
| rs72928038-2 | ACATTACACACA | 51 | chr6: 90,267,019- | |
| | AATAGGGA | | 90,267,038 | |
| rs72928038-3 | GTTGAGGGTTTTT | 52 | chr6: 90,267,113- | |
| | TGGTGGG | | 90,267,132 | |
| AAVS1 | GTCCCCTCCACCC | 53 | chr19: 55,115,771- | Mali et al., |
| | CACAGTG | | 55,115,790 | Science 2013 |
| Non-targeting | GTATTACTGATAT | 54 | none | Doench et al., |
| gRNA | TGGTGGG | | | Nat. Biotech. |
| | | | | 2016 |
| Acidaminococcus sp. Cas12a crRNA | | | | |
| sequences used in this study. | | | | |
| | crRNA Spacer | | | |
| | Sequence | | Genomic Location | |
| | (dAsCas12a) | | (GRCh38/hg38 | |
| Target Gene | (5′-3′) | | Assembly) | Reference |
| ILIB_crRNA1 | CATGGTGATACAT | 55 | chr2: 112,836,988- | Campa et al., Nat |
| | TTGCAAA | | 112,837,007 | Methods, 2019 |
| Type V-F Cas12f (dCasMINI) | | | | |
| gRNA sequences used in this study. | | | | |
| | crRNA Spacer | | | |
| | Sequence | | Genomic Location | |
| | (dAsCas12a) | | (GRCh38/hg38 | |
| Target Gene | (5′-3′) | | Assembly) | Reference |
| HBG1 | CATTGAGATAGTG | 56 | chr11: 5,250,037- | Xu et al., Mol |
| | TGGGGAAGGG | | 5,250,059 | Cell, 2021 |

Transfection Strategies. RT-qPCR and Western blot experiments: Transient transfections were performed in 24-well plates using 375ng of respective dCas9 expression vector and 125 ng of single gRNA sectors or equimolar pooled gRNA expression vectors. Plasmids were mixed with Lipofectamine 3000 (Invitrogen, L3000015) as per the manufacturer's instruction Cells were harvested for analysis 72 hours post-transfection.

Immunoprecipitation experiments: HFK293T cells were transfected in 15 cm dishes with Lipofectamine 3000 and 37.5 μg of respective dCas9 expression vector and 12.5 μg of scrambled gRNA expression sector as per manufacturer instruction. Cells were harvested for analysis 48 hours post-transfection.

Single construct flow cytometry validation Transient transfections were performed in 24-well plates using 500ng rTetR-p300 variant expression vectors. Plasmids were mixed with Lipofectamine 3000 (Invitrogen, L3000015) as per the manufacturer's instruction. Cells were harvested for flow cytometry 48 hours post-transfection.

Prime Editing experiments: On Day 0, 1.5e5 HEK293T cells were plated in a 24-well plate. On Day 1, cells were transfected with 187.5ng of respective dCas9 expression vector, 187.5 ng of PEmax expression vector (Addgene #:) and 125 ng of pegRNA targeting the region of interest using Lipofectamine 3000. On Day 3, cells were passaged into a new 24-well plate. On Day 5, cells were harvested for analysis. A similar strategy was used for K562 cells using 2e5 cells nucleofected (Lonza 4D Nucleofector) with identical amounts of DNA and harvested on the same timeline as the HEK293T experiment.

Western blotting. Cells were lysed in RIPA buffer (Thermo Scientific, 89900) with 1× protease inhibitor cocktail (Thermo Scientific, 78442), lysates were cleared by centrifugation, and protein quantitation was performed using the BCA method (Pierce, 23225). 15-50 μg of lysate were separated using precast 7.5%, 10%, or 4-20% SDS-PAGE (Bio-Rad) and then transferred onto PVDF membranes using the Transblot-turbo system (Bio-Rad). Membranes were blocked using 5% BSA in 1×TBST and incubated overnight with primary antibody (anti-Cas9; 1:1000 dilution, Diagenode #C15200216. Anti-FLAG; 1:2000 dilution, Sigma-Aldrich #F1804, anti-β-Tubulin; 1:1000 dilution, Bio-Rad #12004166). Then membranes were washed with 1×TBST 3 times (5 mins each wash) and incubated with respective HRP-tagged secondary antibodies (1:2000 dilution) for 1 hr. Next membranes were washed with 1×TBST 3 times (5 mins each wash). Membranes were then incubated with ECL solution (BioRad #1705061) and imaged using a Chemidoc-MP system (BioRad). The β-tubulin antibody was tagged with Rhodamine (Bio-Rad #12004166) and was imaged using Rhodamine channel in Chemidoc-MP as per manufacturer's instruction.

Reverse-transcription quantitative PCR (RT-qPCR). RNA (including pre-miRNA) was isolated using the RNeasy Plus mini kit (Qiagen #74136). 500-2000ng of RNA (quantified using Nanodrop 3000C; Thermo Fisher) was used as a template for cDNA synthesis (Bio-Rad #1725038). cDNA was diluted 10× and 4.5 μL of diluted cDNA was used for each qPCR reaction in 10 μL reaction volume. Real-time quantitative PCR was performed using SYBR Green Master Mix (Bio-Rad #1725275) in the CFX96 Real-Time PCR system with a C1000 Thermal Cycler (Bio-Rad). Results are represented as fold change above control after normalization to GAPDH in all experiments using human cells. For murine cells, 18s rRNA was used for normalization. Undetectable samples were assigned a Ct value of 45 cycles.

Deep Mutational Scanning library Design. A deep mutational scanning library was designed including all single amino acid substitutions and deletions within the 1381-1420 residue positions of the p300 full-length protein. Amino acid sequences were reverse translated into DNA sequences using DNAChisel, a Python library for optimizing sequences with constraints and optimization objectives, as described previously (Tycko et al., 2020; Zulkower & Rosser, 2020). During the sequence optimization step, codons were optimized. Esp3l sites were removed, and the GC content was constrained to be between 30 and 70% for every 80-nucleotide window. In total, the library consisted of 841 variants of p300 and 100 random controls.

Library Cloning. Oligonucleotides were synthesized as pooled libraries (Twist Biosciences) and PCR amplified. 50 uL reactions were prepared with 5ng of template, 2.5 uL of each 10 mM primer, 10 uL of Q5 Reaction Buffer (NEB), 10 μL of Q5 High GC Enhancer (NEB), 1 uL of 10 nM DNTPs, and 0.5 uL of Q5 High Fidelity DNA Polymerase. PCR amplification parameters were as follows: 3 minutes at 98° C., then 29× cycles of 98′C for 10 sec, 61° C. for 30 sec, 72° C. for 30 sec, and a final step of 72° C. for 2 minutes. Resulting DNA was gel extracted using a QIAgen gel extraction kit. Libraries were then cloned into a vector for lentiviral recruitment (rTetR-p300Entry) with 4×10 uL Golden Gate reactions with each containing 75ng of predigested recruitment vector, 5ng of gel library, 0.13 uL of T4 DNA Ligase (NEB), 0.75 uL of Esp31 (NEB), and 1 uL of T4 DNA ligase buffer. Thermocycling parameters were as follows: 30× cycles at 37° C., and 16° C. for 5 minutes each followed by a 5-minute final digestion and heat inactivation for 20 minutes at 70° C. Reactions were pooled, purified using a DNA Clean and Concentrator-5 (Zymo), and eluted in 6 uL of molecular grade water. Two tubes of 50 uL Endura Electrocompetent Cells (Lucigen, Catalog #60242-2) were transformed with 2 uL per tube of column-purified libraries as per manufacturer instruction. After recovery, cells were plated on 3 large 15″ LB plates with ampicillin along with 3×10″ LB plates with ampicillin with serial dilutions of the transformed culture to confirm maintenance of 30× library coverage. After overnight growth, plates were scraped and extracted with a Qiagen Maxiprep Kit.

Lentivirus Production. One day before transfection, HEK293T cells were seeded at ~40% confluency in a 10-cm plate. The next day cells were transfected at ~80-90% confluency. For each transfection, 10 μg of plasmid containing the vector of interest, 10 μg of pMD2.G (Addgene, 12259), and 15 μg of psPAX2 (Addgene, 12260) were transfected using Lipofectamine 3000 (Invitrogen, L3000015) or calcium phosphate 14 hours post-transfection the media was changed. Supernatant was harvested 24 and 48 h post-transfection and filtered with a 0.45-μm PVDF filter (Millipore, SLGVM33RS), and then virus was concentrated at 100× using Lenti-X™ Concentrator (Takara, 631232), aliquoted and stored at −80° C. Lentiviral titers were measured by the Lenti-X™ qRT-PCR Titration Kit (Takara, 31232).

Measurement of Library Expression and Transcriptional Activity. 5M reporter HEK293T cells expressing 9×TetO-SV40 core were transduced with the lentiviral library containing the library of p300 variants on Day 0 by reverse transduction with 10 ug/mL polybrene. On Day 2, transduced cells were selected with 10ug/mL blasticidin. Cells were maintained in 3 15 cm² tissue culture plates. On Day 7, cells were treated with 1 ug/mL doxycycline for 2 days. On Day 9, cells were collected for cell sorting. Citrine and mCherry levels were measured on a Sony MA900 cell sorter and ~100 k cells were collected per bin corresponding to mChe^{lo}/Cit^{lo}, mChe^{lo}/Cit^{hi}, mChe^{hi}/Cit^{lo}, mChe^{hi}/Cit^{hi}.

Library preparation and sequencing. Genomic DNA was extracted with a DNEasy Blood and Tissue Kit (QIAgen) following manufacturer's protocol for each bin extracted from the sorting experiment (at 1e5 cells). DNA was eluted in EB and sequences were amplified by PCR. A two-step PCR process was used to append Illumina adapters as overhangs. 6× reactions were prepared with 1 ug of genomic DNA, 5 uL of each 10 uM primer, and 25 μL of NEBnext 2× Master Mix (NEB) was used for the first reaction. PCR amplification parameters were as follows: 3 minutes at 98° C., then 21× cycles of 98° C. for 10 sec, 68° C. for 30 sec, 72° C. for 30 sec, and a final step of 72° C. for 2 minutes. Resulting DNA was gel extracted using a QIAgen Gel Extraction Kit (QIAgen). A second PCR was performed with the Illumina indexed primers with the following PCR amplification parameters: 3 minutes at 9° C., then 11× cycles of 98° C. for 10 sec, 68° C. for 30 sec, 72° C. for 30 sec, and a final step of 72° C. for 2 minutes. Resulting DNA was again gel extracted using a QIAgen Gel Extraction Kit (QIAgen). Libraries were then quantified with a Qubit HS dsDNA High Sensitivity Kit (Thermo Fisher), pooled with 15% PhiX control (Illumina), and sequenced on an Illumina HiSeq 3000.

Immunoprecipitation and FLAG Pulldown. Cells were harvested with a cell scraper and spun at 300×g for 5 min. The packed cell volume was measured and 2.5 volumes of NETN buffer was added to the cell pellet. (NETN: 50 mM Tris pH 7.3, 170 mM NaCl, 1 mM EDTA, 0.5% NP-40). Lysate was sonicated (30 sec bust. 59 sec off, repeat 6 times) using a Diagenode Bioruptor Pico (B01060010) and then centrifuged at 20,000×g, for 20 min at 4° C. Supernatant was harvested and then transferred to a fresh tube.

5 μg of FLAG antibody (F1804-200UG) was added to the above supernatant and incubated for I hour at 4′C rocking. Supernatant was spun again at 20,000×g for 20 min and the supernatant was collected in a fresh tube. 20 μl of protein A bead slurry was added to the above supernatant and incubated for 1 hour at 4° C. with inverted rotation. Supernatant and beads were spun at 1000×g for 1 min. Flow-through was saved for downstream QC testing. Beads were washed with 1 ml NETN buffer and spun at 1000×g for Imin and supernatant was discarded A flat-ended tip was used to remove residual buffer solution from the beads completely. Add 20 μl of 2'SDS loading dye to the beads. Heat samples for 8-10 min at 90 C.

Mass Spectrometry. The immunoprecipitated samples were resolved on NuPAGE 10% Bis-Tris Gel (Life Technologies), each lane was excised into 6 equal pieces and combined into two peptide pools after in-gel digestion using trypsin enzyme. The peptides were dried in a speed vac and dissolved in 5% methanol containing 0.1% formic acid buffer. The LC-MS/MS analysis was carried out using the nano-LC 1000 system coupled to Orbitrap Fusion mass spectrometer (Thermo Scientific). Peptides were eluted on an analytical column (20 cm×75 μm I.D.) filled with Reprosil-Pur Basic C18 (1.9 μm, Dr. Maisch GmbH, Germany) using 110 minutes discontinuous gradient of 90% acetonitrile buffer (B) in 0.1% formic acid at 200 nl/min (2-30% B: 86 min, 30-60% B: 6 min, 60-90% B: 8 min, 90-50% B: 10 min). The full MS scan was performed in Orbitrap analyzer in the range of 300-1400m/z at 120,000 resolution followed by an IonTrap HCD-MS2 fragmentation for a cycle time of 3 seconds with precursor isolation window of 3m/z, collision energy 30%, AGC of 50000, maximum injection time of 30 ms.

The MS raw data were searched using Proteome Discoverer 2.0 software (Thermo Scientific) with Mascot algorithm against human NCBI RefSeq database updated 2020_0324. The precursor ion tolerance and product ion tolerance were set to 20 ppm and 0.5 Da, respectively. Maximum cleavage of 2 with Trypsin enzyme, dynamic modification of oxidation (M), protein N-term acetylation, deamidation (N/Q) and destreak (C) was allowed. The peptides identified from the mascot result file were validated with a 5% false discovery rate (FDR). The gene product inference and quantification were done with label-free iBAQ approach using the ‘gpGrouper’ algorithm. For statistical assessment, missing value imputation was employed through sampling a normal distribution N (μ-1.8 σ, 0.8σ), where μ, σ are the mean and standard deviation of the quantified values. For differential analysis, the moderated t-test and log 2 fold changes as were used implemented in the R package limma (Tycko et al. 2020) and multiple-hypothesis testing correction was performed with the Benjamini-Hochberg procedure. To filter out background contaminants, proteins that were recovered in more than 50% of IP analyses in HEK293T cells were removed from further analysis according to the contaminant repository for affinity purification (CRAPome (Mellacheruvu et al., 2013)). To quantify shared hits in the STRING database, all interactors of p300 with a combined score >0.15 were extracted and compared against hits found in this study's IP-MS dataset.

RNA-Sequencing. RNA sequencing (RNA-seq) was performed in duplicate for each experimental condition. RNA was isolated from transfected cells using the RNeasy Plus mini kit (Qiagen, 74136) RNA-seq libraries were constructed using the TruSeq Stranded Total RNA Gold (Illumina, RS-122-2303). The qualities of RNA-seq libraries were verified using the Tape Station D1000 assay (Tape Station 2200, Agilent Technologies), and the quantities of RNA-seq libraries were checked again using real-time PCR (QuantStudio 6 Flex Real time PCR System, Applied Biosystem). Libraries were normalized and pooled and then 75 bp paired-end reads were sequenced on the HiSeq3000 platform (Illumina). Sequencing reads were aligned to the human genome (GRCh38.p13) using the STAR (Spliced Transcripts Alignment to a Reference, version 2.7.9a) against the Gencode Release 36 primary assembly annotation (Liao et al., 2014). Sequence alignment was conducted on an Amazon Web Services Elastic Compute Cloud Instance (EC2) with 64 GB of memory and 256 GB of storage. Read quantification and differential expression analysis were conducted on R (version 4.2.2). Read quantification was carried out with featureCounts (Love et al., 2014). Differential expression analysis was performed using DESeq2 (Wu et al., 2023). Volcano plots, PCA plots, and heat maps were created on DESeq2 using the analyzed data.

CUT&RUN. CUT&RUN was performed using the Epicypher CUTANA ChIC/CUT&RUN Kit (Epicypher, #14-1048). Briefly, 500 k HEK293T cells were detached and harvested using 0.5 mM EDTA (Fisher, #BP2482-500), washed once with 1×PBS and then resuspended in 300ul of wash buffer. Next each of 3 100ul aliquots (~⅓ of each 24 well) of cells were processed for H3K4me3 antibody (Epicypher, #13-0041), H3K27ac antibody (Epicypher, #13-0045), H3K27me3 antibody (Epicypher, #13-0055), H2BK20ac antibody (Abcam, ab177430), or input DNA, respectively. Cells were first immobilized on concanavalin A beads, and then incubated with respective antibody (0.5ug/sample) overnight at 4° C. in antibody dilution buffer (cell permeabilization buffer+EDTA). On the following day, cells were washed twice with cell permeabilization buffer. After washing the beads, pAG-MNase was added to the immobilized cells and then incubated for 2 hours at 4° C. to digest and release DNA. Libraries were prepared using the CUT&RUN Library Prep Kit (Epicypher, #14-1001) and DNA was quantified using the Qubit HS dsDNA High Sensitivity Kit (Thermo Fisher), and sequenced at a depth of 15M paired end reads per sample on a NextSeq 2000 (High Output 2×100). Samples were normalized using an E. coli spike-in DNA (Epicypher, #18-1401).

For CUT&RUN-qPCR assays, purified DNA from either H3K4me3, H3K27ac, H3K27me3, or H2BK20ac antibody incubated samples were then assayed by qPCR. Relative enrichment of H3K4me3 and H3K27ac is expressed as fold change above control cells transfected with dCas9 plasmid and after normalization to purified input DNA.

scCRISPRa transduction. Monoclonal K562 cell lines expressing the construct of interest were generated by transducing cells (8ug/mL) at a range of % v/v and performing flow cytometry 2 days later. Wells receiving dilutions with ~40% mCherry+ were selected and plated for monoclonal lines by limiting dilution. Monoclonal lines were transduced (10 ug/mL) with varying titers to assess viral copy number. Library transduction with a 1.5% v/v was selected for the scCRISPRa experiment. For this experiment, 500 k cells were transduced with virus containing the sgRNA library. Cells were spun out of polybrene-containing media and replaced with standard K562 culture media. At 2 days post-transduction, I ug/mL puromycin was added to the culture. 9 days after transduction, cells were collected for scRNA-seq.

10× Genomics scRNA-sequencing with gRNA Capture. Cells were harvested and prepared as per the 10× Genomics Single Cell Protocols Cell Preparation Guide. ~10,000 cells were captured per lane using a 10× Chromium device. One lane was used per construct (dCas9-p300 WT and dCas9-p300 I1417N). Cells were captured using a 10× Chromium chip using the Chromium Next GEM Single Cell 3′ Reagents Kit v.3 with Feature Barcoding Technology for CRISPR screening.

Sequencing of scRNA-seq libraries. Final libraries were sequenced using a NovaSeq 6000 for each p300 screen. Gene expression and CRISPR Guide Capture transcript libraries were pooled at a 4:1 ratio for sequencing.

Transcriptome and sgRNA data processing and QC. Cell Ranger Count v7.1.0 was used to perform count matrix generation using default parameters. Human (GRCh38) 2020-A was used for the transcriptome reference. Cells with less than 10% mitochondrial reads and between 750 and 7000 genes detected were kept for analysis. Resulting processed matrices were used for all downstream analyses.

Gene Expression Quantification. Each of the sequencing datasets was processed using Seurat (Butler et al., 2018). Cells with greater than 10% mitochondrial transcripts, or less than 4000 total RNA transcripts were removed. For each gRNA in the library, expression levels were gathered for all genes within 1 Mb of the gRNA target site. For each gRNA-target gene pairing, the FindMarkers( ) function was used to calculate the log 2( ) fold changes in expression using the following parameters: ident.1=gRNA_Cells, ident.2=Control_Cells, min.pct=0, min.cells.feature=0, min.cells.group=0, features=target_gene, logfc.threshold=0. Violin plots were created using Seurat on the top few hits by adjusted p-value for each dataset analyzed. To make the violin plots, cells were divided into those with the gRNA present and those without, and the resulting expression levels for the target gene for each set of cells can be visualized.

High-MOI gRNA Assignment and Differential Expression Testing using SCEPTRE. SCEPTRE was implemented as described previously^{70,71}. The processed count matrices were used along with single-cell metadata as covariates to fit the SCEPTRE model. gRNA-response pairs were tested for differential expression for all genes within 1 MB upstream and 1 Mb downstream of the gRNA of interest. SCEPTRE p-values were adjusted with the Benjamini-Hochberg procedure and target genes were identified if they were significancy below a threshold value of 10% FDR.

Quantification and Statistical Analysis. Statistical information for all experiments is found in the figure legends. Illustrations were done using BioRender and Adobe Illustrator.

### Example 4—Engineered p300 for Liver Disease

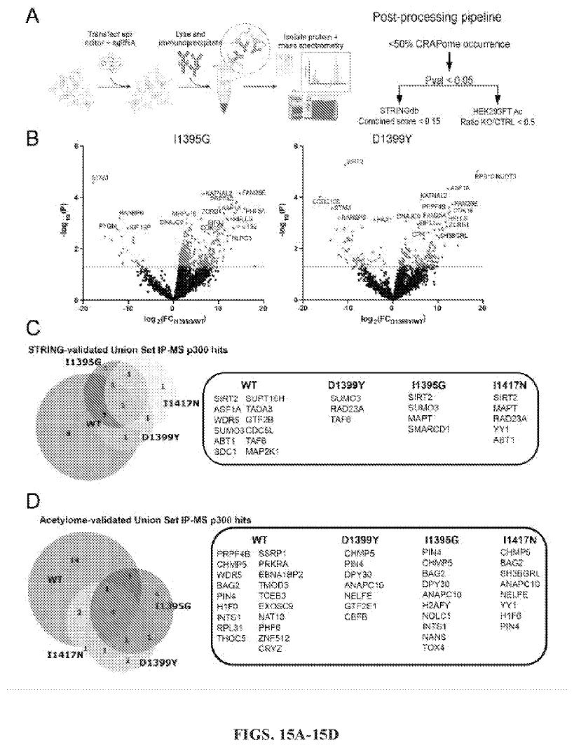

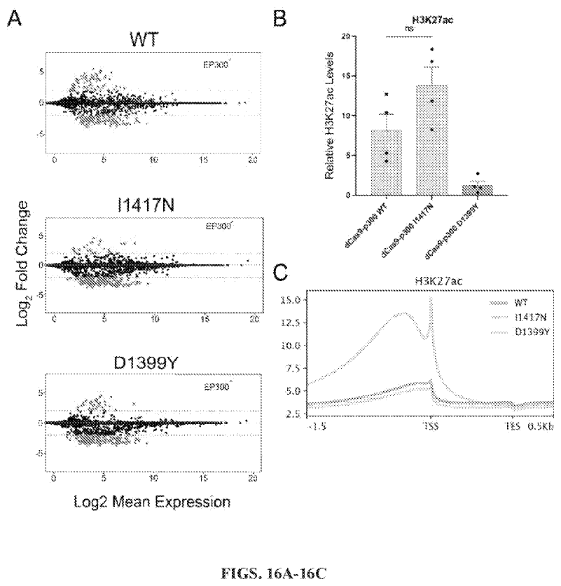

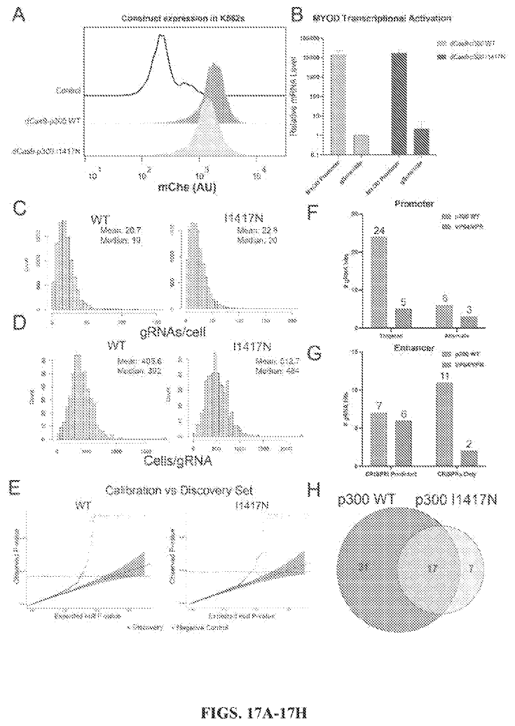

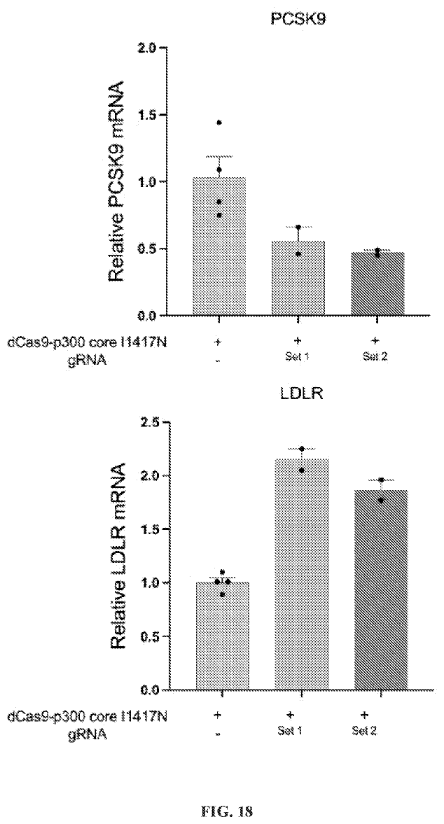

In this study, dCas9-p300 I1417N was targeted to a gene linked to lipid metabolism and familial hypercholesterolemia in liver cells as a therapeutic application of the present epigenome editing approach for chronic liver diseases. dCas9-p300 I1417N was delivered to Huh7 cells by lentivirus and a stable polyclonal cell line was generated expressing this epigenome editor. Guide RNAs were then delivered to target the promoter upstream of LDLR. This resulted in a 2-fold activation of gene expression and a concomitant decrease of PCSK9 by 50%, indicative of a previously known negative regulatory network between the two genes that controls LDL uptake (FIG. 18). This validates the approach of activating gene expression using the engineered p300 construct.

Next, a fusion protein of dCasMINI to p300 I1417N was generated Because the genetic footprint of dCas9 is large (~4.1 kb), the smaller size of dCasMINI (~1.7 kb) was used to enable AAV delivery which has a packaging limit of ~4.6 kb alongside the p300 I1417N construct (1.8 kb). The function of a dCasMINI-p300 I1417N construct was confirmed in HEK293T cells when targeted to the HBG1 promoter.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
International Patent Publication No. WO9324640
International Patent Publication No. WO94/016737
U.S. Pat. No. 5,593,972
U.S. Pat. No. 5,962,428
U.S. Patent Publication No. US20040175727
Ali et al., Chem. Rev. 118, 1216-1252 (2018).
Barry et al., Genome Biology 22, 344 (2021).
Black et al., Molecular Cell 32, 449-455 (2008).
Bordoli et al., Nucleic Acids Research 29, 4462-4471 (2001).
Bulusu et al., Cell Reports 18, 647-658 (2017).
Campa et al., Nat Methods 16, 887-893 (2019).
Chardon et al., 2023.03.28.534017 (2023).
Dai et al., EMBO reports 22, e52023 (2021).
Dancy & Cole, Chem. Rev. 115, 2419-2452 (2015).
Delvecchio et al., Nat Struct Mol Biol 20, 1040-1046 (2013).
Dominguez et al., The CRISPR Journal 5, 264-275 (2022).
Dutto et al., Cell Mol Life Sci 75, 1325-1338 (2018).
Eldridge et al., Cell Rep 18, 1285-1297 (2017).
Esvelt et al., Nature Methods, 10(11), 1116-21 (2013).
Fellows et al., Nat Commun 9, 105 (2018).
Filippakopoulos et al., Cell 149, 214-231 (2012).
Flynn et al., Structure 23, 1801-1814 (2015).
Fulco et al., Science 354, 769-773 (2016).
Gasperini et al., Cell 176, 377-390.e19 (2019).
Gasperini et al., Nat Rev Genet 21, 292-310 (2020).
Gemberling et al., Nat Methods 18, 965-974 (2021)
Gillespie et al., Molecular Cell 78, 960-974.e11 (2020).
Goell & Hilton, Trends in Biotechnology 39, 678-691 (2021).
Goudarzi et al., Molecular Cell 62, 169-180 (2016).
Haberle et al., Nature 570, 122-126 (2019).
Hilton et al., Nat Biotechnol 33, 510-517 (2015).
Hsu et al., Nature Biotechnology, 31, 827-832 (2013).
Hofacker et al., International Journal of Molecular Sciences 21, 502 (2020).
Huang et al., Nature 597, 132-137 (2021).
Hnisz et al., Cell 155, 934-947 (2013).
Jain et al., 2022.02.28.482307 (2023).
Jones et al., Nat Commun 11, 5690 (2020).
Kaczmarska et al., Nat Chem Biol 13, 21-29 (2017).
Klann et al., Nat Biotechnol 35, 561-568 (2017).
Li et al., Molecular Cell 62, 181-193 (2016).
Li et al., Nat Commun 11, 485 (2020).
Li et al., 2023.04.12.536587 (2023).
Liao et al., Bioinformatics 30, 923-930 (2014).
Lin et al., Toxicological Sciences 96, 83-91 (2007).
Liu et al., Cell Discov 3, 1-17 (2017).
Liu et al., Molecular Cell 83, 2206-2221.e11 (2023).
Love et al., Genome Biology 15, 550 (2014).
Mahata et al., Nat Methods 1-13 (2023).
Maksimoska et al., Biochemistry 53, 3415-3422 (2014).
Martin et al., Nat Commun 12, 210 (2021).
Matharu et al., Science 363, eaau0629 (2019).
Maunder et al., Nat Commun 8, 14834 (2017).
Mellacheruvu et al., Nat Methods 10, 730-736 (2013).
Millán-Zambrano et al., Nat Rev Genet 23, 563-580 (2022).
MofTat et al., Cell 124, 1283-1298 (2(06).
Morris et al., Science 380, eadh7699 (2023).
Nakamura et al., Nat Cell Biol 23, 11-22 (2021).
Narita et al., Nat Genet 55, 679-692 (2023).
Nitsch et al., EMBO reports 22, e52774 (2021).
Nuñez et al., Cell 184, 2503-2519.e17 (2021).
O'Geen et al., Nucleic Acids Research 45, 9901-9916 (2017).
Olzscha et al., Cell Chemical Biolog 24, 9-23 (2017).
Ortega et al., Nature 562, 538-544 (2018).
Pflueger et al., Genome Res. 28, 1193-1206 (2018).
Policarpi et al., 2022.09.04.506519 (2022).
Sabari et al., Mol Cell 58, 203-215 (2015).
Sabari et al., Science 361, eaar3958 (2018).
Sambrook et al., Molecular Cloning and Laboratory Manual, Second Ed., Cold Spring Harbor, 1989.
Schulze et al., Biochem. Cell Biol. 87, 65-75 (2009).
Sen et al., Mol Cell 73, 684-698.e8 (2019).
Sheikh & Akhtar, Nat Rev Genet 20, 7-23 (2019).
Shvedunova & Akhtar, Nat Rev Mol Cell Biol 23, 329-349 (2022).
Slabicki et al., Nature 585, 293-297 (2020).
Soaita et al., Journal of Biological Chemistry 299, (2023).
Tan et al., Cell 146, 1016-1028 (2011).
Tate et al., Nucleic Acids Research 47, D941-D947 (2019).
Trefely et al., Molecular Metabolism 38, 100941 (2020).
Tycko et al., Cell 183, 2020-2035.e16 (2020).
Tycko et al., 2023.05.12.540558, (2023).
Wang et al., Nucleic Acids Research 50, 7842-7855 (2022)
Weinert et al., Cell 174, 231-244.e12 (2018).
Wu et al., Cell Death Dis 10, 1-15 (2019).
Wu et al., Molecular Cell 83, 1125-1139.e8 (2023).
Xu et al., Molecular Cell 81, 4333-4345.e4 (2021)
Yao et al., 2022.12.21.520137 (2022).
Zhang et al., Genome Biology 21, 45 (2020).
Zhao et al., Sci Rep 11, 15912 (2021).
Zimmermann et al., Eur J Hum Genet 15, 837-842 (2007).
Zulkower & Rosser, Bioinformatics 36, 4508-4509 (2020).

What is claimed:

1. A polypeptide comprising an engineered human lysine acyltransferase or effector domain thereof comprising at least one amino acid mutation in the acyl-CoA binding site.

2. The polypeptide of claim 1, wherein the engineered human lysine acyltransferase is an engineered human lysine acetyltransferase (KAT), an engineered lysine crotonyltransferase, p300 or CBP.

3. The polypeptide of any of claims 1-2, wherein the at least one amino acid mutation is (a) between amino acids 1380-1420 and/or (b) an I1417N, I1395G, P1388C, Y1397Q, Y1397A, Q1390R, K1407Y, L1409K, and/or C1385Q mutation.

4. The polypeptide of any one of claims 2-3, wherein the p300 comprises an I1417N mutation and/or the p300 comprising a I1417N mutation has decreased cellular toxicity as compared to endogenous p300.

5. The polypeptide of any one of claims 1-5, wherein the lysine acyltransferase, lysine acetyltransferase (KAT), or lysine crotonyltransferase is fused to (a) Tetracycline repressor (TetR) protein, (b) a (Clustered Regularly Interspaced Short Palindromic Repeats associated) Cas protein, (c) a Zinc finger or TALE protein, and/or (d) a reporter.

6. The polypeptide of claim 5, wherein the Cas protein is dCas9, ddCas12a, dCasMINI, dCas12f, dCas12i, or dCas12j, and/or the reporter is a fluorescent protein.

7. A fusion protein comprising the engineered polypeptide of any one of claims 1-6 and a Cas polypeptide, Zinc finger polypeptide, or TALE polypeptide.

8. The fusion protein of claim 7, wherein
(a) the Cas polypeptide is dCas9, ddCas12a, dCasMINI, dCas12f, dCas12i, or dCas12j;
(b) the fusion protein further comprises a linker between the engineered KAT and the Cas polypeptide, Zinc finger polypeptide, or TALE polypeptide; and/or
(c) the fusion protein activates transcription of a target gene by activating distal regulatory elements.

9. An isolated polynucleotide encoding the fusion protein of claim 7 or 8.

10. A vector comprising the isolated polynucleotide of claim 9.

11. The vector of claim 10, wherein the vector is a viral vector or mRNA, wherein the viral vector is a lentiviral vector, an adenoviral vector, a retroviral vector, a vaccinia viral vector, an adeno-associated viral (AAV) vector, a herpes viral vector, or a polyoma viral vector and/or the vector is a lentiviral vector with increased packaging and transduction efficiency as compared to a lentiviral vector encoding endogenous p300.

12. A DNA targeting system comprising the fusion protein of 8 or 9 and at least one guide RNA (gRNA).

13. The DNA targeting system of claim 12, wherein the at least one gRNA targets a target region, wherein the target region comprises a target enhancer, target regulatory element, a cis-regulatory region of a target gene, or a trans-regulatory region of a target gene and/or wherein the DNA targeting system comprises (a) between one and ten different gRNAs or (b) one gRNA.

14. The DNA targeting system of claim 13, wherein the target region is:
(a) a distal or proximal cis-regulatory region of the target gene;
(b) an enhancer region or a promoter region of the target gene;
(c) an endogenous gene or a transgene;
(d) located on the same chromosome as the target gene;
(e) located about 1 base pair to about 1,000,000 base pairs, about 1 base pair to about 100,000 base pairs, or about 1000 base pairs to about 50,000 base pairs upstream of a transcription start site of the target gene; or
(f) located on a different chromosome as the target gene.

15. The DNA targeting system of any one of claims 12-14, wherein the target region is:
(a) at least one of HS2 enhancer of the human β-globin locus, distal regulatory region (DRR) of the MYOD gene, core enhancer (CE) of the MYOD gene, proximal (PE) enhancer region of the OCT4 gene, or distal (DE) enhancer region of the OCT4 gene;
(b) low-density lipoprotein receptor (LDLR) gene, PCSK9, ATP7A, ATP7B, or a gene in Tables 1-3;
(c) the promoter of ANO5, IRS1, SCN2A, or EIF1AX; and/or
(d) the enhancer of PCTP, LINC01993, LGALS3BP, ZNF180, THUMPD2, LINC01033, SNX18, HIST1H4H, HIST1H2BO, or SLC35B2.

16. A method for activating gene expression of a target gene in a cell comprising contacting the cell with the polypeptide of any one of claims 1-6, the fusion protein of claim 7 or 8, a vector of claim 10 or 11, or a DNA targeting system of any one of claims 12-15.

17. The method of claim 16, wherein the cell is contacted with (a) a polypeptide of any one of claims 1-6 and a DNA-binding domain for the target gene, (b) a fusion protein of claim 7 or 8, (c) a vector of claim 10 or 11, or (d) a DNA targeting system of any one of claims 12-15 and at least one gRNA.

18. A method for cell therapy comprising activating a target gene by introducing the engineered lysine acyltransferase, lysine acetyltransferase (KAT), or lysine crotonyltransferase of any one of claims 1-6 and a DNA-binding domain for said target gene.

19. The method of claim 18, wherein the method is ex vivo, is in vivo, or reduces T cell exhaustion.

20. The method of claim 18, wherein the disease comprises haploinsufficiency and/or is nonalcoholic fatty liver disease (NASH), sickle cell disease, alpha thalassemia, beta thalassemia, chronic liver disease, Wilson's disease or a central nervous system (CNS) disease.
